SEQUENCE LISTING (35 sequences)

SEQ ID NO: 1 - DNA, 1680 bp, Artificial Sequence
  chemically synthesized
atggggcggccgcttctagagaattcgcggccgcttctagagctgcatataaagttactttggtaacaccaaccggtaatgtcgaatttcaatgtcctgatgacgtgtacattttagacgccgctgaggaagagggaatagatctaccatattcttgcagagcaggctcatgttccagttgcgccggtaagcttaaaactggaagcttgaaccaggatgaccaatctttcttagatgatgaccagatcgatgaaggctgggttctaacatgtgctgcataccctgtatcagacgtcaccattgaaactcataaggaggaagaacttacagccactagagctgcaccagccgcagaagctcctttgtctcatgttcaacaggccttagccgagcttgcaaaaccaaaggatgaccctactagaaaacacgtatgtgtccaagtggccccagctgttagggtagcaattgctgaaacacttggtttggcccctggagcaaccactccaaagcagttagctgagggcctaagaaggcttggttttgatgaagtgttcgacacattgtttggagccgatttaaccataatggaagagggctcagaattgttacatagactaactgaacaccttgaggcacatcctcactccgacgaaccattgcctatgttcacaagttgctgtccaggttggatcgctatgttagaaaaaagctatcctgatctaattccatacgtgagctcatgcaagtcccctcaaatgatgttggccgcaatggttaaaagttatttagctgagaagaaaggtatagccccaaaggatatggtaatggtcagcatcatgccatgtaccagaaaacaatctgaagcagacagggattggttttgcgttgacgctgatcctactcttagacagttggatcatgtgattacaaccgttgagttaggaaatatattcaaggaaagaggcatcaacctagccgaacttccagagggtgaatgggacaatcctatgggagtaggttcaggcgcaggtgtcttgtttggaactacaggcggcgtgatggaagctgctttaaggactgcctacgagctattcaccggtacaccattgcctagattatcccttagtgaagttaggggaatggatggtattaaagaaactaacattaccatggtaccagcacctggctctaagtttgaggaattgttaaaacatagagctgccgcaagagctgaagccgcagctcacggaacaccaggtcctctagcatgggacggcggtgctggattcactagcgaggatggtaggggcggcataacattgagagtcgccgttgcaaatggattaggtaacgctaaaaagcttatcaccaaaatgcaagccggcgaagcaaagtatgattttgtggagattatggcttgtccagccggatgtgttggtggaggcggacaacctagatcaactgacaaagcaataacacagaagaggcaagctgccctatacaatttggatgaaaaatccactttaagaagaagtcatgaaaacccatctatcagggagctttatgacacctacttgggtgaacctttaggtcacaaggcacatgaactattgcacacacattatgtagctggcgggtcgaggaaaaagatgaaaagaaaactagtagcggccgctgcag

SEQ ID NO: 2 - protein, 553 aa, Artificial Sequence
  chemically synthesized
EFAAASRAAYKVTLVTPTGNVEFQCPDDVYILDAAEEEGIDLPYSCRAGSCSSCAGKLKTGSLNQDDQSFLDDDQIDEGWVLTCAAYPVSDVTIETHKEEELTATRAAPAAEAPLSHVQQALAELAKPKDDPTRKHVCVQVAPAVRVAIAETLGLAPGATTPKQLAEGLRRLGFDEVFDTLFGADLTIMEEGSELLHRLTEHLEAHPHSDEPLPMFTSCCPGWIAMLEKSYPDLIPYVSSCKSPQMMLAAMVKSYLAEKKGIAPKDMVMVSIMPCTRKQSEADRDWFCVDADPTLRQLDHVITTVELGNIFKERGINLAELPEGEWDNPMGVGSGAGVLFGTTGGVMEAALRTAYELFTGTPLPRLSLSEVRGMDGIKETNITMVPAPGSKFEELLKHRAAARAEAAAHGTPGPLAWDGGAGFTSEDGRGGITLRVAVANGLGNAKKLITKMQAGEAKYDFVEIMACPAGCVGGGGQPRSTDKAITQKRQAALYNLDEKSTLRRSHENPSIRELYDTYLGEPLGHKAHELLHTHYVAGGVEEKDEKKTSSGRC

SEQ ID NO: 3 - protein, 689 aa, Artificial Sequence
  chemically synthesized
MGAAASRAAYKVTLVTPTGNVEFQCPDDVYILDAAEEEGIDLPYSCRAGSCSSCAGKLKTGSLNQDDQSFLDDDQIDEGWVLTCAAYPVSDVTIETHKEEELTATRKTIILNGNEVHTDKDITILELARENNVDIPTLCFLKDCGNFGKCGVCMVEVEGKGFRAACVAKVEDGMVINTESDEVKERIKKRVSMLLDKHEFKCGQCSRRENCEFLKLVIKTKAKASKPFLPEDKDALVDNRSKAIVIDRSKCVLCGRCVAACKQHTSTCSIQFIKKDGQRAVGTVDDVCLDDSTCLLCGQCVIACPVAALKEKSHIEKVQEALNDPKKHVIVAMAPSVRTAMGELFKMGYGKDVTGKLYTALRMLGFDKVFDINFGADMTIMEEATELLGRVKNNGPFPMFTSCCPAWVRLAQNYHPELLDNLSSAKSPQQIFGTASKTYYPSISGIAPEDVYTVTIMPCNDKKYEADIPFMETNSLRDIDASLTTRELAKMIKDAKIKFADLEDGEVDPAMGTYSGAGAIFGATGGVMEAAIRSAKDFAENKELENVDYTEVRGFKGIKEAEVEIAGNKLNVAVINGASNFFEFMKSGKMNEKQYHFIEVMACPGGCINGGGQPHVNALDRENVDYRKLRASVLYNQDKNVLSKRKSHDNPAIIKMYDSYFGKPGEGLAHKLLHVKYTKDKNVSKHETS

SEQ ID NO: 4 - DNA, 2085 bp, Artificial Sequence
  chemically synthesized
atgggcgcggccgcttctagagcggccgcttctagagctgcatataaagttactttggtaacaccaaccggtaatgtcgaatttcaatgtcctgatgacgtgtacattttagacgccgctgaggaagagggaatagatctaccatattcttgcagagcaggctcatgttccagttgcgccggtaagcttaaaactggaagcttgaaccaggatgaccaatctttcttagatgatgaccagatcgatgaaggctgggttctaacatgtgctgcataccctgtatcagacgtcaccattgaaactcataaggaggaagaacttacagccactagaaaaacaataatcttaaatggcaatgaagtgcatacagataaagatattactatccttgagctagcaagagaaaataatgtagatatcccaacactctgctttttaaaggattgtggcaattttggaaaatgcggagtctgtatggtagaggtagaaggcaagggctttagagctgcttgtgttgccaaagttgaagatggaatggtaataaacacagaatccgatgaagtaaaagaacgaatcaaaaaaagagtttcaatgcttcttgataagcatgaatttaaatgtggacaatgttctagaagagaaaattgtgaattccttaaacttgtaataaagacaaaagcaaaagcttcaaaaccatttttaccagaagataaggatgctctagttgataatagaagtaaggctattgtaattgacagatcaaaatgtgtactatgcggtagatgcgtagctgcatgtaaacagcacacaagcacttgctcaattcaatttattaaaaaagatggacaaagggctgttggaactgttgatgatgtttgtcttgatgactcaacatgcttattatgcggtcagtgtgtaatcgcttgtcctgttgctgctttaaaagaaaaatcccatatagaaaaagttcaagaagctcttaatgaccctaaaaaacatgtcattgttgcaatggctccatcagtaagaactgctatgggcgaattattcaaaatgggatatggaaaagatgtaacaggaaaactatatactgcacttagaatgttaggctttgataaagtatttgatataaactttggtgcagatatgactataatggaagaagctactgaacttttaggcagagttaaaaataatggcccattccctatgtttacatcttgctgtcctgcatgggtaagattagctcaaaattatcatcctgaattattagataatctttcatcagcaaaatcaccacaacaaatatttggtactgcatcaaaaacttactatccttcaatttcaggaatagctccagaagatgtttatacagttactatcatgccttgtaatgataaaaaatatgaagcagatattcctttcatggaaactaacagcttaagagatattgatgcatccttaactacaagagagcttgcaaaaatgattaaagatgcaaaaattaaatttgcagatcttgaagatggtgaagttgatcctgctatgggtacttacagtggtgctggagctatctttggtgcaaccggtggcgttatggaagctgcaataagatcagctaaagactttgctgaaaataaagaacttgaaaatgttgattacactgaagtaagaggctttaaaggcataaaagaagcggaagttgaaattgctggaaataaactaaacgttgctgttataaatggtgcttctaacttcttcgagtttatgaaatctggaaaaatgaacgaaaaacaatatcactttatagaagtaatggcttgccctggtggatgtataaatggtggaggtcaacctcacgtaaatgctcttgatagagaaaatgttgattacagaaaactaagagcatcagtattatacaaccaagataaaaatgttctttcaaagagaaagtcacatgataatccagctattattaaaatgtatgatagctactttggaaaaccaggtgaaggacttgctcacaaattactacacgtaaaatacacaaaagataaaaatgtttcaaaacatgaaactagttaa

SEQ ID NO: 5 - protein, 689 aa, Artificial Sequence
  chemically synthesized
MGAAASRKTIILNGNEVHTDKDITILELARENNVDIPTLCFLKDCGNFGKCGVCMVEVEGKGFRAACVAKVEDGMVINTESDEVKERIKKRVSMLLDKHEFKCGQCSRRENCEFLKLVIKTKAKASKPFLPEDKDALVDNRSKAIVIDRSKCVLCGRCVAACKQHTSTCSIQFIKKDGQRAVGTVDDVCLDDSTCLLCGQCVIACPVAALKEKSHIEKVQEALNDPKKHVIVAMAPSVRTAMGELFKMGYGKDVTGKLYTALRMLGFDKVFDINFGADMTIMEEATELLGRVKNNGPFPMFTSCCPAWVRLAQNYHPELLDNLSSAKSPQQIFGTASKTYYPSISGIAPEDVYTVTIMPCNDKKYEADIPFMETNSLRDIDASLTTRELAKMIKDAKIKFADLEDGEVDPAMGTYSGAGAIFGATGGVMEAAIRSAKDFAENKELENVDYTEVRGFKGIKEAEVEIAGNKLNVAVINGASNFFEFMKSGKMNEKQYHFIEVMACPGGCINGGGQPHVNALDRENVDYRKLRASVLYNQDKNVLSKRKSHDNPAIIKMYDSYFGKPGEGLAHKLLHVKYTKDKNVSKHETRAAYKVTLVTPTGNVEFQCPDDVYILDAAEEEGIDLPYSCRAGSCSSCAGKLKTGSLNQDDQSFLDDDQIDEGWVLTCAAYPVSDVTIETHKEEELTATS

SEQ ID NO: 6 - DNA, 2085 bp, Artificial Sequence
  chemically synthesized
atgggcgcggccgcttctagaaaaacaataatcttaaatggcaatgaagtgcatacagataaagatattactatccttgagctagcaagagaaaataatgtagatatcccaacactctgctttttaaaggattgtggcaattttggaaaatgcggagtctgtatggtagaggtagaaggcaagggctttagagctgcttgtgttgccaaagttgaagatggaatggtaataaacacagaatccgatgaagtaaaagaacgaatcaaaaaaagagtttcaatgcttcttgataagcatgaatttaaatgtggacaatgttctagaagagaaaattgtgaattccttaaacttgtaataaagacaaaagcaaaagcttcaaaaccatttttaccagaagataaggatgctctagttgataatagaagtaaggctattgtaattgacagatcaaaatgtgtactatgcggtagatgcgtagctgcatgtaaacagcacacaagcacttgctcaattcaatttattaaaaaagatggacaaagggctgttggaactgttgatgatgtttgtcttgatgactcaacatgcttattatgcggtcagtgtgtaatcgcttgtcctgttgctgctttaaaagaaaaatcccatatagaaaaagttcaagaagctcttaatgaccctaaaaaacatgtcattgttgcaatggctccatcagtaagaactgctatgggcgaattattcaaaatgggatatggaaaagatgtaacaggaaaactatatactgcacttagaatgttaggctttgataaagtatttgatataaactttggtgcagatatgactataatggaagaagctactgaacttttaggcagagttaaaaataatggcccattccctatgtttacatcttgctgtcctgcatgggtaagattagctcaaaattatcatcctgaattattagataatctttcatcagcaaaatcaccacaacaaatatttggtactgcatcaaaaacttactatccttcaatttcaggaatagctccagaagatgtttatacagttactatcatgccttgtaatgataaaaaatatgaagcagatattcctttcatggaaactaacagcttaagagatattgatgcatccttaactacaagagagcttgcaaaaatgattaaagatgcaaaaattaaatttgcagatcttgaagatggtgaagttgatcctgctatgggtacttacagtggtgctggagctatctttggtgcaaccggtggcgttatggaagctgcaataagatcagctaaagactttgctgaaaataaagaacttgaaaatgttgattacactgaagtaagaggctttaaaggcataaaagaagcggaagttgaaattgctggaaataaactaaacgttgctgttataaatggtgcttctaacttcttcgagtttatgaaatctggaaaaatgaacgaaaaacaatatcactttatagaagtaatggcttgccctggtggatgtataaatggtggaggtcaacctcacgtaaatgctcttgatagagaaaatgttgattacagaaaactaagagcatcagtattatacaaccaagataaaaatgttctttcaaagagaaagtcacatgataatccagctattattaaaatgtatgatagctactttggaaaaccaggtgaaggacttgctcacaaattactacacgtaaaatacacaaaagataaaaatgtttcaaaacatgaaactagagcggccgcttctagagctgcatataaagttactttggtaacaccaaccggtaatgtcgaatttcaatgtcctgatgacgtgtacattttagacgccgctgaggaagagggaatagatctaccatattcttgcagagcaggctcatgttccagttgcgccggtaagcttaaaactggaagcttgaaccaggatgaccaatctttcttagatgatgaccagatcgatgaaggctgggttctaacatgtgctgcataccctgtatcagacgtcaccattgaaactcataaggaggaagaacttacagccactagttaa

SEQ ID NO: 7 - protein, 788 aa, Artificial Sequence
  chemically synthesized
MGAAASRAAYKVTLVTPTGNVEFQCPDDVYILDAAEEEGIDLPYSCRAGSCSSCAGKLKTGSLNQDDQSFLDDDQIDEGWVLTCAAYPVSDVTIETHKEEELTATRKTIILNGNEVHTDKDITILELARENNVDIPTLCFLKDCGNFGKCGVCMVEVEGKGFRAACVAKVEDGMVINTESDEVKERIKKRVSMLLDKHEFKCGQCSRRENCEFLKLVIKTKAKASKPFLPEDKDALVDNRSKAIVIDRSKCVLCGRCVAACKQHTSTCSIQFIKKDGQRAVGTVDDVCLDDSTCLLCGQCVIACPVAALKEKSHIEKVQEALNDPKKHVIVAMAPSVRTAMGELFKMGYGKDVTGKLYTALRMLGFDKVFDINFGADMTIMEEATELLGRVKNNGPFPMFTSCCPAWVRLAQNYHPELLDNLSSAKSPQQIFGTASKTYYPSISGIAPEDVYTVTIMPCNDKKYEADIPFMETNSLRDIDASLTTRELAKMIKDAKIKFADLEDGEVDPAMGTYSGAGAIFGATGGVMEAAIRSAKDFAENKELENVDYTEVRGFKGIKEAEVEIAGNKLNVAVINGASNFFEFMKSGKMNEKQYHFIEVMACPGGCINGGGQPHVNALDRENVDYRKLRASVLYNQDKNVLSKRKSHDNPAIIKMYDSYFGKPGEGLAHKLLHVKYTKDKNVSKHETRAAYKVTLVTPTGNVEFQCPDDVYILDAAEEEGIDLPYSCRAGSCSSCAGKLKTGSLNQDDQSFLDDDQIDEGWVLTCAAYPVSDVTIETHKEEELTATS

SEQ ID NO: 8 - DNA, 2397 bp, Artificial Sequence
  chemically synthesized
atgggcgcggccgcttctagagcggccgcttctagagctgcatataaagttactttggtaacaccaaccggtaatgtcgaatttcaatgtcctgatgacgtgtacattttagacgccgctgaggaagagggaatagatctaccatattcttgcagagcaggctcatgttccagttgcgccggtaagcttaaaactggaagcttgaaccaggatgaccaatctttcttagatgatgaccagatcgatgaaggctgggttctaacatgtgctgcataccctgtatcagacgtcaccattgaaactcataaggaggaagaacttacagccactagaaaaacaataatcttaaatggcaatgaagtgcatacagataaagatattactatccttgagctagcaagagaaaataatgtagatatcccaacactctgctttttaaaggattgtggcaattttggaaaatgcggagtctgtatggtagaggtagaaggcaagggctttagagctgcttgtgttgccaaagttgaagatggaatggtaataaacacagaatccgatgaagtaaaagaacgaatcaaaaaaagagtttcaatgcttcttgataagcatgaatttaaatgtggacaatgttctagaagagaaaattgtgaattccttaaacttgtaataaagacaaaagcaaaagcttcaaaaccatttttaccagaagataaggatgctctagttgataatagaagtaaggctattgtaattgacagatcaaaatgtgtactatgcggtagatgcgtagctgcatgtaaacagcacacaagcacttgctcaattcaatttattaaaaaagatggacaaagggctgttggaactgttgatgatgtttgtcttgatgactcaacatgcttattatgcggtcagtgtgtaatcgcttgtcctgttgctgctttaaaagaaaaatcccatatagaaaaagttcaagaagctcttaatgaccctaaaaaacatgtcattgttgcaatggctccatcagtaagaactgctatgggcgaattattcaaaatgggatatggaaaagatgtaacaggaaaactatatactgcacttagaatgttaggctttgataaagtatttgatataaactttggtgcagatatgactataatggaagaagctactgaacttttaggcagagttaaaaataatggcccattccctatgtttacatcttgctgtcctgcatgggtaagattagctcaaaattatcatcctgaattattagataatctttcatcagcaaaatcaccacaacaaatatttggtactgcatcaaaaacttactatccttcaatttcaggaatagctccagaagatgtttatacagttactatcatgccttgtaatgataaaaaatatgaagcagatattcctttcatggaaactaacagcttaagagatattgatgcatccttaactacaagagagcttgcaaaaatgattaaagatgcaaaaattaaatttgcagatcttgaagatggtgaagttgatcctgctatgggtacttacagtggtgctggagctatctttggtgcaaccggtggcgttatggaagctgcaataagatcagctaaagactttgctgaaaataaagaacttgaaaatgttgattacactgaagtaagaggctttaaaggcataaaagaagcggaagttgaaattgctggaaataaactaaacgttgctgttataaatggtgcttctaacttcttcgagtttatgaaatctggaaaaatgaacgaaaaacaatatcactttatagaagtaatggcttgccctggtggatgtataaatggtggaggtcaacctcacgtaaatgctcttgatagagaaaatgttgattacagaaaactaagagcatcagtattatacaaccaagataaaaatgttctttcaaagagaaagtcacatgataatccagctattattaaaatgtatgatagctactttggaaaaccaggtgaaggacttgctcacaaattactacacgtaaaatacacaaaagataaaaatgtttcaaaacatgaaactagagcggccgcttctagagctgcatataaagttactttggtaacaccaaccggtaatgtcgaatttcaatgtcctgatgacgtgtacattttagacgccgctgaggaagagggaatagatctaccatattcttgcagagcaggctcatgttccagttgcgccggtaagcttaaaactggaagcttgaaccaggatgaccaatctttcttagatgatgaccagatcgatgaaggctgggttctaacatgtgctgcataccctgtatcagacgtcaccattgaaactcataaggaggaagaacttacagccactagttaa

SEQ ID NO: 9 - protein, 709 aa, Artificial Sequence
  chemically synthesized
MGAAASRAAYKVTLVTPTGNVEFQCPDDVYILDAAEEEGIDLPYSCRAGSCSSCAGKLKTGSLNQDDQSFLDDDQIDEGWVLTCAAYPVSDVTIETHKEEELTATRGGGGSGGGGSGGGGSGGGGSKTIILNGNEVHTDKDITILELARENNVDIPTLCFLKDCGNFGKCGVCMVEVEGKGFRAACVAKVEDGMVINTESDEVKERIKKRVSMLLDKHEFKCGQCSRRENCEFLKLVIKTKAKASKPFLPEDKDALVDNRSKAIVIDRSKCVLCGRCVAACKQHTSTCSIQFIKKDGQRAVGTVDDVCLDDSTCLLCGQCVIACPVAALKEKSHIEKVQEALNDPKKHVIVAMAPSVRTAMGELFKMGYGKDVTGKLYTALRMLGFDKVFDINFGADMTIMEEATELLGRVKNNGPFPMFTSCCPAWVRLAQNYHPELLDNLSSAKSPQQIFGTASKTYYPSISGIAPEDVYTVTIMPCNDKKYEADIPFMETNSLRDIDASLTTRELAKMIKDAKIKFADLEDGEVDPAMGTYSGAGAIFGATGGVMEAAIRSAKDFAENKELENVDYTEVRGFKGIKEAEVEIAGNKLNVAVINGASNFFEFMKSGKMNEKQYHFIEVMACPGGCINGGGQPHVNALDRENVDYRKLRASVLYNQDKNVLSKRKSHDNPAIIKMYDSYFGKPGEGLAHKLLHVKYTKDKNVSKHETS

SEQ ID NO: 10 - DNA, 2145 bp, Artificial Sequence
  chemically synthesized
atgggcgcggccgcttctagagcggccgcttctagagctgcatataaagttactttggtaacaccaaccggtaatgtcgaatttcaatgtcctgatgacgtgtacattttagacgccgctgaggaagagggaatagatctaccatattcttgcagagcaggctcatgttccagttgcgccggtaagcttaaaactggaagcttgaaccaggatgaccaatctttcttagatgatgaccagatcgatgaaggctgggttctaacatgtgctgcataccctgtatcagacgtcaccattgaaactcataaggaggaagaacttacagccactagaggtggtggaggatcaggtggtggaggatcaggtggtggaggatcaggtggtggaggatcaaaaacaataatcttaaatggcaatgaagtgcatacagataaagatattactatccttgagctagcaagagaaaataatgtagatatcccaacactctgctttttaaaggattgtggcaattttggaaaatgcggagtctgtatggtagaggtagaaggcaagggctttagagctgcttgtgttgccaaagttgaagatggaatggtaataaacacagaatccgatgaagtaaaagaacgaatcaaaaaaagagtttcaatgcttcttgataagcatgaatttaaatgtggacaatgttctagaagagaaaattgtgaattccttaaacttgtaataaagacaaaagcaaaagcttcaaaaccatttttaccagaagataaggatgctctagttgataatagaagtaaggctattgtaattgacagatcaaaatgtgtactatgcggtagatgcgtagctgcatgtaaacagcacacaagcacttgctcaattcaatttattaaaaaagatggacaaagggctgttggaactgttgatgatgtttgtcttgatgactcaacatgcttattatgcggtcagtgtgtaatcgcttgtcctgttgctgctttaaaagaaaaatcccatatagaaaaagttcaagaagctcttaatgaccctaaaaaacatgtcattgttgcaatggctccatcagtaagaactgctatgggcgaattattcaaaatgggatatggaaaagatgtaacaggaaaactatatactgcacttagaatgttaggctttgataaagtatttgatataaactttggtgcagatatgactataatggaagaagctactgaacttttaggcagagttaaaaataatggcccattccctatgtttacatcttgctgtcctgcatgggtaagattagctcaaaattatcatcctgaattattagataatctttcatcagcaaaatcaccacaacaaatatttggtactgcatcaaaaacttactatccttcaatttcaggaatagctccagaagatgtttatacagttactatcatgccttgtaatgataaaaaatatgaagcagatattcctttcatggaaactaacagcttaagagatattgatgcatccttaactacaagagagcttgcaaaaatgattaaagatgcaaaaattaaatttgcagatcttgaagatggtgaagttgatcctgctatgggtacttacagtggtgctggagctatctttggtgcaaccggtggcgttatggaagctgcaataagatcagctaaagactttgctgaaaataaagaacttgaaaatgttgattacactgaagtaagaggctttaaaggcataaaagaagcggaagttgaaattgctggaaataaactaaacgttgctgttataaatggtgcttctaacttcttcgagtttatgaaatctggaaaaatgaacgaaaaacaatatcactttatagaagtaatggcttgccctggtggatgtataaatggtggaggtcaacctcacgtaaatgctcttgatagagaaaatgttgattacagaaaactaagagcatcagtattatacaaccaagataaaaatgttctttcaaagagaaagtcacatgataatccagctattattaaaatgtatgatagctactttggaaaaccaggtgaaggacttgctcacaaattactacacgtaaaatacacaaaagataaaaatgtttcaaaacatgaaactagttaa

SEQ ID NO: 11 - protein, 497 aa, Artificial Sequence
  chemically synthesized
MSALVLKPCAAVSIRGSSCRARQVAPRAPLAASTVRVALATLEAPARRLGNVACAAAAPAAEAPLSHVQQALAELAKPKDDPTRKHVCVQVAPAVRVAIAETLGLAPGATTPKQLAEGLRRLGFDEVFDTLFGADMTIMEEGSELLHRLTEHLEAHPHSDEPFPMFTSCCPGWIAMLEKSYPDLIPYVSSCKSPQMMLAAMVKSYLAEKKGIAPKDMVMVSIMPCTRKQSEADRDWFCVDADPTLRQLDHVITTVELGNIFKERGINLAELPEGEWDNPMGVGSGAGVLFGTTGGVMEAALRTAYELFTGTPLPRLSLSEVRGMDGIKETNITMVPAPGSKFEELLKHRAAARAEAAAHGTPGPLAWDGGAGFTSEDGRGGITYRVAVANGLGNAKKLITKMQAGEAKYDFVEIMACPAGCVGGGGQPRSTDKAITQKRQAALYNLDEKSTLRRSHENPSIREMYDTYFGEPLGHKAHELLHTHYVAGGVEEKDEKK

SEQ ID NO: 12 - protein, 574 aa, Clostridium pasteurianum
MKTIIINGVQFNTDEDTTILKFARDNNIDISALCFLNNCNNDINKCEICTVEVEGTGLVTACDTLIEDGMIINTNSDAVNEKIKSRISQLLDIHEFKCGPCNRRENCEFLKLVIKYKARASKPFLPKDKTEYVDERSKSLTVDRTKCLLCGRCVNACGKNTETYAMKFLNKNGKTIIGAEDEKCFDDTNCLLCGQCIIACPVAALSEKSHMDRVKNALNAPEKHVIVAMAPSVRASIGELFNMGFGVDVTGKIYTALRQLGFDKIFDINFGADMTIMEEATELVQRIENNGPFPMFTSCCPGWVRQAENYYPELLNNLSSAKSPQQIFGTASKTYYPSISGLDPKNVFTVTVMPCTSKKFEADRPQMEKDGLRDIDAVITTRELAKMIKDAKIPFAKLEDSEADPAMGEYSGAGAIFGATGGVMEAALRSAKDFAENAELEDIEYKQVRGLNGIKEAEVEINNNKYNVAVINGASNLFKFMKSGMINEKQYHFIEVMACHGGCVNGGGQPHVNPKDLEKVDIKKVRASVLYNQDEHLSKRKSHENTALVKMYQNYFGKPGEGRAHEILHFKYKK

SEQ ID NO: 13 - protein, 574 aa, Artificial Sequence
  chemically synthesized
MKTIIINGVQFNTDEDTTILKFARDNNIDISALCFLNNCNNDINKCEICTVEVEGTGLVTACDTLIEDGMIINTNSDAVNEKIKSRISQLLDIHEFKCGPCNRRENCEFLKLVIKYKARASKPFLPKDKTEYVDERSKSLTVDRTKCLLCGRCVNACGKNTETYAMKFLNKNGKTIIGAEDEKCFDDTNCLLCGQCIIACPVAALSEKSHMDRVKNALNAPEKHVIVAMAPSVRASIGELFNMGFGVDVTGKIYTALRQLGFDKIFDINFGADMVIMEEATELIQRIENNGPFPMFTSCCPGWVRQAENYYPELLNNLSSAKSPQQIFGTASKTYYPSISGLDPKNVFTVTVMPCTSKKFEADRPQMEKDGLRDIDAVITTRELAKMIKDAKIPFAKLEDSEADPAMGEYSGAGAIFGATGGVMEAALRSVKDFLENAELEDIEYKQVRGLNGIKEAEVEINNNKYNVAVINGASNLFKFMKSGMINEKQYHYIEVMACHGGCVNGGGQPHVNPKDLEKVDIKKVRASVLYNQDEHLSKRKSHENTALVKMYQNYFGKPGEGRAHEILHFKYKK

SEQ ID NO: 14 - protein, 574 aa, Artificial Sequence
  chemically synthesized
MKTIIINGVQFNTDEDTTILKFARDNNIDISALCFLNNCNNDINKCEICTVEVEGTGLVTACDTLIEDGMIINTNSDAVNEKIKSRISQLLDIHEFKCGPCNRRENCEFLKLVIKYKARASKPFLPKDKTEYVDERSKSLTVDRTKCLLCGRCVNACGKNTETYAMKFLNKNGKTIIGAEDEKCFDDTNCLLCGQCIIACPVAALSEKSHMDRVKNALNAPEKHVIVAMAPSVRASIGELFNMGFGVDVTGKIYTALRQLGFDKIFDINFGADMVIMEEATELIQRIEGNGPFPMFTSCCPGWVRQAENYYPELLNNLSSAKSPQQIFGTASKTYYPSISGLDPKNVFTVTVMPCTSKKFEADRPQMEKDGLRDIDAVITTRELAKMIKDAKIPFAKLEDSEADPAMGEYSGAGAIFGATGGVMEAALRSVKDFLENAELEDIEYKQVRGLNGIKEAEVEIRNNKYNFAVINGASNLFKFMKSGMINEKQYHYIEVMACHGGCVNGGGQPHVNPKDLEKVDIKKVRASVLYNQDEHLSKRKSHENTALVKMYQNYFGKPGEGRAHEILHFKYKK

SEQ ID NO: 15 - DNA, 40 bp, Artificial Sequence
  chemically synthesized
atgggcccactagtgtcgaaacattttatgaagtcatgcg

SEQ ID NO: 16 - DNA, 32 bp, Artificial Sequence
  chemically synthesized
ataagctttctagatcaagatcgtttccccgc

SEQ ID NO: 17 - protein, 566 aa, Artificial Sequence
  chemically synthesized
MVETFYEVMRRQGISRRSFLKYCSLTATSLGLGPSFLPQIAHAMETKPRTPVLWLHGLECTCCSESFIRSAHPLAKDVVLSMISLDYDDTLMAAAGHQAEAILEEIMTKYKGNYILAVEGNPPLNQDGMSCIIGGRPFIEQLKYVAKDAKAIISWGSCASWGCVQAAKPNPTQATPVHKVITDKPIIKVPGCPPIAEVMTGVITYMLTFDRIPELDRQGRPKMFYSQRIHDKCYRRPHFDAGQFVEEWDDESARKGFCLYKMGCKGPTTYNACSTTRWNEGTSFPIQSGHGCIGCSEDGFWDKGSFYDRGGGGSGGGGSGGGGSGGGGSGGGGSGGGGSGGGGSAAYKVTLVTPTGNVEFQCPDDVYILDAAEEEGIDLPYSCRAGSCSSCAGKLKTGSLNQDDQSFLDDDQIDEGWVLTCAAYPVSDVTIETHKEEELTAGGGGSGGGGSGGGGSGGGGSGGGGSGGGGSGGGGSGGGGSGGGGSGGGGSMAIARGDKVRILRPESYWFNEVGTVASVDQSGIKYPVVVRFEKVNYNGFSGSDGGVNTNNFAEAELQVVAAAAKK

SEQ ID NO: 18 - protein, 645 aa, Thermotoga maritima
MKIYVDGREVIINDNERNLLEALKNVGIEIPNLCYLSEASIYGACRMCLVEINGQITTSCTLKPYEGMKVKTNTPEIYEMRRNILELILATHNRDCTTCDRNGSCKLQKYAEDFGIRKIRFEALKKEHVRDESAPVVRDTSKCILCGDCVRVCEEIQGVGVIEFAKRGFESVVTTAFDTPLIETECVLCGQCVAYCPTGALSIRNDIDKLIEALESDKIVIGMIAPAVRAAIQEEFGIDEDVAMAEKLVSFLKTIGFDKVFDVSFGADLVAYEEAHEFYERLKKGERLPQFTSCCPAWVKHAEHTYPQYLQNLSSVKSPQQALGTVIKKIYARKLGVPEEKIFLVSFMPCTAKKFEAEREEHEGIVDIVLTTRELAQLIKMSRIDINRVEPQPFDRPYGVSSQAGLGFGKAGGVFSCVLSVLNEEIGIEKVDVKSPEDGIRVAEVTLKDGTSFKGAVIYGLGKVKKFLEERKDVEIIEVMACNYGCVGGGGQPYPNDSRIREHRAKVLRDTMGIKSLLTPVENLFLMKLYEEDLKDEHTRHEILHTTYRPRRRYPEKDVEILPVPNGEKRTVKVCLGTSCYTKGSYEILKKLVDYVKENDMEGKIEVLGTFCVENCGASPNVIVDDKIIGGATFEKVLEELSKNG

SEQ ID NO: 19 - DNA, 1938 bp, Thermotoga maritima
atgaaaatttacgttgatggaagagaagttatcataaatgacaacgagcgtaacctccttgaagcgctgaagaacgtggggatagagattccgaatctgtgttatctttcggaggcttctatatatggagcctgtagaatgtgtcttgtggagatcaacggtcagatcaccacttcctgtaccctgaaaccgtacgaaggtatgaaggtaaaaacgaacacccccgaaatatacgaaatgagaagaaacatcctcgaactcatcctcgcaactcacaacagggactgcaccacctgcgatagaaacggaagctgtaaacttcagaagtacgctgaagacttcggcataagaaagatcagattcgaggctctcaagaaagaacacgtcagggacgaatccgctccggtagtgagagatacatccaagtgtattctctgcggtgactgtgttcgcgtgtgtgaagaaattcagggagtcggtgttatcgagttcgcaaagcgcggttttgaaagcgttgtgacaaccgcttttgatactcccctcatagagacggagtgtgtgctctgcggacagtgtgtagcctactgtccaacgggagctctgagcatcagaaacgacatagacaagttgatcgaagctctcgaaagcgataagatcgtgataggaatgatcgcacctgcggtgagggctgcgattcaggaagagtttggaatagacgaagacgtcgcaatggcggaaaaactcgtctctttcctgaaaacgataggcttcgataaagtcttcgatgtgtcgttcggagcagaccttgtcgcctacgaagaagcccacgagttctatgaaagactcaaaaaaggagaaagacttccacagttcacctcatgctgtcccgcatgggtgaagcacgctgagcacacctatcctcagtaccttcagaatctctcgagcgtgaaatcacctcaacaggcactcggtacggtgataaagaagatctacgcaagaaaactcggtgttcccgaagaaaagatcttcctcgtttcgttcatgccgtgtaccgctaaaaagttcgaagcagaaagagaagaacacgaaggaatcgttgacattgtcctcacaacaagggaactcgctcaactcatcaagatgagcagaatagacataaacagagtagaaccccagccgttcgacagaccttacggagtgtcttcgcaggcgggtctcggttttggaaaagccggtggggtcttctcctgtgttctttctgtgttgaacgaggaaatcggcatagaaaaagtcgatgtaaaatctccggaagatggcatcagggtagcggaagttacactcaaagatggtacgtctttcaaaggagctgtcatatacggtcttggtaaggtgaagaagttcctcgaagaaagaaaagacgtggagattatcgaagtaatggcctgtaactacggatgtgtgggtgggggaggacagccttacccgaacgattccagaatcagagaacacagggcaaaagtgctaagagacaccatgggaataaaatctctcctcacacccgtggaaaacctctttctcatgaaactctacgaggaagatctgaaagacgaacacacaagacacgaaattctccacaccacctaccgaccgaggagaagatacccggaaaaagatgtggaaatactgcccgttccaaacggcgaaaagagaacggtgaaagtctgtcttggaacctcctgttacacgaaagggtcttacgagatattgaaaaagcttgtcgactacgtcaaagagaacgatatggaaggaaagatagaagtgctgggaacgttctgcgtggaaaactgcggtgcttctccaaacgtgatcgtggatgataaaatcataggtggtgccacttttgagaaggtgctggaggagctttcgaaaaatggctga

SEQ ID NO: 20 - protein, 582 aa, Clostridium acetobutylicum
MKTIILNGNEVHTDKDITILELARENNVDIPTLCFLKDCGNFGKCGVCMVEVEGKGFRAACVAKVEDGMVINTESDEVKERIKKRVSMLLDKHEFKCGQCSRRENCEFLKLVIKTKAKASKPFLPEDKDALVDNRSKAIVIDRSKCVLCGRCVAACKQHTSTCSIQFIKKDGQRAVGTVDDVCLDDSTCLLCGQCVIACPVAALKEKSHIEKVQEALNDPKKHVIVAMAPSVRTAMGELFKMGYGKDVTGKLYTALRMLGFDKVFDINFGADMTIMEEATELLGRVKNNGPFPMFTSCCPAWVRLAQNYHPELLDNLSSAKSPQQIFGTASKTYYPSISGIAPEDVYTVTIMPCNDKKYEADIPFMETNSLRDIDASLTTRELAKMIKDAKIKFADLEDGEVDPAMGTYSGAGAIFGATGGVMEAAIRSAKDFAENKELENVDYTEVRGFKGIKEAEVEIAGNKLNVAVINGASNFFEFMKSGKMNEKQYHFIEVMACPGGCINGGGQPHVNALDRENVDYRKLRASVLYNQDKNVLSKRKSHDNPAIIKMYDSYFGKPGEGLAHKLLHVKYTKDKNVSKHE

SEQ ID NO: 21 - DNA, 1749 bp, Clostridium acetobutylicum
atgaaaacaataatcttaaatggcaatgaagtgcatacagataaagatattactatccttgagctagcaagagaaaataatgtagatatcccaacactctgctttttaaaggattgtggcaattttggaaaatgcggagtctgtatggtagaggtagaaggcaagggctttagagctgcttgtgttgccaaagttgaagatggaatggtaataaacacagaatccgatgaagtaaaagaacgaatcaaaaaaagagtttcaatgcttcttgataagcatgaatttaaatgtggacaatgttctagaagagaaaattgtgaattccttaaacttgtaataaagacaaaagcaaaagcttcaaaaccatttttaccagaagataaggatgctctagttgataatagaagtaaggctattgtaattgacagatcaaaatgtgtactatgcggtagatgcgtagctgcatgtaaacagcacacaagcacttgctcaattcaatttattaaaaaagatggacaaagggctgttggaactgttgatgatgtttgtcttgatgactcaacatgcttattatgcggtcagtgtgtaatcgcttgtcctgttgctgctttaaaagaaaaatcccatatagaaaaagttcaagaagctcttaatgaccctaaaaaacatgtcattgttgcaatggctccatcagtaagaactgctatgggcgaattattcaaaatgggatatggaaaagatgtaacaggaaaactatatactgcacttagaatgttaggctttgataaagtatttgatataaactttggtgcagatatgactataatggaagaagctactgaacttttaggcagagttaaaaataatggcccattccctatgtttacatcttgctgtcctgcatgggtaagattagctcaaaattatcatcctgaattattagataatctttcatcagcaaaatcaccacaacaaatatttggtactgcatcaaaaacttactatccttcaatttcaggaatagctccagaagatgtttatacagttactatcatgccttgtaatgataaaaaatatgaagcagatattcctttcatggaaactaacagcttaagagatattgatgcatccttaactacaagagagcttgcaaaaatgattaaagatgcaaaaattaaatttgcagatcttgaagatggtgaagttgatcctgctatgggtacttacagtggtgctggagctatctttggtgcaaccggtggcgttatggaagctgcaataagatcagctaaagactttgctgaaaataaagaacttgaaaatgttgattacactgaagtaagaggctttaaaggcataaaagaagcggaagttgaaattgctggaaataaactaaacgttgctgttataaatggtgcttctaacttcttcgagtttatgaaatctggaaaaatgaacgaaaaacaatatcactttatagaagtaatggcttgccctggtggatgtataaatggtggaggtcaacctcacgtaaatgctcttgatagagaaaatgttgattacagaaaactaagagcatcagtattatacaaccaagataaaaatgttctttcaaagagaaagtcacatgataatccagctattattaaaatgtatgatagctactttggaaaaccaggtgaaggacttgctcacaaattactacacgtaaaatacacaaaagataaaaatgtttcaaaacatgaataa

SEQ ID NO: 22 - protein, 574 aa, Unknown
  Clostridium saccharobutylicum species
MINIVIDEKTIQVQENTTVIQAALANGIDIPSLCYLNECGNVGKCGVCAVEIEGKNNLALACITKVEEGMVVKTNSEKVQERVKMRVATLLDKHEFKCGPCPRRENCEFLKLVIKTKAKANKPFVVEDKSQYIDIRSKSIVIDRTKCVLCGRCEAACKTKTGTGAISICKSESGRIVQATGGKCFDDTNCLLCGQCVAACPVGALTEKTHVDRVKEALEDPNKHVIVAMAPSIRTSMGELFKLGYGVDVTGKLYASMRALGFDKVFDINFGADMTIMEEATEFIERVKNNGPFPMFTSCCPAWVRQVENYYPEFLENLSSAKSPQQIFGAASKTYYPQISGISAKDVFTVTIMPCTAKKFEADREEMYNEGIKNIDAVLTTRELAKMIKDAKINFANLEDEQADPAMGEYTGAGVIFGATGGVMEAALRTAKDFVEDKDLTDIEYTQIRGLQGIKEATVEIGGENYNVAVINGAANLAEFMNSGKILEKNYHFIEVMACPGGCVNGGGQPHVSAKEREKVDVRTVRASVLYNQDKNLEKRKSHKNTALLNMYYDYMGAPGQGKAHELLHLKYNK

SEQ ID NO: 23 - DNA, 1725 bp, Unknown
  Clostridium saccharobutylicum species
atgataaacatagtaattgatgaaaaaactattcaagtacaggaaaatactacagttatacaagctgccctagcaaatgggatagatataccaagtttatgctatcttaatgagtgtggtaatgttggaaagtgtggagtgtgtgcagtagaaatagaaggaaaaaataacttagcacttgcatgtataacaaaagttgaagaaggtatggtagtaaaaacaaactcagaaaaagtacaagaaagagttaaaatgagagttgctactttgcttgataagcatgaatttaaatgtggaccttgtccaagaagagaaaattgcgaatttttaaagttagttataaaaacaaaagctaaggctaacaagccttttgtggttgaagacaaatcacaatacatagatattagaagtaaatcaattgtaatagacagaactaagtgtgtgctatgcggaagatgtgaagcagcatgtaaaacaaagacaggtacaggagctatttcaatttgtaagagtgaatcaggaagaatagtgcaagcaacaggcggaaagtgctttgatgatacaaattgtttattatgtggacaatgcgttgcagcatgtccagtaggagctttaactgaaaaaacacacgttgatagagttaaagaagcattagaagatcctaataagcatgtaatagttgctatggcaccatcaatcagaacttctatgggagagttatttaaattaggctatggggttgatgtaactggaaaattatatgcttcaatgagagcattaggatttgataaggtatttgatattaactttggggctgatatgacaataatggaagaagcaacagagtttattgaaagagttaaaaataatggaccattcccaatgtttacttcatgttgtccggcatgggttagacaagtggaaaattattacccagaatttttagaaaacttatcatcagctaaatcaccacaacaaatatttggtgcagcaagcaaaacatactatcctcaaatatcaggtataagtgctaaagatgtatttactgttacaataatgccttgtacagcaaagaaatttgaggctgatagagaagaaatgtataatgagggaattaaaaatatagatgcagtacttactacaagagaattagcaaaaatgattaaagatgcaaagattaattttgctaatttagaagacgaacaagctgatccagcaatgggagaatacactggggctggagttatattcggagctacaggtggagttatggaagcagcacttagaactgctaaggatttcgttgaagataaagatttaactgatatagaatatacacaaataagaggattacaaggaataaaagaggctacagtagaaattggtggagaaaattataacgtagctgtaattaatggtgcagcaaacttagctgaattcatgaatagcggtaaaatccttgaaaagaactatcattttattgaagtaatggcttgcccaggcggatgtgtaaatggtggaggacaaccacacgtaagtgcaaaggaaagagaaaaagtagatgttagaactgtaagagcatctgttttatataaccaagataaaaatttagagaagagaaaatcacataaaaatacagcattattaaatatgtactatgattatatgggagctccaggacaaggaaaagctcatgaattattacacttaaaatacaataaataa

SEQ ID NO: 24 - protein, 497 aa, Chlamydomonas reinhardtii
MSALVLKPCAAVSIRGSSCRARQVAPRAPLAASTVRVALATLEAPARRLGNVACAAAAPAAEAPLSHVQQALAELAKPKDDPTRKHVCVQVAPAVRVAIAETLGLAPGATTPKQLAEGLRRLGFDEVFDTLFGADLTIMEEGSELLHRLTEHLEAHPHSDEPLPMFTSCCPGWIAMLEKSYPDLIPYVSSCKSPQMMLAAMVKSYLAEKKGIAPKDMVMVSIMPCTRKQSEADRDWFCVDADPTLRQLDHVITTVELGNIFKERGINLAELPEGEWDNPMGVGSGAGVLFGTTGGVMEAALRTAYELFTGTPLPRLSLSEVRGMDGIKETNITMVPAPGSKFEELLKHRAAARAEAAAHGTPGPLAWDGGAGFTSEDGRGGITLRVAVANGLGNAKKLITKMQAGEAKYDFVEIMACPAGCVGGGGQPRSTDKAITQKRQAALYNLDEKSTLRRSHENPSIRELYDTYLGEPLGHKAHELLHTHYVAGGVEEKDEKK

SEQ ID NO: 25 - protein, 574 aa, Clostridium pasteurianum; identical to SEQ ID NO: 12

SEQ ID NO: 26 - protein, 606 aa, Desulfovibrio gigas
MNAFINGKEVRCEPGRTILEAARENGHFIPTLCELADIGHAPGTCRVCLVEIWRDKEAGPQIVTSCTTPVEEGMRIFTRTPEVRRMQRLQVELLLADHDHDCAACARHGDCELQDVAQFVGLTGTRHHFPDYARSRTRDVSSPSVVRDMGKCIRCLRCVAVCRNVQGVDALVVTGNGIGTEIGLRHNRSQSASDCVGCGQCTLVCPVGALAGRDDVERVIDYLYDPEIVTVFQFAPAVRVGLGEEFGLPPGSSVEGQVPTALRLLGADVVLDTNFAADLVIMEEGTELLQRLRGGAKLPLFTSCCPGWVNFAEKHLPDILPHVSTTRSPQQCLGALAKTYLARTMNVAPERMRVVSLMPCTAKKEEAARPEFRRDGVRDVDAVLTTREFARLLRREGIDLAGLEPSPCDDPLMGRATGAAVIFGTTGGVMEAALRTVYHVLNGKELAPVELHALRGYENVREAVVPLGEGNGSVKVAVVHGLKAARQMVEAVLAGKADHVFVEVMACPGGCMDGGGQPRSKRAYNPNAQARRAALFSLDAENALRQSHNNPLIGKVYESFLGEPCSNLSHRLLHTRYGDRKSEVAYTMRDIWHEMTLGRRVRGDSD

SEQ ID NO: 27 - protein, 407 aa, Unknown
  [FeFe]-hydrogenase sequence from Sargasso Sea Database
SSPAMIRDMTKCIRCFRCVDVCREVQDVDALVIKGAGSETQIGLKGGDSQVDSDCVTCGQCVMVCPVGALAERDDTETVIDYIYDPDVTTVFQFAPAIRVGLGEEFGMEPGTNVEGNIIAALRKLGGDIILDTNFAADVVIMEEGTELIHQLKENKRPTFTSCCPSWINFAEKNYPELLPNLSTTKSPQQVLGTLAKTYLAEKMEIDPKKMKVISIMPCTAKKDEITRPQLQFDGEMPEVDTVLTVREFVRLLHREGIDFVNLEPSSFDNPYMSEYSGAGVIFGTTGGVMEAAIRTVYYVLNGKELEGTVVEQLRGFEGMRAAKVDLGPEVGTVKVAMCHGLKETRQICESVMAGDADFDFIEIMACPGGCVDGGGNLRSKKSYLPHALKRRDTLFQIDANATARQS

SEQ ID NO: 28 - protein, 301 aa, Unknown
  [FeFe]-hydrogenase sequence from Sargasso Sea Database
KIVTGQLVASIKKMGFDYVFDVNLGADLTTYEEAKELVHWLKSGKDRPMFTSCCPGWVKFVEFFYPEFVSHLTTTKSPVICSSSIIKTYFADILKKDPRDIINITIMPCTAKKHEANLNRHKIDLGWCIERLDLKNIEQVCKNRQNLQGIKIPAVDYVLTTREYAYLLHKHKIDLPNLKPEDADKPLNIYSGAGAIYGATGGVMESALRSAYYFLNKNNVKTQQVAHLQASHIEFEQARGMDGIKTAQVKVGGEKLNIAVVNGLCNARKLLEDIKSKKIEFDYVEVMACPGGCIGGGGQPV

SEQ ID NO: 29 - protein, 298 aa, Unknown
  [FeFe]-hydrogenase sequence from Sargasso Sea Database
LLERIKKNEILPQFTSCCPAWVKFVEHYYPDLIPYLSTAKSPHQMLGATIKAFYAEKYGTTAEKIVNVSVMPCTAKKFERQRAEMNSNDGLMDVDFILTTRELATMIRKTAIDFASLPDEEFDSLAQGSGAGDIFGATGGVMEAALRTAYEVQTGNKLNKLEFDQIRGLQGVKEGHIKMDGKEVWFAVVSGLNNVKPIIEEVLAGKSKYHFIEVMTCPGGCIGGGGQPIPTNQEIVEKRMHGIYASDKNKAIRRSYENPQIKALYSEFFGNPLSEKAEKYLHTHFIKRGKYNKSSKEK

SEQ ID NO: 30 - protein, 288 aa, Unknown
  [FeFe]-hydrogenase sequence from Sargasso Sea Database
GFDKVFDVNMGADITTMVEAGELIERLESGEHLPMFTSCCPGWVKYVEFYHPELIPNLTTSRSPQIHSGGAYKTWWAKKVSIDPKDIVIVSVMPCTSKKYEAHHDKLNINGLRPVDYSLTTREIAQMIRNHKIDFAKLKPSEVDAEGLYSGAAVIYGASGGVMESALRTAHFLVTGKELEKIDLKEVRGYKGIKKATITIGDLKLKVAVVATPKNIQHILRELKLNPHAYDYIEFMSCPGGCLGGGGQPNPSSKRIVEQRIKGIYAIDKKMQMRRAHENPVMQDSLNM

SEQ ID NO: 31 - protein, 458 aa, Unknown
  [FeFe]-hydrogenase sequence from Sargasso Sea Database
DTMVNLSINGMPLKVPEGTTILEAAKQLNFRIPVLCHHDDLCVAGNCRVCVVEQLGGKALLAACATPVSEGMQILTNSLKVRSARKHVIELLLSEHNADCTKCYKNGKCELQNLANEFSVGDHLFLDLTDIKDYTVDKFSPSIQKDDSKCIRCQRCVRTCQQLQGVNALTVAFKGDRQKISTFEDLSMSEVICTNCGQCINRCPTGALVERTYLDEVWDAILDPDKHVVVQTAPAVRVGLGEELGLEPGNRVTGKMVAALKRLGFDSVLDTDFTADLTIMEEGTELLTRLKKALVEKDDQVAIPMTTSCSPGWVKFIEHTFPEYLPNVSTCKSPQQMFGALAKTYYAQVRGIEPRDIVSVSIMPCTAKKYEANRPEMRSSGYKDVDYVLTTRELARMIKQAGVDFNKLKEDRYDSIMGTSTGAAVIFGATGGVMEAALRTAYEIVTGREVPFEDLNIN

SEQ ID NO: 32 - protein, 264 aa, Desulfovibrio gigas
LTAKKRPSVVYLHNAECTGCSESVLRTVDPYVDELILDVISMDYHETLMAGAGHAVEEALHEAIKGDFVCVIEGGIPMGDGGYWGKVGGRNMYDICAEVAPKAKAVIAIGTCATYGGVQAAKPNPTGTVGVNEALGKLGVKAINIAGCPPNPMNFVGTVVHLLTKGMPELDKQGRPVMFFGETVHDNCPRLKHFEAGEFATSFGSPEAKKGYCLYELGCKGPDTYNNCPKQLFNQVNWPVQAGHPCIACSEPNFWDLYSPFYSA

SEQ ID NO: 33 - protein, 266 aa, Desulfovibrio desulfuricans
ALTGSRPSVVYLHAAECTGCSEALLRTYQPFIDTLILDTISLDYHETIMAAAGEAAEEALQAAVNGPDGFICLVEGAIPTGMDNKYGYIAGHTMYDICKNILPKAKAVVSIGTCACYGGIQAAKPNPTAAKGINDCYADLGVKAINVPGCPPNPLNMVGTLVAFLKGQKIELDEVGRPVMFFGQSVHDLCERRKHFDAGEFAPSFNSEEARKGWCLYDVGCKGPETYNNCPKVLFNETNWPVAAGHPCIGCSEPNFWDDMTPFYQN

SEQ ID NO: 34 - protein, 360 aa, Ralstonia eutropha
MVETFYEVMRRQGISRRSFLKYCSLTATSLGLGPSFLPQIAHAMETKPRTPVLWLHGLECTCCSESFIRSAHPLAKDVVLSMISLDYDDTLMAAAGHQAEAILEEIMTKYKGNYILAVEGNPPLNQDGMSCIIGGRPFIEQLKYVAKDAKAIISWGSCASWGCVQAAKPNPTQATPVHKVITDKPIIKVPGCPPIAEVMTGVITYMLTFDRIPELDRQGRPKMFYSQRIHDKCYRRPHFDAGQFVEEWDDESARKGFCLYKMGCKGPTTYNACSTTRWNEGTSFPIQSGHGCIGCSEDGFWDKGSFYDRLTGISQFGVEANADKIGGTASVVVGAAVTAHAAASAIKRASKKNETSGSEH

SEQ ID NO: 35 - protein, 283 aa, Desulfovibrio baculatus
MTEGAKKAPVIWVQGQGCTGCSVSLLNAVHPRIKEILLDVISLEFHPTVMASEGEMALAHMYEIAEKFNGNFFLLVEGAIPTAKEGRYCIVGETLDAKAHHHEVTMMELIRDLAPKSLATVAVGTCSAYGGIPAAEGNVTGSKSVRDFFADEKIEKLLVNVPGCPPHPDWMVGTLVAAWSHVLNPTEHPLPELDDDGRPLLFFGDNIHENCPYLDKYDNSEFAETFTKPGCKAELGCKGPSTYADCAKRRWNNGINWCVENAVCIGCVEPDFPDGKSPFYVAE

(19) United States
(12) Patent Application Publication
(10) Pub. No.: US 2012/0021479 A1
(43) Pub. Date: Jan. 26, 2012

(54) Systems of Hydrogen Production in Bacteria

(75) Inventors: Pamela Silver, Cambridge, MA (US); David Savage, Cambridge, MA (US); Christina Agapakis, Brookline, MA (US)

(73) Assignee: PRESIDENT AND FELLOWS OF HARVARD COLLEGE, Cambridge, MA (US)

(21) Appl. No.: 12/672,037

(22) PCT Filed: Aug. 4, 2008
(86) PCT No.: PCT/US2008/009375

§ 371 Date: Jun. 28, 2011

Related U.S. Application Data
(60) Provisional application No. 60/963,472, filed on Aug. 3, 2007.

(51) Int. Cl. C12P 3/00; C12N 1/13; C12N 1/21

(52) U.S. Cl. 435/168; 435/252.3; 435/257.2; 435/252.33

(57) ABSTRACT

This invention relates to engineered bacterial systems such as engineered cyanobacterial systems and to methods of using these bacterial systems to generate hydrogen.

### RELATED APPLICATIONS

This patent application claims the benefit of U.S. Provisional Patent Application Ser. No. 60/963,472, filed Aug. 3, 2007, the contents of which are herein incorporated by reference in their entirety.

### FIELD OF THE INVENTION

This invention relates to engineered bacterial systems such as engineered cyanobacterial systems and to methods of using these bacterial systems to generate hydrogen.

### BACKGROUND OF THE INVENTION

The most common industrial methods for producing hydrogen include steam reformation of natural gas, coal gasification, and splitting water with electricity typically generated from fossil fuels. These energy-intensive industrial processes release carbon dioxide and other greenhouse gases and pollutants as by-products.

Accordingly, there currently exists a need for cost-effective compositions, systems and methods of increasing production of hydrogen without negative side effects, such as pollution.

### SUMMARY OF THE INVENTION

This invention provides engineered bacterial systems such as engineered cyanobacterial systems and methods of using these bacterial systems to generate hydrogen. The invention provides isolated bacterial cells that include a nucleic acid encoding a fusion protein comprising a subunit of photosystem I (PSI) coupled to a heterologous hydrogenase. The PSI subunit is, for example, a PsaE subunit. The PSI subunit is coupled directly or indirectly to the hydrogenase. For example, the PSI subunit, and the hydrogenase are indirectly coupled using a linker moiety. A linker is placed between the PSI subunit and the hydrogenase. The length of the linker is varied wherein lengthening of the linker region progressively leads to a reduction in the rate of interaction between the PSI subunit and the hydrogenase. Linker region lengths range from 2 amino acids or about 8 angstroms to 50 amino acids or about 200 angstroms. A preferred linker length is about 25 to 40 angstroms, and a more preferred linker length is about 35 Angstroms.

The bacterial cells are, for example, cyanobacterial cells. Suitable bacterial cells for use in the compositions, systems and methods provided herein include, for example, a bacterial cell selected from a Synechococcus elongatus cell, a Synechocystis cell, a Thermosynechococcus elongatus cell, an E. coli cell, a wild cyanobacteria cell, and a Prochloroccus cell. For example, the bacterial cell is a Synechococcus elongatus PCC7942 cell.

The heterologous hydrogenase is, for example, an O₂ tolerant hydrogenase. In some embodiments, the O₂ tolerant hydrogenase is an O₂ tolerant [NiFe] hydrogenase. For example, the heterologous hydrogenase is a hoxK subunit of membrane bound hydrogenase (MBH). The hoxK subunit of MBH is, for example, derived from Ralstonia eutropha. In some embodiments, the heterologous hydrogenase is an [FeFe] hydrogenase. For example, the heterologous hydrogenase is an [FeFe] hydrogenase derived from a Chlamydomonas species, a Clostridium species or a Ralstonia species. In some embodiments, the [FeFe] hydrogenase includes one or more mutations relative to the most closely related natural hydrogenase, wherein the mutation confers enhanced enzymatic activity in the presence of oxygen. The most closely related natural hydrogenase is identified, for example, by performing a BLAST search using the NCBI BLAST server.

In some embodiments, the [FeFe] hydrogenase includes an amino acid alteration relative to the most closely related natural hydrogenase, wherein the alteration places an amino acid with a higher molecular weight than the amino acid residue in the corresponding position in the most closely related natural hydrogenase. The table below provides the molecular weight of each amino acid residue. Those of ordinary skill in the art will readily appreciate which amino acid alterations place an amino acid with a higher molecular weight than the amino acid residue in the corresponding position in the most closely related natural hydrogenase.

| Amino acid | Molecular weight (g/mol) |
|---|---|
| Isoleucine | 131.1736 |
| Leucine | 131.1736 |
| Lysine | 146.1882 |
| Methionine | 149.2124 |
| Phenylalanine | 165.1900 |
| Threonine | 119.1197 |
| Tryptophan | 204.2262 |
| Valine | 117.1469 |
| Arginine | 174.2017 |
| Histidine | 155.1552 |
| Alanine | 89.0935 |
| Asparagine | 132.1184 |
| Aspartate | 133.1032 |
| Cysteine | 121.1590 |
| Glutamate | 147.1299 |
| Glutamine | 146.1451 |
| Glycine | 75.0669 |
| Proline | 115.1310 |
| Serine | 105.0930 |
| Tyrosine | 181.1894 |

In some embodiments, the [FeFe] hydrogenase includes an amino acid alteration relative to the most closely related natural hydrogenase, wherein the alteration places an amino acid with a higher molecular weight than leucine at a position selected from the group 136, 163, 384, 464, and 469 numbered according the sequence of the [FeFe] hydrogenase from Chlamydomonas reinhardtii, wherein the most closely related natural hydrogenase has an amino acid with a molecular weight equal to or less than that of leucine at the corresponding position. In some embodiments, the [FeFe] hydrogenase includes an amino acid alteration relative to the most closely related natural hydrogenase, wherein the alteration places an amino acid with a higher molecular weight at a position selected from the group 275, 284, 431, 435, 462, 468, and 493 numbered according the sequence of the [FeFe] hydrogenase from Clostridium pasteurianum, wherein the most closely related natural hydrogenase has an amino acid with a molecular weight equal to or less than that of substituted amino acid at the corresponding position.

The PSI subunit is, for example, a PsaE subunit derived from a cyanobacterial PSI. In some embodiments, the PSI subunit is coupled to the heterologous hydrogenase via a linker. For example, the PSI subunit is linked to the c-terminus of the heterologous hydrogenase. In some embodiments, the heterologous hydrogenase is a hoxK subunit of MBH. The linker is, for example, an amino acid sequence.

The bacterial cells also include, in some instances, a promoter, such as, for example, a photosynthesis-related promoter. In one embodiments, the promoter is psaAB. The bacterial cells also include, in some embodiments, a nucleic acid encoding a maturation factor.

The invention also provides systems for producing biological hydrogen in which the system includes any of the bacterial cells described herein. The invention also provides methods for producing hydrogen by providing a light source; and using the isolated bacterial cells described herein, for example, the isolated cyanobacterial cells, to drive the reaction:

H₂O+photons→½+H₂.

This invention provides biological compositions, systems and methods for producing hydrogen using an engineered bacterial system. For example, the invention provides biological compositions, systems and methods for producing hydrogen from engineered photosynthetic machinery in cyanobacteria. The biological compositions, systems and methods are used to produce hydrogen gas, a renewable form of energy, from sunlight. The hydrogen produced is used in a variety of applications, including, for example, fuel cells. Fuel cells use hydrogen and oxygen to create electricity and effectively produce zero or near-zero emissions, with only water and heat as byproducts. They can be used in various applications, from portable devices to buildings to vehicles.

The biological machinery of photosynthesis has been rewired to catalyze the conversion of sunlight into hydrogen gas, a high energy compound with innumerable uses. Prior to the instant invention, has not been demonstrated in vivo due to many technical reasons. The methods and systems provided herein express a functional oxygen-insensitive hydrogenase, the enzyme which catalyzes hydrogen production, in a photosynthetic bacterium. This hydrogenase is then directly linked to photosynthesis through a genetic fusion, and electrons generated by light-capture are directly used to produce hydrogen gas.

The genetically transformable cyanobacterium Synechococcus elongatus PCC 7942 is a model photosynthetic organism. The x-ray structure of photosystem I (PSI) from a closely related species is known, facilitating the engineering of the complex. This strain lacks an endogenous hydrogenase.

To create a photosynthetic organism that efficiently produces hydrogen via photosynthesis, a genetic fusion between the membrane-bound hydrogenase from Ralstonia and PsaE of Synechococcus has been constructed. This construct is expressed from the photosystem I promoter of Synechococcus and transformed into a psaE mutant strain. Also, linkers of three to ten amino acids are optionally used to optimize electron transfer, and the protein is histidine-tagged to allow for easy purification and detection. Concurrently, the membrane-bound maturation operon is integrated and expressed under the control of a constitutive promoter.

While the examples provided herein use PsaE, other photosystem genes are useful in the genetic fusions provided herein. For example, psaC or psaD are useful in the genetic fusions provided herein.

The compositions of the invention include a fusion protein or polypeptide, also referred to herein as a non-natural protein or polypeptide, that includes a hydrogenase moiety and a ferredoxin moiety. In some embodiments, the hydrogenase moiety and the ferredoxin moiety are linked, directly or indirectly, using a linker. The linker is any suitable coupling mechanism, including, for example, a glycine- and serine-rich amino acid linker such as (Gly₄Ser)ₙ, where n is an integer from 1 to about 10, a linker consisting of glycine, serine, alanine, and threonine, and other linkers that have been described in the art of protein engineering. In some embodiments, the hydrogenase moiety and the ferredoxin moiety are derived from different organisms. The hydrogenase moiety is, for example, an [FeFe] hydrogenase or an [NiFe] hydrogenase. The hydrogenase moiety is derived from species such as, for example, a Chlamydomonas species, Clostridium species, or a Ralstonia species.

In embodiments where the hydrogenase moiety is derived from a Ralstonia species, the hydrogenase moiety is, for example, the Ralstonia eutropha membrane-bound hydrogenase in which the C-terminal membrane attachment segment has been removed. The Ralstonia membrane-bound hydrogenase, lacking the membrane attachment segment, is also used to construct fusions with a photosystem protein or polypeptide. The hydrogenase moiety and the photosystem protein are linked directly, or optionally, through a linker. The proteins are then expressed in photosynthetic cells in the presence of the maturation factors that are encoded in the Ralstonia operon that also encodes the membrane-bound hydrogenase.

The ferredoxin moiety is, for example, an Fe₂S₂ iron-sulfur cluster. In a preferred embodiment, the ferredoxin is preferably a chloroplast-derived ferredoxin, for example from spinach. Alternatively, the ferredoxin is from a photosynthetic bacterium.

In some embodiments, the fusion proteins provided herein also include a photosystem protein or polypeptide moiety. The photosystem protein or polypeptide moiety includes, for example the following photosystem proteins and termini within Photosystem I: the N- and C-termini of the proteins PsaC, PsaD, and PsaE are preferred junction sites. The N-terminus of PsaA and PsaB are used, as well as the C-terminus of PsaF and/or PsaI, the N-terminus of PsaL, the C-terminus of PsaM, and/or the N-terminus of PsaX. Fusions of this type have the effect of placing the ferredoxin and the hydrogenase on the same side of the thylakoid membrane as the iron-sulfur clusters of Photosystem I, such that electron transfer to the hydrogenase is enhanced.

In some embodiments, the hydrogenase moiety includes one or more mutations relative to the most closely related natural hydrogenase, such that the mutation confers enhanced enzymatic activity in the presence of oxygen.

The invention also provides the nucleic acids encoding the fusion proteins that include a hydrogenase moiety and a ferredoxin moiety. These nucleic acids are used in cells, for example, in photosynthetic cells. In a preferred embodiment, the photosynthetic cell is a cell in which the endogenous plant-type ferredoxin activity has been reduced or eliminated, for example by mutation. Suitable cells include, for example, cyanobacteria such as Synechococcus, Synechocystis, and Prochloroccus species, such as Synechococcus elongatus 7942 and Thermosynochococcus elongatus BP-1.

Also provided herein are proteins or polypeptides that include an [FeFe] hydrogenase moiety having an amino acid alteration relative to the most closely related natural hydrogenase. In some embodiments, the [FeFe] hydrogenase moiety has an amino acid alteration relative to the most closely related natural hydrogen, such that the alteration places an amino acid with a higher molecular weight than leucine at a position selected from the group 136, 163, 384, 464, and 469 numbered according the sequence of the [FeFe] hydrogenase from Chlamydomonas reinhardtii (SEQ ID NO: 11), wherein the most closely related natural hydrogenase has an amino acid with a molecular weight equal to or less than that of leucine at the corresponding position.

In some embodiments, the [FeFe] hydrogenase moiety has an amino acid alteration relative to the most closely related natural hydrogen, such that the alteration places an amino acid with a higher molecular weight at a position selected from the group 275, 284, 431, 435, 462, 468, and 493 numbered according to the sequence of the [FeFe] hydrogenase from Clostridium pasteurianum (SEQ ID NO: 12), wherein the most closely related natural hydrogenase has an amino acid with a molecular weight equal to or less than that of substituted amino acid at the corresponding position. In some embodiments, protein or polypeptide that includes the [FeFe] hydrogenase also includes a ferredoxin moiety.

In a preferred embodiment, the [FeFe] hydrogenase is at least 80%, 85%, 90%, 91%, 92%, 93%, 94%, 95%, 96%, 97%, 98%, or 99% identical to the sequence of the [FeFe] hydrogenase of Clostridium pasteurianum (SEQ ID NO: 12) or Clostridium acetobutylicum (SEQ ID NO: 20), but that has one or more of the following amino acids at the following positions (numbered according to the Clostridium pasteurianum sequence of SEQ ID NO: 12): Val275, Ala280, Leu284, Leu287, Tyr417, Ser427, Val431, Phe435, Gln435, Leu435, Leu461, Trp466, Phe468, or the combination of Lys or Arg at position 464 with Glu at position 288. In some embodiments, a protein or polypeptide that includes the [FeFe] hydrogenase also includes a ferredoxin moiety.

In some embodiments, the [FeFe] hydrogenase includes one or more of the following sets of amino acids when combined at the following positions (as numbered according to the Clostridium pasteurianum sequence of SEQ ID NO: 12): the combination of Val431 and Phe468; the combination of Leu435 and Leu284; the combination of Leu435 and Ile284; the combination of Leu435 and Leu287; the combination of Leu435 and Leu287 and Ile284; the combination of Leu435 and Leu287 and Leu284; the combination of Arg 464 and Glu 288 and Gly289; the combination of Val431 and Phe468; the combination of Leu435 and Leu284 and Tyr417; the combination of Leu435 and Ile284 and Val431; and the combination of Leu435 and Leu287 and Trp466. In some embodiments, protein or polypeptide that includes the [FeFe] hydrogenase also includes a ferredoxin moiety.

In some embodiments where the [FeFe] hydrogenase includes one or more of the amino acid combinations listed above, the [FeFe] hydrogenase also includes an amino acid alteration relative to the most closely related natural hydrogenase, such that the alteration places an amino acid with a higher molecular weight at a position selected from the group 275, 284, 462, 468, and 493 numbered according to the sequence of the [FeFe] hydrogenase from Clostridium pasteurianum (SEQ ID NO: 12), wherein the most closely related natural hydrogenase has an amino acid with a molecular weight equal to or less than that of substituted amino acid at the corresponding position. In some embodiments, a protein or polypeptide that includes the [FeFe] hydrogenase also includes a ferredoxin moiety.

The invention also provides the nucleic acids encoding the proteins or polypeptides that includes a [FeFe] hydrogenase moiety having an amino acid alteration as compared to the most closely related natural hydrogenase. These nucleic acids are used in cells, for example, in photosynthetic cells.

The term “isolated”, as in isolated nucleic acid molecule or isolated bacterial cell, as used herein, refers to a molecule or cell that is separated from other molecules and/or cells which are present in the natural source of the molecule or cell. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. Moreover, an “isolated” nucleic acid molecule is substantially free of other cellular material, or culture medium, or of chemical precursors or other chemicals.

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present specification will control.

Other features, objects, and advantages of the invention will be apparent from the description and drawings. All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same.

### BRIEF DESCRIPTION OF THE FIGURES

FIG. 1A-B. The hydrogenase active site of A.) the [NiFe]-hydrogenases and B.) the [FeFe]-hydrogenases (Vincent et al. 2005. Journal of the American Chemical Society 127, 18179-18189). X, Y, and L represent ligands whose presence is inferred from electron density in crystal structures, but which have not been chemically defined.

FIG. 2A-C. Genes involved in hydrogen production in Chlamiydomonas reinhardtii. A.) HydA1, the [FeFe]-hydrogenase. B.) The maturation factor HydEF and C.) The maturation factor HydG (Ghirardi et al. 2007. Annual Review of Plant Biology 58, 71-91).

FIG. 3. The proposed mechanism for hydrogenase protein maturation. HydEF and HydG form a complex that catalyzes the formation of the active site through a radical-SAM mechanism and insert it into the precursor hydrogenase protein with energy from GTP hydrolysis (Leach, M. R. and Zamble, D. B. 2007. Current Opinion in Chemical Biology 11, 159-165).

FIG. 4 Molecular dynamics simulations of the hydrogenase protein from Clostridium pasteurianum found two main channels through which gasses can travel from the surface of the protein to the active site. A.) Diffusion pathways for hydrogen. B.) Channels for oxygen (Cohen et al. 2005. Structure 13, 1321-1329).

FIG. 5. Predicted docking interaction between ferredoxin (lighter model at bottom) and hydrogenase (darker model at top). Homology model and docking structure is from Chang et. al. (2007) Biophysical J. 93, 3034.

FIG. 6 Gas chromatography trace of in vitro hydrogen production assay for hydrogenase-ferredoxin fusion protein. The larger the area of the peak, the more hydrogen is produced. E. coli alone produce very little hydrogen (smallest lowest curve). In this experiment, E. Coli expressing the Chlamydomonas hydrogenase and spinach ferredoxin as separate proteins (second-lowest curve) and E. Coli expressing only the Chlamydomonas hydrogenase (second-highest curve) produced about equal amounts of hydrogen, while E. Coli expressing the ferredoxin-hydrogenase fusion protein produced the largest amount of hydrogen (highest curve).

FIG. 7. Diagram of the Ralstonia eutropha hydrogenase fused to PSI of Thermosynechococcus elongatus. Electrons are elevated to a higher energy level by shining light on PSI. These electrons are then shuttled directly to the hydrogenase enzyme which uses them to produce molecular hydrogen.

FIG. 8A-F. Correlating amino acid size, channel volume, half-life, and oxygen concentration for [FeFe]-hydrogenases. Properties of several different [FeFe] hydrogenases from different organisms were obtained from the scientific literature, and then plotted as scatter graphs. A. The X axis represents the level of oxygen in the environment of each organism, where 100% indicates atmospheric levels of oxygen. The Y axis represents the half-life of the organism's hydrogenase in the presence of atmospheric oxygen. B. The X axis represents the half-life of the organism's hydrogenase in the presence of atmospheric oxygen. The Y axis represents the average size of amino acid side chain in the putative gas channels of the hydrogenases. C. The X axis represents the level of oxygen in the environment of each organism, where 100% indicates atmospheric levels of oxygen. The Y axis represents the average size of amino acid side chain in the putative gas channels of the hydrogenases. Note that the scale of the Y axis differs in FIGS. 8B and 8C. D. The X axis represents the average size of amino acid side chain in the putative gas channels of the hydrogenases. The Y axis represents the volume of the gas channels of the hydrogenases. E. The X axis represents the level of oxygen in the environment of each organism, where 100% indicates atmospheric levels of oxygen. The Y axis represents the volume of the gas channels of the hydrogenases. F. The X axis represents the half-life of the organism's hydrogenase in the presence of atmospheric oxygen. The Y axis represents the volume of the gas channels of the hydrogenases.

FIG. 9A-C. Results of a CASTp void search. A.) Gas channels from Chlamydomonas reinhardtii. B.) Gas channels from Clostridium pasteurianum. C.) Computationally mutated gas channels from Chlamydomonas reinhardtii. Mutations based on comparison with Clostridium pasteurianum structure. Spheres represent regions within the protein in which a void of at least 1.4 Angstroms was observed. The shading of the spheres indicates different subregions of the gas channels.

FIG. 10A-B. Mutations of the Chlamydomonas reinhardtii [FeFe]-hydrogenase. Aligned protein structures are shown with the homology model of the C. reinhardtii protein superimposed onto the Clostridium pasteurianum X-ray crystal model. Amino acids at positions 163 and 384 (FIG. 10A) and 136, 424 and 469 (FIG. 10B) have side chains that protrude into the gas channel (FIG. 9) and are smaller in C. reinhardtii than in C. pasteurianum. A.) Proposed gas channel A, indicating Leu163 and Leu384 as sites of useful mutation. B.) Proposed gas channel B, indicating Leu136, Leu464, and Leu469 as sites of useful mutation.

FIG. 11A-B. Results from NAMD molecular dynamics simulations of hydrogenases. The Y-axes show the volume in cubic Angstroms of gas channels from different hydrogenases is compared at different frames of the molecular dynamics simulation. A.) Hydrogenase from C. pasteurianum and C. reinhardtii. B.) Comparison of wild type and mutant C. reinhardtii with mutations designed to shrink gas channels.

FIG. 12A-B. Active site burial in hydrogenases. White arrow indicates active site. Clusters are iron-sulfur clusters involved in electron transfer. A.) Chlamydomonas reinhardtii hydrogenase (upper, darker model) and ferredoxin (lower, lighter model predicted docking structure (Chang, C. H. et al. 2007. Biophysics Journal, 93, 3034-3035). The active site is near the edge of the protein to facilitate interaction with ferredoxin. B.) The Clostridium pasteurianum hydrogenase has its active site buried deep within the protein interior, electrically connected via a series of iron-sulfur clusters.

FIG. 13. Gas chromatography traces from an in vitro hydrogen production assay. The area under each curve represents an amount of hydrogen produced. All samples are of E. Coli expressing the Chlamydomonas hydrogenase maturation factors unless otherwise indicated. Samples ranked in order of smallest to largest areas under the curve are: E. Coli BL21 without any [FeFe] hydrogenase gene; E. Coli expressing the Chlamydomonas hydrogenase and spinach ferredoxin; E. Coli expressing the Chlamydomonas hydrogenase (essentially identical to the previous sample); E. Coli expressing the Chlamydomonas hydrogenase fused to spinach ferredoxin; E. Coli expressing the Clostridium acetobutylicum hydrogenase and C. acetobutylicum maturation factors; and E. Coli expressing the Clostridium acetobutylicum hydrogenase and Chlamydomonas maturation factors (essentially identical to previous sample).

FIG. 14. The most variable and most invariant residues in the hydrogenase gas channels. This information can be used for structure-function mutagenesis analysis of the hydrogenase. The white arrow indicates the location of the active site.

FIG. 15. Schematic of the family shuffling technique.

FIG. 16. CLUSTALW analysis of three known iron-only hydrogenases and five sequences from the Sargasso Sea Database (Venter). Numbering of sequences is by the author. Cysteines that coordinate the N-terminal Fe clusters are boxed, catalytic H clusters are in bold with the Fe coordinating cysteines in highlight, proposed gas channel regions are underlined.

FIG. 17A-B and 1-5. Homology-based models of [FeFe]-hydrogenase sequences in the Sargasso Sea Database. Numbering (1-5) corresponds to SSDB-# from FIG. 16. Panel A. Clostridium pasteurianum hydrogenase 1.6 Å X-ray structure (Peters). Panel B. Homology-based model of Chlamydomonas reinhardtii hydrogenase HydA1.

FIG. 18. A schematic depiction of various engineered hydrogenase-linker-ferredoxin fusion proteins (FLH/HLF proteins). A. A fusion of a ferredoxin and a hydrogenase containing a single iron-sulfur cluster, such as a Chlamydomonas hydrogenase. 1. An irregular figure representing the hydrogenase moiety. 2. An oval representing the ferredoxin moiety. 3. A peptide linker that connects the hydrogenase to the ferredoxin. 4. A pair of black dots representing the two metal atoms at the hydrogenase active site. 5. A cube representing an Fe₄S₄ iron-sulfur cluster within the hydrogenase near the dimetal active site. 6. A diagonal representing an Fe₂S₂ iron-sulfur cluster within the ferredoxin moiety. B. A fusion of a ferredoxin and a hydrogenase containing four Fe₄S₄ iron-sulfur clusters and a single Fe₂S₂ iron-sulfur cluster, such as a Clostridium hydrogenase. 7. An irregular figure representing the hydrogenase moiety. 8. An oval representing the ferredoxin moiety. 9. A peptide linker that connects the hydrogenase to the ferredoxin. 10. A pair of black dots representing the two metal atoms at the hydrogenase active site. 11. A set of cubes representing the Fe₄S₄ iron-sulfur clusters and a diagonal representing the Fe₂S₂ cluster within the hydrogenase. 12. A diagonal representing an Fe₂S₂ iron-sulfur cluster within the ferredoxin moiety. C. A fusion of a ferredoxin and a [NiFe] hydrogenase containing three Fe₄S₄ iron-sulfur clusters, such as a Ralstonia or Desulfovibrio hydrogenase. 13. A partial egg-shaped figure representing the large subunit of the hydrogenase moiety. 14. A partial egg-shaped figure representing the small subunit of the hydrogenase moiety. 15. An oval representing the ferredoxin moiety. 16. A peptide linker that connects the hydrogenase to the ferredoxin. 17. A pair of black dots representing the two metal atoms at the hydrogenase active site. 18. A set of cubes representing the Fe₄S₄ iron-sulfur clusters within the large and small subunits of the hydrogenase. 19. A diagonal representing an Fe₂S₂ iron-sulfur cluster within the ferredoxin moiety.

FIG. 19. A schematic depiction of an engineered hydrogenase-linker-ferredoxin-linker-Photosystem I protein complex (the “HLFLP” configuration). 1. An irregular figure representing the hydrogenase moiety. 2. An oval representing the ferredoxin moiety. 3. A rectangle representing the transmembrane segments of the Photosystem I moiety. 4. A diagonally striped peak representing the PsaE moiety. 5. A peptide linker that connects the hydrogenase to the ferredoxin. 6. A peptide linker that connects the ferredoxin to the PsaE moiety. 7. A checkerboard pattern representing the thylakoid membrane in which the Photosystem I is embedded. 8. A pair of black dots representing the two metal atoms at the hydrogenase active site. 9. A cube representing an Fe₄S₄ iron-sulfur cluster within the hydrogenase near the dimetal active site. 10. A diagonal representing an Fe₂S₂ iron-sulfur cluster within the ferredoxin moiety. 11. A pair of diagonal lines representing the ‘special pair’ of chlorophyll molecules at the center of Photosystem I. 12. The three Fe₄S₄ iron-sulfur clusters within Photosystem I.

FIG. 20. Schematic illustration of the function of an HLFLPase. A. A photon impinges on Photosystem I and its energy is transferred, directly or indirectly, to the ‘special pair’ of chlorophylls. The net result is that an electron is excited and tunnels into the iron-sulfur clusters in Photosystem I. B. The ferredoxin that is tethered to Photosystem I by a linker preferentially interacts with the Photosystem and receives the excited electron from the iron-sulfur cluster in PsaD. C. The tethered ferredoxin, now reduced, dissociates from Photosystem I and preferentially donates its electron to the hydrogenase to which it is tethered by a linker.

FIG. 21. Schematic diagram of the lac_MBHpatent expression vector.

FIG. 22. Alignment of [NiFe] hydrogenase small subunits.

FIG. 23. The roles of Photosystem II (PSII) and Photosystem I (PSI) in photosynthesis.

FIG. 24. Structure of Photosystem I (PSI).

FIG. 25. Schematic representation of electrons in Photosystem I (PSI).

FIG. 26. Schematic representation of electron excitation in Photosystem I (PSI).

FIG. 27. Schematic representation of electron excitation in Photosystem I (PSI) and design concept of linking proteins to channel electrons to Hydrogenase via Ferredoxin.

FIG. 28. Schematic representation of how constrained protein movement channels electrons into Hydrogenase from Photosystem I via Ferredoxin to produce molecular hydrogen.

FIG. 29. A representative plasmid encoding a maturation factor used for making an E. Coli BL21 DE3 strain for expression of an FeFe hydrogenase. The plasmid is a modified pACYCDuet-1.

FIG. 30. Schematic representation depicting the process of photosynthesis.

FIG. 31. Schematic representation of membrane-bound hydrogenase (MBH).

FIGS. 32A and 32B. Photosystem I (PSI) (Panel A) and MBH fused to PsaE bound to PSI (Panel B). e⁻ pathway is denoted with arrows.

FIG. 33. Schematic representation of hydrogenase genomic integration.

FIG. 34. Amino acid sequences of various hydrogenases.

FIGS. 35A-35E. Schematic representations of various plasmids used for engineering bacteria to express hydrogenases.

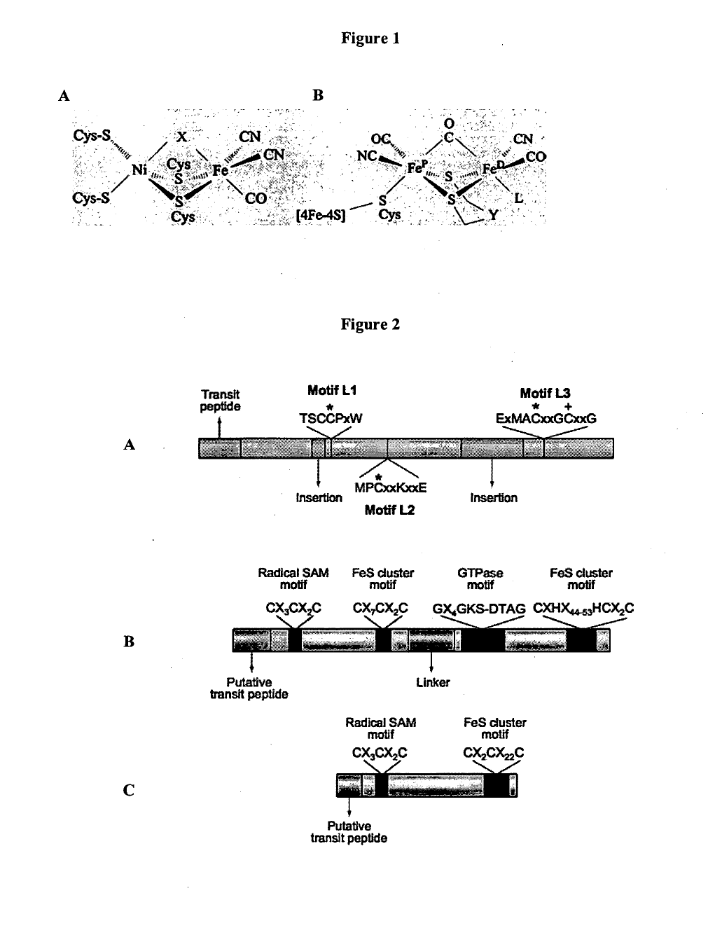

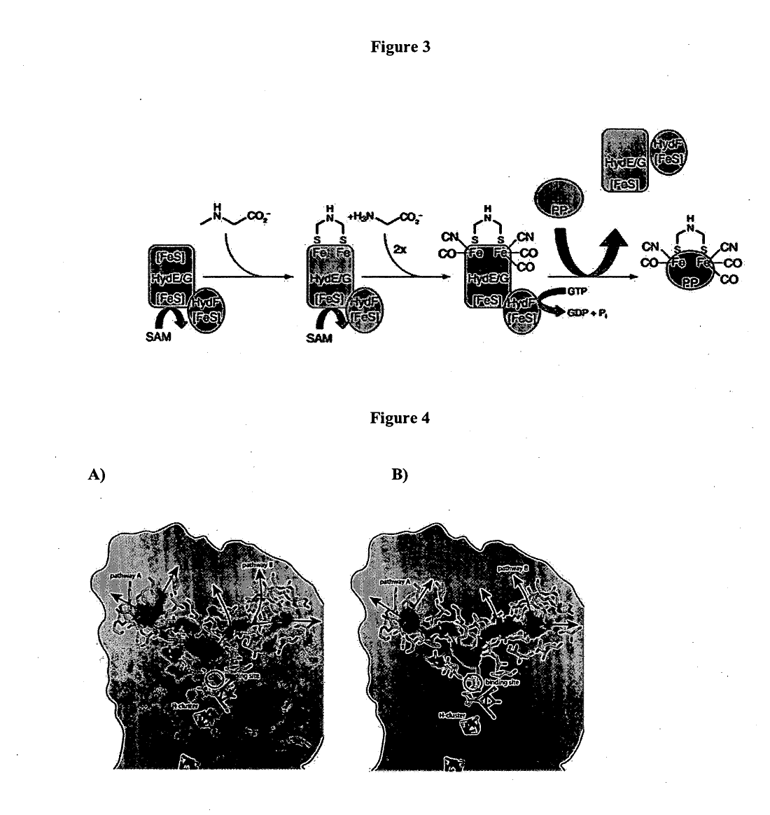

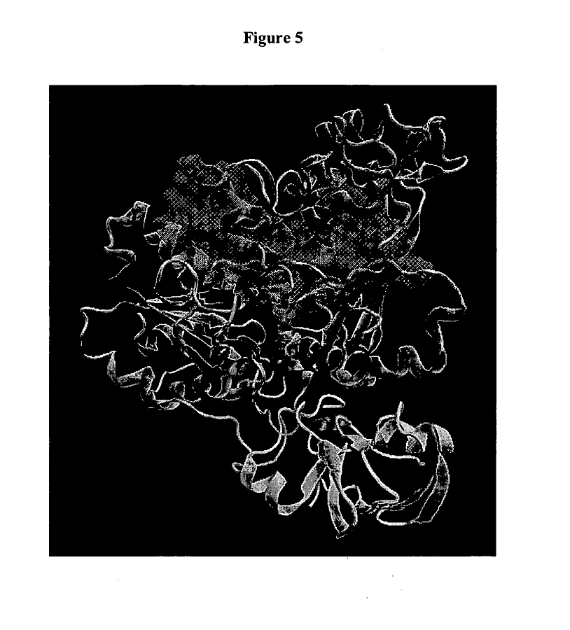

### DETAILED DESCRIPTION OF THE INVENTION

The invention provides a solar-based energy economy as a solution to the problems of sustainability and rising atmospheric CO₂ levels. In particular, the invention provides engineered biological systems that convert solar radiation into convenient forms of chemical energy such as H₂. The biological systems and methods provided herein use hydrogenases, enzymes which catalyze the reaction:

2H⁺+2e⁻ [symbol] H₂

Previous attempts to use hydrogenases in engineered systems have been hampered by poor understanding of their maturation in vivo. The biological systems and methods provided herein express functional hydrogenases in non-native organisms. These hydrogenases are expressed in bacteria, preferably cyanobacteria, to make a genetic link to photosynthesis, thereby creating a novel photosystem complex capable of catalyzing photons into hydrogen gas.

The hydrogen produced is used in a variety of applications, including, for example, fuel cells. Fuel cells use hydrogen and oxygen to create electricity and effectively produce zero or near-zero emissions, with only water and heat as byproducts. They can be used in various applications, from portable devices to buildings to vehicles.

The methods provided herein use engineered bacterial cells to efficiently generate hydrogen. In principle, a biological system should be able to catalyze photosynthesis, i.e., the following reaction:

6CO₂+12H₂O+photons→C₆H₁₂O₆+6O₂+6H₂O

Photosynthesis may simply be defined as the conversion of light energy into chemical energy by living organisms. It is affected by its surroundings and the rate of photosynthesis is affected by the concentration of carbon dioxide, the intensity of light, and the temperature.

Photosynthesis occurs in two stages. In the first phase light-dependent reactions or photosynthetic reactions (also called the light reactions) capture the energy of light and use it to make high-energy molecules. During the second phase, the light-independent reactions (also called the Calvin-Benson Cycle, and formerly known as the Dark Reactions) use the high-energy molecules to capture carbon dioxide (CO₂) and make the precursors of carbohydrates.

In the light reactions one molecule of the pigment chlorophyll absorbs one photon and loses one electron. This electron is passed to a modified form of chlorophyll called pheophytin, which passes the electron to a quinone molecule, allowing the start of a flow of electrons down an electron transport chain that leads to the ultimate reduction of NADP into NADPH. In addition, it serves to create a proton gradient across the chloroplast membrane; its dissipation is used by ATP Synthase for the concomitant synthesis of ATP. The chlorophyll molecule regains the lost electron by taking one from a water molecule through a process called photolysis, that releases oxygen gas as a waste product.

In the light-independent or dark reactions the enzyme RuBisCO captures CO₂ from the atmosphere and in a process that requires the newly formed NADPH, called the Calvin-Benson cycle releases three-carbon sugars which are later combined to form sucrose and starch.

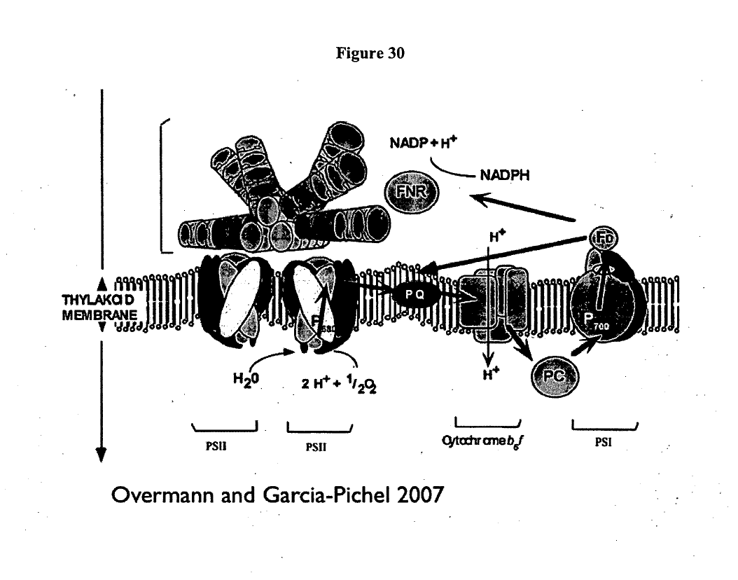

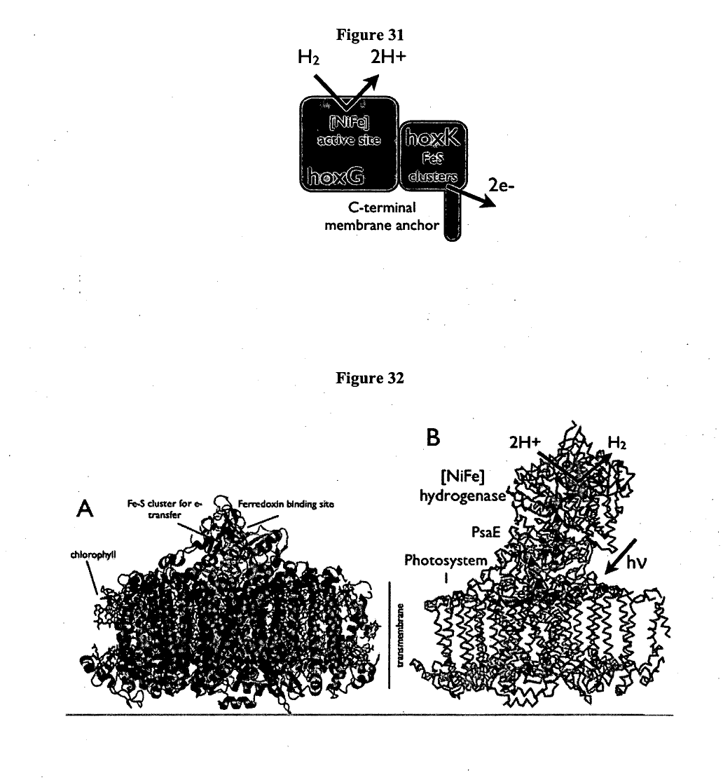

Photosynthesis is the entry for nearly all high energy electrons into biogeochemical cycles. Photosynthesis (FIG. 30) is an electron transfer pathway in which five key events occur:
i. water is split and an electron transferred to photosystem II;
ii. light absorbed by photosystem II is used to excite this electron to a high energy state;
iii. the electron is transferred to photosystem I (PSI) and a proton gradient is generated;
iv. photosystem I uses absorbed light to excite the electron to an even higher state; and
v. the electron is transferred to ferredoxin to be used in carbon fixation.

The biological systems and methods provided herein were designed using the principle that these electrons can be used as products in novel chemical redox reactions, such as reducing two protons to molecular hydrogen, by rewiring, preferably through rational design, this electrical pathway.

To accomplish this rewiring, the biological systems and methods provided herein use hydrogenases. Hydrogenases are enzymes which catalyze the reaction

2H⁺+2e⁻ [symbol] H₂.

This reaction is reversible, and there are hydrogenases existent in nature that catalyze both the forward and reverse reaction. Many naturally occurring cyanobacteria express hydrogenases and produce a burst of hydrogen during the onset of photosynthesis. In cyanobacteria, as in most of nature, these enzymes are oxygen sensitive and turn off as oxygen accumulates from photosynthesis. This phenomenon acts as an electron “safety valve” to maintain cellular redox state and illustrates photosynthesis can in principle be linked to H₂ production.

Nearly all hydrogenases are oxygen sensitive. The knall-gas bacterium Ralstonia eutropha, however, harbors two unique hydrogenases, which are used to oxidize molecular hydrogen in the presence of oxygen. (Burgdorf et al., J. Mol. Microbiol. Biotech., vol. 10(2-4): 186-91 (2005), the contents of which are hereby incorporated by reference in their entirety). Both are “uptake” hydrogenases and transfer electrons from H₂ to a redox partner of less reducing potential via a unique nickel-iron active site and several iron-sulfur (FeS) clusters. Maturation of the functional enzyme involves a series of enzymatic reactions and requires up to 14 additional genes. Prior to the invention, poor understanding of hydrogenase maturation has held back their use in heterologous systems.

A 22 kb fragment of the hox operon is sufficient for maturation of the Ralstonia membrane bound hydrogenase (MBH). (Lenz, et al., J. Bacteriol., vol. 187(18): 6590-95 (2005). MBH is composed of two subunits. The gene hoxG encodes the catalytic subunit while hoxK is involved in membrane anchoring and electron transfer. Electrons are transferred using a network of iron-sulfur (FeS) clusters. This occurs via quantum tunneling and is highly dependent on distance and the relative electronic potentials between adjacent clusters. Thus, electrons are more likely to flow downhill from more negative potential to high potential. MBH consumes H₂ and the electrons are transferred to a membrane anchored cytochrome to be used in metabolism. In principle, the directionality of MBH could be reversed if electrons were transferred to hoxK with a potential more negative than that of the H₂ potential, −420 mV at cellular conditions. A candidate donor would be the Fb (−440 mV) FeS cluster of PSI. In this construction, event v) of photosynthesis, as described above, is skipped and electrons are directly shuttled away from ferredoxin into the production of H₂. Thus one could link photon capture to hydrogen production.

Prior to the understanding of hydrogenase maturation above, Ihara et al. (Photochem. and Photobiol., vol. 82(3): 676-82 (2006), the contents of which are hereby incorporated by reference in their entirety) demonstrated this linkage by constructing a genetic fusion based on atomic resolution structural models of the PsaE subunit of Photosystem I (PSI) (FIGS. 32A and 32B) and the hoxK subunit of the oxygen-insensitive, membrane-bound hydrogenase (MBH). Specifically, the membrane anchor of hoxK was replaced with a short linker (Ser-Gly-Gly) and PsaE. The fusion protein was expressed in Ralstonia (in the presence of endogenous maturation factors), purified, and reconstituted in vitro with PSI purified from a psaE-deficient cyanobacterium. In this construction, electrons are transferred directly from photosystem Ito the hydrogenase via tunneling between adjacent Fe—S clusters (FIG. 32). The reconstituted complex of Ihara produced hydrogen in a light-dependent manner but is limited by two shortcomings. First, the in vitro nature limits any broad applicability. Second, no attempt at optimizing the linker sequence between PsaE and hoxK was made. This lead to lower than expected H₂ production rates and significant competitive inhibition by ferredoxin, the native electron acceptor.

The biological systems and methods provided herein optimize that linkage in a living cell. In a preferred embodiment, cyanobacteria, such as Synechococcus, are used as the platform for expressing the hydrogenase and linking this expression to photosynthesis. Many naturally occurring cyanobacteria encode hydrogenases and produce a burst of hydrogen during the onset of photosynthesis. Without intending to be bound by theory, it is thought that production of hydrogen is turned off as oxygen accumulates from the Photosystem II reaction. This phenomenon may be akin to an electron “safety valve” to maintain cellular redox state and illustrates that photosynthesis is linked to H₂ production.

Cyanobacteria (commonly called blue-green algae) are oxygenic photolithoautotrophs that use nearly the same photosynthetic process as plants, but are amenable to the tools of molecular biology developed for yeast and bacteria.

Cyanobacteria have an elaborate and highly organized system of internal membranes which function in photosynthesis. Photosynthesis in cyanobacteria generally uses water as an electron donor and produces oxygen as a by-product, though some may also use hydrogen sulfide as occurs among other photosynthetic bacteria. Carbon dioxide is reduced to form carbohydrates via the Calvin cycle. In most forms the photosynthetic machinery is embedded into folds of the cell membrane, called thylakoids.

Cyanobacteria are the only group of organisms that are able to reduce nitrogen and carbon in aerobic conditions. The water-oxidizing photosynthesis is accomplished by coupling the activity of photosystem (PS) II and I. In anaerobic conditions, they are also able to use only PS I—cyclic photophosphorylation—with electron donors other than water (hydrogen sulfide, thiosulphate, or even molecular hydrogen). Furthermore, they share an archaebacterial property, which is the ability to reduce elemental sulfur by anaerobic respiration in the dark.

Synechococcus elongatus PCC7942 (hereafter Synechococcus), for example, is naturally transformable and grows to high cell density, making it a convenient “chassis” for synthetic biology techniques. The biological systems and methods provided herein use cyanobacteria, and preferably Synechococcus (or a closely related cyanobacteria), as a platform for genetic engineering.

Some photosynthetic bacteria naturally express hydrogenases. These hydrogenases sometimes produce a burst of hydrogen when initially exposed to light, but hydrogen production ceases when a sufficient amount of oxygen has accumulated as a result of photosynthesis. The production of hydrogen occurs by generating NADPH, which is then used to make hydrogen by the reaction NADPH+H⁺→H₂+NADP⁺. In the methods for producing hydrogen provided herein, the hydrogenase and hydrogen production is electronically coupled to a Photosystem (generally Photosystem I), rather than being chemically coupled to a photosystem through a small-molecule intermediate (such as NADPH). Thus, these methods involve the transfer of electrons from a photosystem to a hydrogenase by quantum-mechanical tunneling between iron-sulfur clusters. These iron-sulfur clusters lie in Photosystem I, and the hydrogenase, and optionally, in other iron-sulfur cluster proteins such as ferredoxin.

### EXAMPLES

### Example 1

### Expression of Hydrogenases Leading to Production of Hydrogen Gas in Bacteria

The biological systems and methods provided herein use cyanobacteria, and preferably Synechococcus elongatus PCC7942 (or a closely related cyanobacteria), as a platform for genetic engineering.

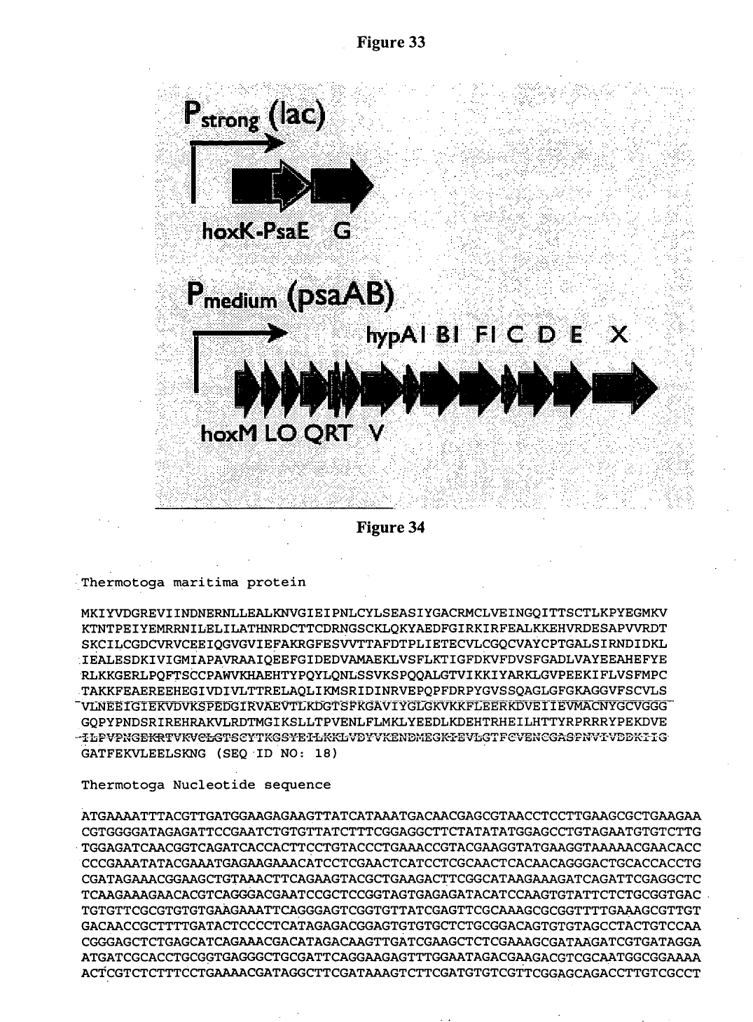

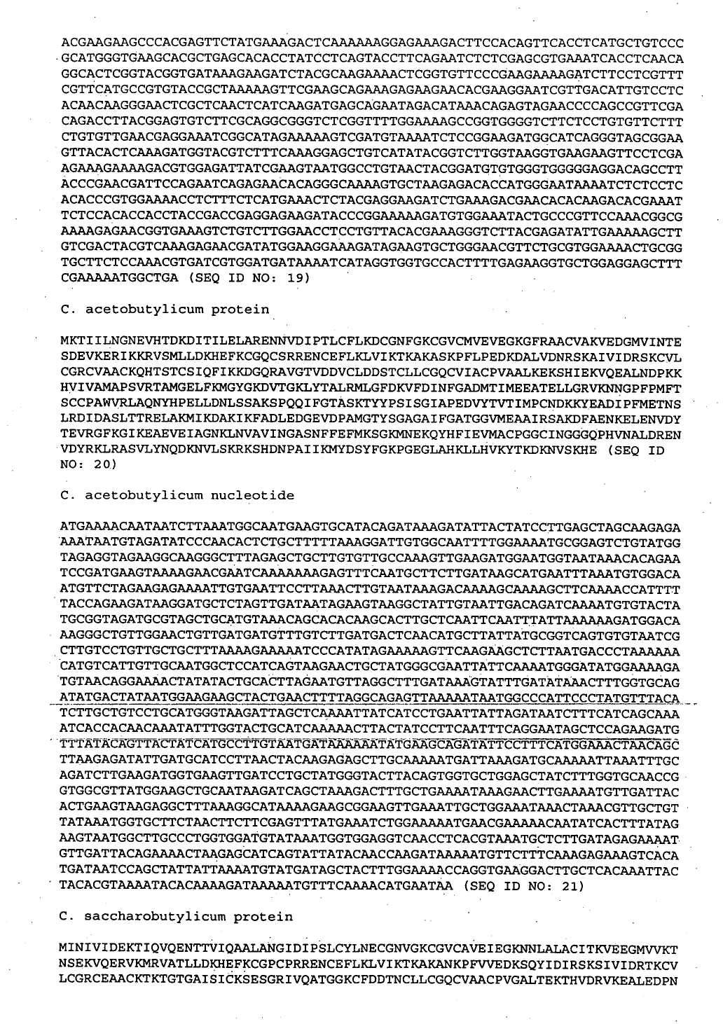

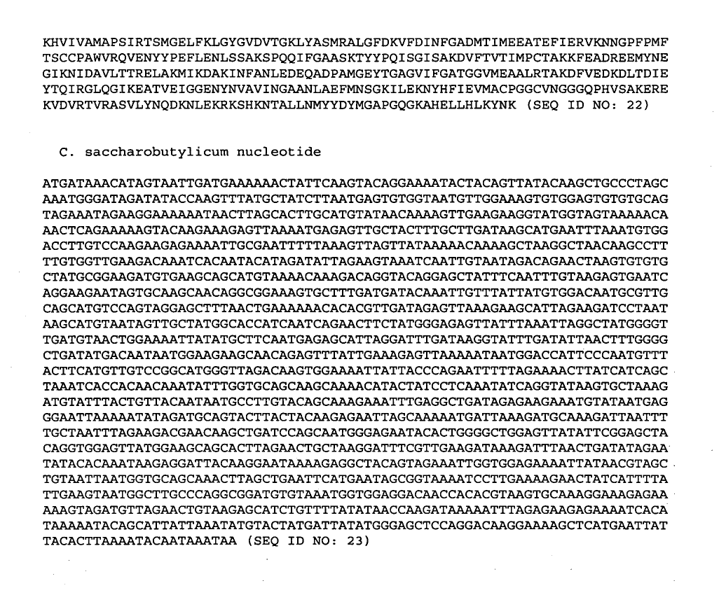

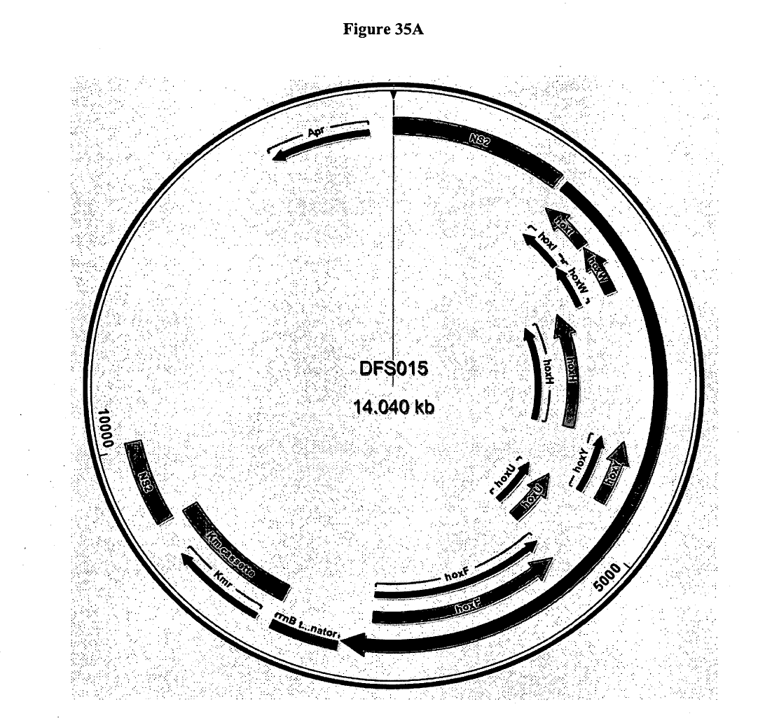

The basic expression strategy is shown in FIG. 33. PsaE is cloned from Synechococcus and fused to the c-terminus of hoxK. The initial fusion is made using a Ser-Gly-Ser linker, but following successful demonstration of function, a larger screen is used to identify the optimal fusion. The cloned hydrogenase structural genes with fusion to PsaE are integrated into the Synechococcus genome and placed under the control of a strong promoter such as the lac promoter from E. coli. (See Liu et al., J. Bacteriol., vol. 177(8): 2080-86 (1995), the contents of which are hereby incorporated in their entirety). The maturation factors are catalytic and needed at lower concentrations. The maturation factors are shown in FIG. 33. They are integrated into the genome under a medium strength promoter such as psaAB or similar photosynthesis-related promoter. Other photosynthesis-related promoters include, for example, psbAI, psbAII, psbDI, psaAB and lac (from E. coli). Synechococcus has a robust cirdian rhythm, and if necessary, hydrogenase expression and maturation is optimized to coincide with the optimum expression and activity of PSI. If necessary, PsaE is knocked out of the host.

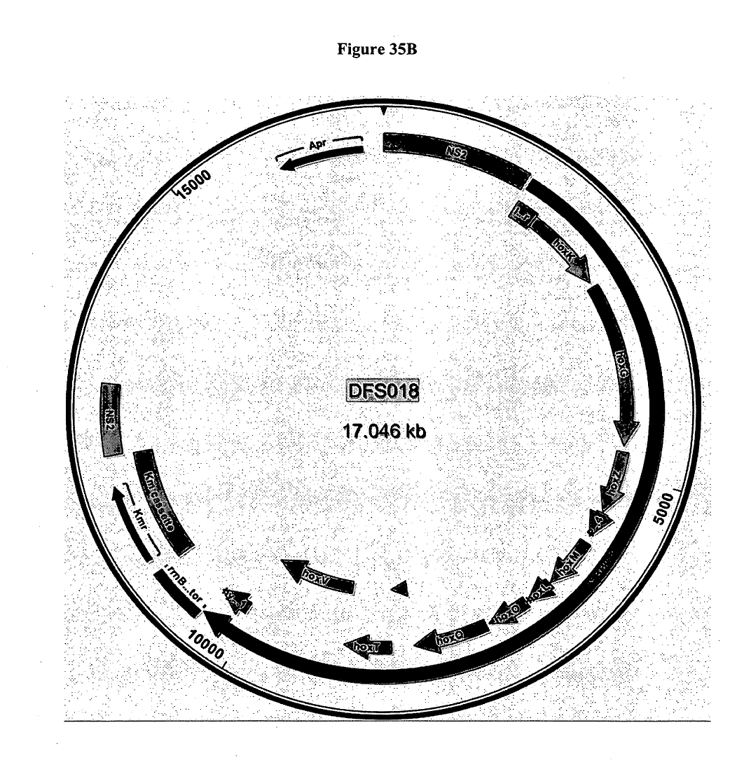

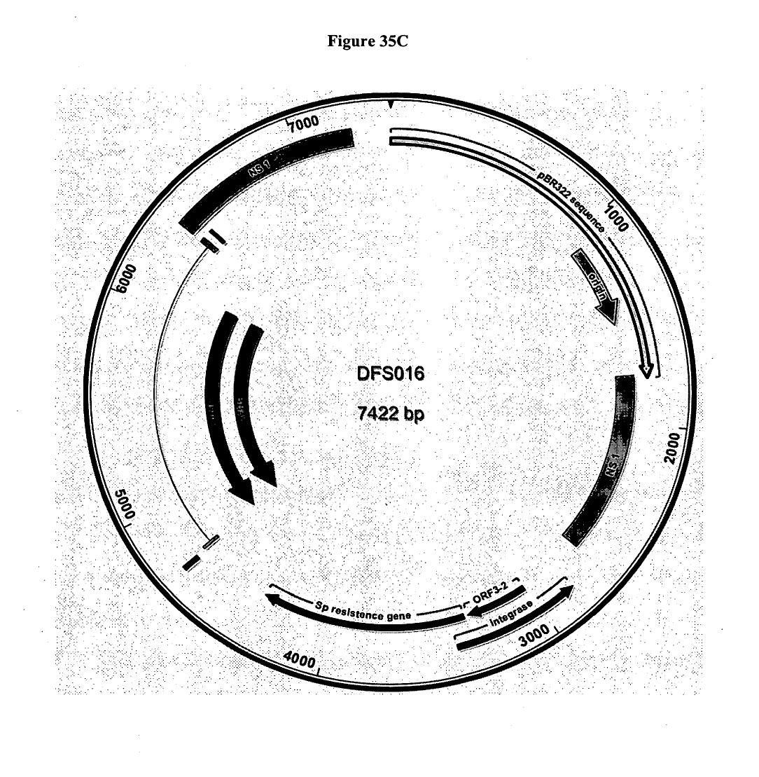

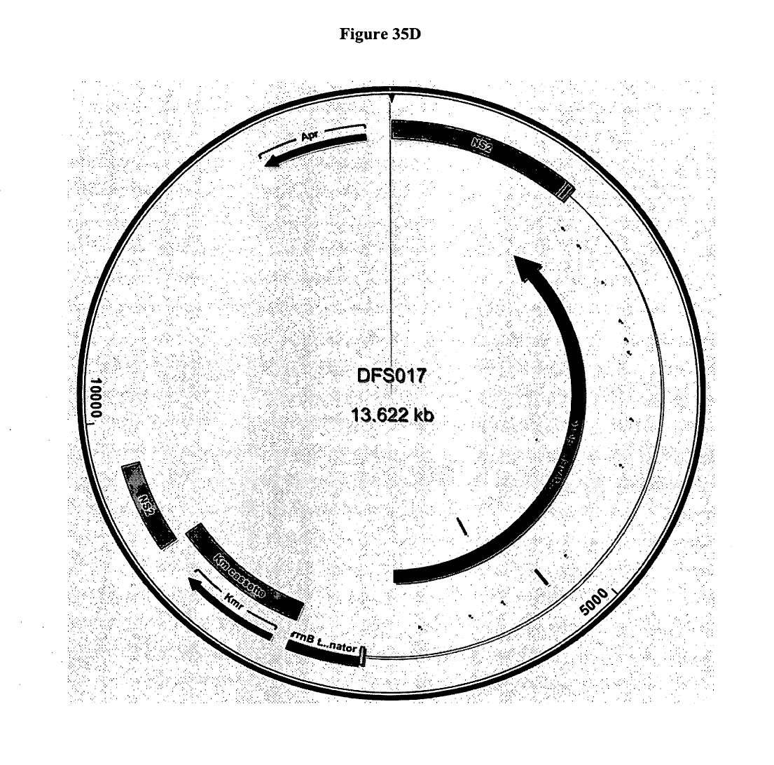

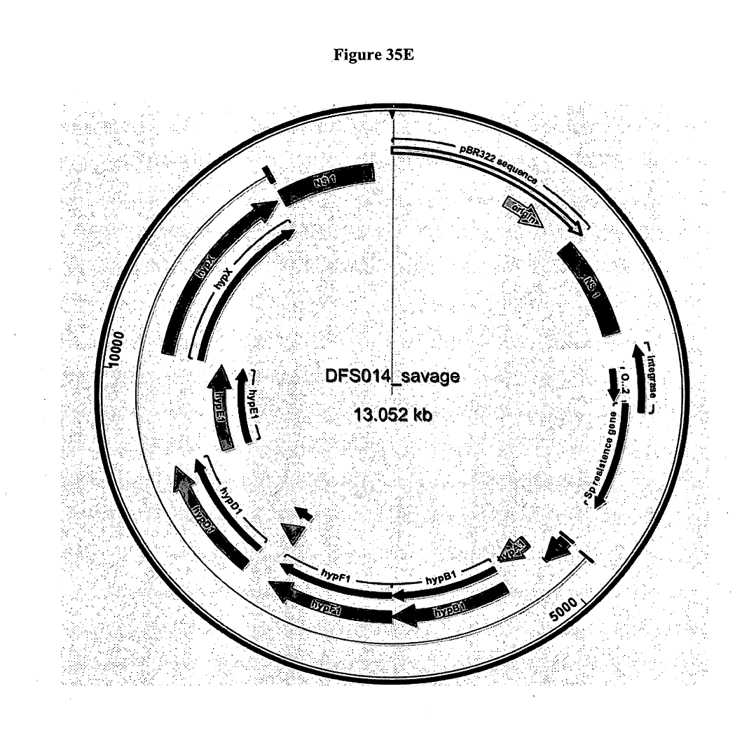

Synechococcus elongatus 7942 was engineered to express hydrogenases as follows. To express the Ralstonia eutropha soluble hydrogenase, plasmids DFS014 and DFS015 were constructed by standard molecular-biological techniques. Plasmid DFS014 (FIG. 35E) contains genes encoding the hydrogenase maturation factors HypB1, HypF1, HypD1, HypE1, and HypX transcribed as a single operon from the E. Coli lactose promoter, and a spectinomycin resistance gene as a separate transcriptional unit. These genes are flanked by DNA of several hundred base pairs on either side from “Neutral Site 1” (NS1), a site in the Synechococcus genome into which exogenous DNA can be integrated without disrupting host cell growth. This plasmid also expresses an integrase to facilitate plasmid integration, as is standard in the Synechococcus integration system.

Plasmid DFS015 (FIG. 35A) contains genes encoding the soluble hydrogenase enzyme subunits HoxF, HoxU, Hoxy and HoxH as well as the factors HoxW and HoxI, transcribed as a single operon from the E. Coli lactose promoter, and a kanamycin resistance gene as a separate transcriptional unit. These genes are flanked by DNA of several hundred base pairs on either side from “Neutral Site 2” (NS2), a site distinct from NS1 in the Synechococcus genome into which exogenous DNA can be integrated without disrupting host cell growth.

Plasmid DFS014 was inserted into the genome of Synechococcus elongatus 7942 by standard techniques, selecting for specinomycin resistance. Plasmid DFS015 was inserted into the genome of the resulting strain by standard techniques, selecting for kanamycin resistance. After each transformation, the structure of the integrated DNA was confirmed by. Southern blot and PCR analysis of junctional regions.

The sequence of the integrated DNA is confirmed by standard techniques. Production of hydrogen is demonstrated by standard techniques, for example using the dithionite/methylviologen assay described herein.

To express the Ralstonia membrane-bound hydrogenase, plasmid DFS018 (FIG. 35B) was constructed by standard molecular-biological techniques.

Plasmid DFS018 contains genes encoding the membrane-bound hydrogenase enzyme subunits HoxK, HoxG, and additional factors HoxZ, HoxM, HoxL, HoxO, HoxQ, HoxT and Hoxy, as well as elements that are present in the interstices between these genes in the natural Ralstonia sequence, transcribed as a single operon from the E. Coli lactose promoter and followed by a ribosomal RNA transcription termination sequence, and a kanamycin resistance gene as a separate transcriptional unit. These genes are flanked by DNA of several hundred base pairs on either side from “Neutral Site 2” (NS2).

Plasmid DFS014 was inserted into the genome of Synechococcus elongatus 7942 by standard techniques, selecting for specinomycin resistance. Plasmid DFS018 was inserted into the genome of the resulting strain by standard techniques, selecting for kanamycin resistance. After each transformation, the structure of the integrated DNA was confirmed by Southern blot and PCR analysis of junctional regions.

The sequence of the integrated DNA is confirmed by standard techniques. Production of hydrogen is demonstrated by standard techniques, for example using the dithionite/methylviologen assay described herein.

In some situations it is preferable to use a plasmid that encodes a variant of the membrane-bound hydrogenase, in which the membrane-binding segment of this hydrogenase is deleted. In such cases, a plasmid analogous to DFS018 is constructed; the construct differs from DFS108 in that sequences encoding Leu310-His360 of hoxK are deleted. This plasmid is then used analogously to DFS018 to construct a Synechococcus derivative as described above.

To express the Chlamydomonas reinhardtii [FeFe], plasmids DFS016 and DFS017 were constructed by standard molecular-biological techniques. Plasmid DFS016 (FIG. 35C) contains genes encoding the hydrogenase HydA and spectinomycin resistance configured analogously to genes in DFS014 described above.

Plasmid DFS017 (FIG. 35D) contains genes encoding the maturation factors HydEF and HydG, and a kanamycin resistance gene configured analogously to genes in DFS018 described above.

Plasmid DFS016 was inserted into the genome of Synechococcus elongatus 7942 by standard techniques, selecting for specinomycin resistance. Plasmid DFS017 was inserted into the genome of the resulting strain by standard techniques, selecting for kanamycin resistance. After each transformation, the structure of the integrated DNA was confirmed by Southern blot and PCR analysis of junctional regions.

The sequence of the integrated DNA is confirmed by standard techniques. Production of hydrogen is demonstrated by standard techniques, for example using the dithionite/methylviologen assay described herein.

Protein levels are assayed with Western blot analysis and can be adjusted as necessary to balance growth and H₂ production. Activity of the complex is assayed in vivo using gas chromatography to measure H₂ production. The hydrogenase complex (or PSI) is optionally conjugated to an affinity tag (e.g., 6× histidine), and the complex is purified to demonstrate in vitro activity. This assay is used to determine efficiency of electron transfer under competition with ferredoxin. The MBH is optionally be substituted with a different O₂ insensitive hydrogenase.

### Example 2

### Construction of a Ferredoxin-Chlamydomonas Hydrogenase Fusion Protein

A ferredoxin-hydrogenase fusion protein is useful to direct the flow of electrons preferentially into a hydrogenase during cellular metabolism. As a result, hydrogen is produced more efficiently from cells. A ferredoxin-hydrogenase fusion protein was designed as follows. The HydA1 [FeFe] hydrogenase of Chlamydomonas reinhardtii and the ‘plant-type’ Fe₂S₂ chloroplast ferredoxin of spinach were chosen as fusion partners. Proteins of these general types interact in photosynthetic cells.

The N-terminus of the Chlamydomonas reinhardtii hydrogenase is close to the docking site for ferredoxin. Experiments were carried out to determine whether fusions of any sort could be tolerated at the N-terminus of the Chlamydomonas hydrogenase without disrupting protein folding or function, and in particular whether such fusions would disrupt docking with ferredoxin, for example, by steric hindrance. A model of the ferredoxin-hydrogenase fusion shows the N-terminus of the hydrogenase buried under the ferredoxin binding site and not accessible for construction of genetic fusions (Chang et. al. [2007] Biophysical J. 93, 3034). Ferredoxin was fused to the N-terminus of the [FeFe] hydrogenase HydA1 in the construction of the fusion proteins described herein.

The ferredoxin and hydrogenase genes were commercially synthesized by Codon Devices, Inc. (Cambridge, Mass.) with codons optimized for expression in yeast, and fused using standard genetic engineering techniques. The fusion protein had a two amino acid threonine-arginine linker at the junction.

The resulting DNA sequence encoding the fusion protein is as follows, with sequences corresponding to ferredoxin underlined and corresponding to SEQ ID NO: 1

ATGGGGCGGCCGCTTCTAGAgaattcgcggccgcttctagagctgcatataaagttactttggtaacaccaacc
ggtaatgtcgaatttcaatgtcctgatgacgtgtacattttagacgccgctgaggaagagggaatagatctacc
atattcttgcagagcaggctcatgttccagttgcgccggtaagcttaaaaactggaagcttgaaccaggatgacc
aatctttcttagatgatgaccagatcgatgaaggctgggttctaacatgtgctgcataccctgtatcagacgtc
ccattgaaactcataaggaggaagaacttacagccactagagctgcaccagccgcagaagctcctttgtctca
tgttcaacaggccttagccgagcttgcaaaaccaaaggatgaccctactagaaaacacgtatgtgtccaagtgg
ccccagctgttagggtagcaattgctgaaacacttggtttggcccctggagcaaccactccaaagcagttagct
gagggcctaagaaggcttggttttgatgaagtgttcgacacattgtttggagccgatttaaccataatggaaga
gggctcagaattgttacatagactaactgaacaccttgaggcacatcctcactccgacgaaccattgcctatgt
tcacaagttgctgtccaggttggatcgctatgttagaaaaaagctatcctgatctaattccatacgtgagctca
tgcaagtcccctcaaatgatgttggccgcaatggttaaaagttatttagctgagaagaaaggtatagccccaaa
ggatatggtaatggtcagcatcatgccatgtaccagaaaacaatctgaagcagacagggattggttttgcgttg
acgctgatcctactcttagacagttggatcatgtgattacaaccgttgagttaggaaatatattcaaggaaaga
ggcatcaacctagccgaacttccagagggtgaatgggacaatcctatgggagtaggttcaggcgcaggtgtctt
gtttggaactacaggcggcgtgatggaagctgctttaaggactgcctacgagctattcaccggtacaccattgc
ctagattatcccttagtgaagttaggggaatggatggtattaaagaaactaacattaccatggtaccagcacct
ggctctaagtttgaggaattgttaaaacatagagctgccgcaagagctgaagccgcagctcacggaacaccagg
tcctctagcatgggacggcggtgctggattcactagcgaggatggtaggggcggcataacattgagagtcgccg
ttgcaaatggattaggtaacgctaaaaagcttatcaccaaaatgcaagccggcgaagcaaagtatgattttgtg
gagattatggcttgtccagccggatgtgttggtggaggcggacaacctagatcaactgacaaagcaataacaca
gaagaggcaagctgccctatacaatttggatgaaaaatccactttaagaagaagtcatgaaaacccatctatca
gggagctttatgacacctacttgggtgaacctttaggtcacaaggcacatgaactattgcacacacattatgta
gctggcgggtcgaggaaaaagatgaaaagaaaactagtagcggccgctgcag

The resulting amino acid sequence encoding the fusion protein sequence is as follows, with sequences corresponding to ferredoxin underlined and corresponding to SEQ ID NO: 2:

EFAAASRAAYKVTLVTPTGNVEFQCPDDVYILDAAEEEGIDLPYSCRAGSCSSCAGKLKTGSLNQDD
QSFLDDDQIDEGWVLICAAYPVSDVTIETHKEEELTATRAAPAAEAPLSHVQQALAELAKPKDDPTR
KHVCVQVAPAVRVAIAETLGLAPGATTPKQLAEGLRRLGFDEVFDTLFGADLTIMEEGSELLHRLTE
HLEAHPHSDEPLPMFTSCCPGWIAMLEKSYPDLIPYVSSCKSPQMMLAAMVKSYLAEKKGIAPKDMV
MVSIMPCTRKQSEADRDWFCVDADPTLRQLDHVITTVELGNIFKERGINLAELPEGEWDNPMGVGSG
AGVLFGTTGGVMEAALRTAYELFTGTPLPRLSLSEVRGMDGIKETNITMVPAPGSKFEELLKHRAAA
RAEAAAHGTPGPLAWDGGAGFTSEDGRGGITLRVAVANGLGNAKKLITKMQAGEAKYDFVEIMAC
PAGCVGGGGQPRSTDKAITQKRQAALYNLDEKSTLRRSHENPSIRELYDTYLGEPLGHKAHELLHTH
YVAGGVEEKDEKKTSSGRC

The resulting fused coding sequences were placed downstream of a T7/lac operon promoter/operator in the Novagen Duet vector system using pETDuet-1 (Novagen Inc., Darmstadt, Germany) that had been modified to delete the histidine tag at the N-terminus. This vector includes an ampicillin resistance marker. In addition, the HydG gene of Chlamydomonas was inserted into the same vector downstream of the second T7/lac operon promoter/operator in pETDuet-1, so that this coding sequence uses the start codon contained within the vector's NdeI site. A separate plasmid that carried the Chlamydomonas HydEF gene was constructed using pACYCDuet-1, which includes a chloramphenicol resistance marker. HydEF and HydG encode factors necessary for maturation of [FeFe] hydrogenases. E. coli BL21 cells were transformed with both of these plasmids.

C. reinhardtii HydEF and HydG coding sequences were also synthesized by a contract DNA synthesis company (Codon Devices, Cambridge, Mass.). Diagrams of these plasmids are shown in FIG. 29.

### Example 3

### Expression and Function of the Ferredoxin-Chlamydomonas Hydrogenase Fusion Protein

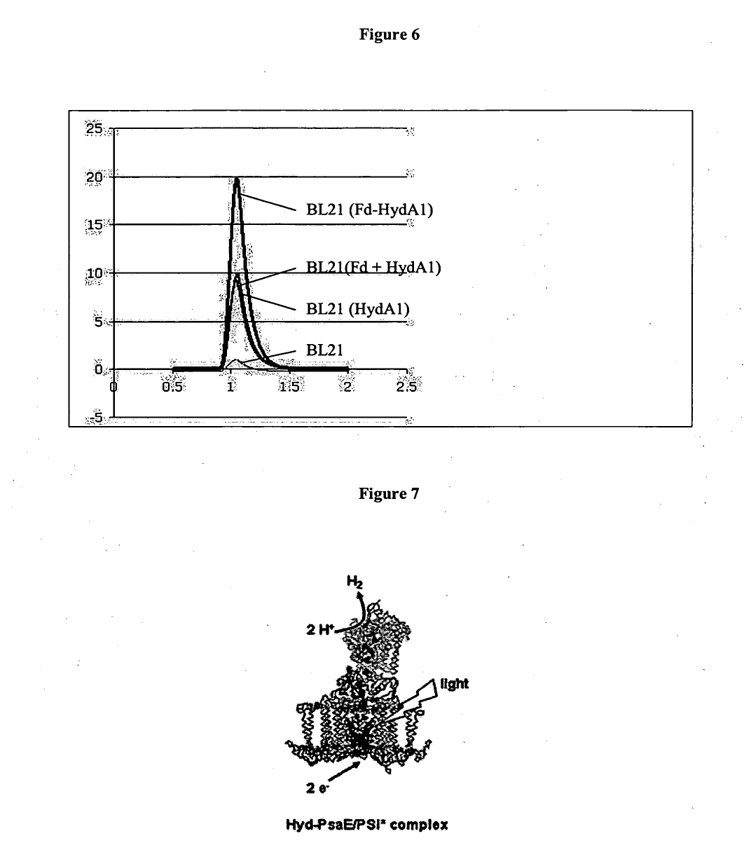

The ferredoxin-hydrogenase protein fusion was functional in vitro when overexpressed in Escherichia coli BL21. The following experiments were performed using an E. coli heterologous expression system similar to that of King et al. (Structure 13:1321-1329, 2005). The cells were grown aerobically until mid-log phase and expression of the genes was induced with isopropyl β-D-1-thiogalactopyranoside (IPTG). The cells were then sparged with argon for several hours to remove any oxygen from the culture. The cells were then lysed in anaerobic conditions, mixed with a buffered solution containing sodium dithionate and methyl viologen and sealed, following a hydrogenase assay procedure described by King et al. (Journal of Bacteriology, 188(6):2163-72, 2006). Sodium dithionite maintains a reduced environment and methyl viologen donates electrons to the hydrogenase and the ferredoxin. After incubation for several hours, the headspace gas was removed with a syringe and analyzed by gas chromatography. The hydrogen peaks on the chromatography trace at one minute after injection are shown in FIG. 6. Extracts of the E. coli strain fusion protein produced significantly more hydrogen than the condition in which the E. coli were without any hydrogenase genes inserted. Extracts of the E. coli strain fusion protein produced significantly more hydrogen than the conditions in which the E. coli either expressed the hydrogenase alone, or in combination, hydrogenase and ferredoxin expressed at the same time, but not fused together.

In the reaction conditions of the cell lysates, methyl viologen donated an electron to either the ferredoxin moiety, the hydrogenase moiety, or both. In lysates of E. coli not expressing an exogenous hydrogenase, a small amount of hydrogen was produced, presumably from the endogenous hydrogenase encoded by E. coli strains. In lysates of E. coli expressing the Chlamydomonas hydrogenase, a significant amount of hydrogen was produced, indicating that the hydrogenase protein was expressed and functional. Additional experiments indicated that expression of the maturation factors HydEF and HydG was essential to produce a functional hydrogenase. In lysates of E. coli expressing the Chlamydomonas hydrogenase and spinach ferredoxin, not fused, the amount of hydrogen produced was about the same as from lysates of E. coli expressing only the Chlamydomonas ferredoxin, indicating that under the dilute conditions of the lysate, the ferredoxin acquires an electron from methyl viologen, but did not interact with the hydrogenase frequently enough to contribute to hydrogen production.

In contrast, in lysates of E. coli expressing spinach ferredoxin fused to Chlamydomonas hydrogenase, the amount of hydrogen produced was greater than, e.g., about twice as much as, the amount yielded from lysates of E. coli expressing only the Chlamydomonas ferredoxin. These results indicated that the ferredoxin-hydrogenase fusion protein functions by absorbing some electrons from methyl viologen through the ferredoxin moiety and then transferring such electrons to the hydrogenase moiety within the same fused molecule. In the fusion protein, the hydrogenase moiety may still have received electrons directly from methyl viologen, but the additional production of hydrogen was due to the presence of the ferredoxin moiety in close proximity.

These results also indicate, unexpectedly, that the N-terminus of a hydrogenase can be used to construct fusion proteins while retaining activity. The experiments also indicate that the C-terminus of a plant-type ferredoxin can be used for construction of an active fusion protein with a hydrogenase. The ferredoxin-hydrogenase fusion protein was found to have enhanced oxygen resistance compared to the parental hydrogenase alone.

Methyl viologen is a man-made chemical dye and is not a natural redox partner of either ferredoxin or hydrogenase. In solution, methyl viologen collides with a molecule containing an iron-sulfur cluster such as a hydrogenase or a ferredoxin and transfers an electron by tunneling when the dye and the iron-sulfur cluster are within a critical distance, which is about 10-14 Angstroms.

In contrast, in a cell, redox reactions between proteins such as ferredoxin, hydrogenase, and other iron-sulfur cluster-containing proteins are accomplished by specific docking events that place the relevant iron-sulfur clusters within a critical distance of each other. As used herein, the term “critical distance” refers to the distance at which the relevant iron-sulfur cluster are able to perform the necessary docking events and redox reactions, which is about 10-14 Angstroms. Ferredoxin is thought to be the major protein carrier of single electrons in cells, and can interact with diverse proteins, while hydrogenases have limited redox partners. Therefore the ferredoxin-hydrogenase fusion protein can be used to channel electron flow into a hydrogenase.

### Example 4

### Expression of Bacterial FeFe Hydrogenases

To demonstrate the generality of the techniques described above, FeFe hydrogenases from the bacteria Clostridium acetobutylicum, Clostridium saccharobutylicum, and Thermotoga maritima were expressed essentially as described above. Specifically, coding sequences for these enzyme were placed into the modified pETDuet-1 vector described above and co-expressed in E. Coli with the maturation factors HydG and HydEF from Chlamydomonas reinhardtii. Expression of the hydrogenase was confirmed by Western blot from versions of the hydrogenases that were expressed with a StrepII epitope tag at the C-terminus of the protein. In each case, the major immunoreactive band was observed at the predicted molecular weight. In addition, a C-terminal fragment of the Clostridium acetobutylicum hydrogenase corresponding to the region homologous to the C. reinhardtii hydrogenase was expressed.

Hydrogenase activity was observed in cell extracts using the dithionite/methylviologen assay as described above, for the Clostridium acetobutylicum, Clostridium saccharobutylicum, and Thermotoga maritima hydrogenases. No hydrogenase activity was observed from E. Coli expressing the C-terminal fragment of the Clostridium acetobutylicum hydrogenase.

### Example 5

### Construction, Expression and Function of Ferredoxin-Bacterial Hydrogenase Fusion Proteins

To demonstrate the generality of the strategy of constructing ferredoxin-hydrogenase fusion proteins, fusions involving ferredoxin and the hydrogenase from Clostridium acetobutylicum were also constructed. The hydrogenase of Clostridium acetobutylicum differs significantly from the hydrogenase of Chlamydomonas reinhardtii in that the Clostridium enzyme has an additional large N-terminal domain that contains two extra Fe₄S₄ and one Fe₂S₂ iron-sulfur clusters, in addition to an Fe4S4 cluster, found in both enzymes, that is adjacent to the FeFe active site. The C. acetobutylicum enzyme also receives electrons from ferredoxin to produce hydrogen, but is significantly more oxygen-resistant than the Clostridium enzyme.

A variety of fusion proteins were constructed, including proteins of the form (N-terminus) ferredoxin-hydrogenase (C-terminus), (N-terminus) hydrogenase-ferredoxin (C-terminus), and (N-terminus) ferredoxin-hydrogenase-ferredoxin (C-terminus). These are termed FH, HF, and FHF proteins respectively. In addition, a fusion protein with a polypeptide linker, ferredoxin-(Gly₄Ser)₄-hydrogenase (an FLH protein), was constructed using the C. acetobutylicum hydrogenase. The amino acid and nucleic acid sequences of these proteins were as follows:

FH protein and nucleic acid sequences using C. acetobutylicum hydrogenase:

(SEQ ID NO: 3)
MGAAASRAAYKVTLVTPTGNVEFQCPDDVYILDAAEEEGIDLPYSCRAGSCSSCAG
KLKTGSLNQDDQSFLDDDQIDEGWVLTCAAYPVSDVTIETHKEEELTATRKTIILNG
NEVHTDKDITILELARENNVDIPTLCFLKDCGNFGKCGVCMVEVEGKGFRAACVA
KVEDGMVINTESDEVKERIKKRVSMLLDKHEFKCGQCSRRENCEFLKLVIKTKAKA
SKPFLPEDKDALVDNRSKAIVIDRSKCVLCGRCVAACKQHTSTCSIQFIKKDGQRAV
GTVDDVCLDDSTCVLLCGQCVIACPVAALKEKSHIEKVQEALNDPKKHVIVAMAPS
VRTAMGELFKMGYGKDVTGKLYTALRMLGFDKVFDINFGADMTIMEEATELLGR
VKNNGPFPMFTSCCPAWVRLAQNYHPELLDNLSSAKSPQQIFGTASKTYYPSISGIA
PEDVYTVTIMPCNDKKYEADIPFMETNSLRDIDASLTTRELAKMIKDAKIKFADLED
GEVDPAMGTYSGAGAIFGATGGVMEAAIRSAKDFAENKELENVDYTEVRGFKGIK
EAEVEIAGNKLNVAVINGASNFFEFMKSGKMNEKQYHFIEVMACPGGCINGGGQP
HVNALDRENVDYRKLRASVLYNQDKNVLSKRKSHDNPAIIKMYDSYFGKPGEGLA
HKLLHVKYTKDKNVSKHETS
(SEQ ID NO: 4)
ATGGGCGCGGCCGCTTCTAGAGCGGCCGCTTCTAGAGCTGCATATAAAGTTACT
TTGGTAACACCAACCGGTAATGTCGAATTTCAATGTCCTGATGACGTGTACATT
TTAGACGCCGCTGAGGAAGAGGGAATAGATCTACCATATTCTTGCAGAGCAGG
CTCATGTTCCAGTTGCGCCGGTAAGCTTAAAACTGGAAGCTTGAACCAGGATGA
CCAATCTTTCTTAGATGATGACCAGATCGATGAAGGCTGGGTTCTAACATGTGC
TGCATACCCTGTATCAGACGTCACCATTGAAACTCATAAGGAGGAAGAACTTAC
AGCCACTAGAAAAACAATAATCTTAAATGGCAATGAAGTGCATACAGATAAAG
ATATTACTATCCTTGAGCTAGCAAGAGAAAATAATGTAGATATCCCAACACTCT
GCTTTTTAAAGGATTGTGGCAATTTTGGAAAATGCGGAGTCTGTATGGTAGAGG
TAGAAGGCAAGGGCTTTAGAGCTGCTTGTGTTGCCAAAGTTGAAGATGGAATG
GTAATAAACACAGAATCCGATGAAGTAAAAGAACGAATCAAAAAAAGAGTTTC
AATGCTTCTTGATAAGCATGAATTTAAATGTGGACAATGTTCTAGAAGAGAAAA
TTGTGAATTCCTTAAACTTGTAATAAAGACAAAAGCAAAAGCTTCAAAACCATT
TTTACCAGAAGATAAGGATGCTCTAGTTGATAATAGAAGTAAGGCTATTGTAAT
TGACAGATCAAAATGTGTACTATGCGGTAGATGCGTAGCTGCATGTAAACAGC
ACACAAGCACTTGCTCAATTCAATTTATTAAAAAAGATGGACAAAGGGCTGTTG
GAACTGTTGATGATGTTTGTCTTGATGACTCAACATGCTTATTATGCGGTCAGTG
TGTAATCGCTTGTCCTGTTGCTGCTTTAAAAGAAAAATCCCATATAGAAAAAGT
TCAAGAAGCTCTTAATGACCCTAAAAAACATGTCATTGTTGCAATGGCTCCATC
AGTAAGAACTGCTATGGGCGAATTATTCAAAATGGGATATGGAAAAGATGTAA
CAGTGAAAACTATATACTGCACTTAGAATGTTAGGCTTTGATAAAGTATTTGATA
AAACTTTGGTGCAGATATGACTATAATGGAAGAAGCTACTGAACTTTTAGGCA
GAGTTAAAAATAATGGCCCATTCCCTATGTTTACATCTTGCTGTCCTGCATGGGT
AAGATTAGCTCAAAATTATCATCCTGAATTATTAGATAATCTTTCATCAGCAAA
ATCACCACAACAAATATTTGGTACTGCATCAAAAACTTACTATCCTTCAATTTC
AGGAATAGCTCCAGAAGATGTTTATACAGTTACTATCATGCCTTGTAATGATAA
AAAATATGAAGCAGATATTCCTTTCATGGAAACTAACAGCTTAAGAGATATTGA
TGCATCCTTAACTACAAGAGAGCTTGCAAAAATGATTAAAGATGCAAAAATTA
AATTTGCAGATCTTGAAGATGGTGAAGTTGATCCTGCTATGGGTACTTACAGTG
GTGCTGGAGCTATCTTTGGTGCAACCGGTGGCGTTATGGAAGCTGCAATAAGAT
CAGCTAAAGACTTTGCTGAAAATAAAGAACTTGAAAATGTTGATTACACTGAA
GTAAGAGGCTTTAAAGGCATAAAAGAAGCGGAAGTTGAAATTGCTGGAAATAA
ACTAAACGTTGCTGTTATAAATGGTGCTTCTAACTTCTTCGAGTTTATGAAATCT
GGAAAAATGAACGAAAAACAATATCACTTTATAGAAGTAATGGCTTGCCCTGG
TGGATGTATAAATGGTGGAGGTCAACCTCACGTAAATGCTCTTGATAGAGAAA
ATGTTGATTACAGAAAACTAAGAGCATCAGTATTATACAACCAAGATAAAAAT
GTTCTTTCAAAGAGAAAGTCACATGATAATCCAGCTATTATTAAAATGTATGAT
AGCTACTTTGGAAAACCAGGTGAAGGACTTGCTCACAAATTACTACACGTAAA
ATACACAAAAGATAAAAATGTTTCAAAACATGAAACTAGTTAA

HF protein and nucleic acid sequences using C. acetobutylicum hydrogenase

(SEQ ID NO: 5)
MGAAASRKTIILNGNEVHTDKDITILELARENNVDIPTLCFLKDCGNFGKCGVCMV
EVEGKGFRAACVAKVEDGMVINTESDEVKERIKKRVSMLLDKHEFKCGQCSRREN
CEFLKLVIKTKAKASKPFLPEDKDALVDNRSKAIVIDRSKCVLCGRCVAACKQHTS
TCSIQFIKKDGQRAVGTVDDVCLDDSTCLLCGQCVIACPVAALKEKSHIEKVQEAL
NDPKKHVIVAMAPSVRTAMGELFKMGYGKDVTGKLYTALRMLGFDKVFDINFGA
DMTIMEEATELLGRVKNNGPFPMFTSCCPAWVRLAQNYHPELLDNLSSAKSPQQIF
GTASKTYYPSISGIAPEDVYTVTIMPCNDKKYEADIPFMETNSLRDIDASLTTRELAK
MIKDAKIKFADLEDGEVDPAMGTYSGAGAIFGATGGVMEAAIRSAKDFAENKELE
NVDYTEVRGFKGIKEAEVEIAGNKLNVAVINGASNFFEFMKSGKMNEKQYHFIEV
MACPGGCINGGGQPHVNALDRENVDYRKLRASVLYNQDKNVLSKRKSHDNPAIIK
MYDSYFGKPGEGLAHKLLHVKYTKDKNVSKHETRAAYKVTLVTPTGNVEFQCPD
DVYILDAAEEEGIDLPYSCRAGSCSSCAGKLKTGSLNQDDQSFLDDDQIDEGWVLT
CAAYPVSDVTIETHKEEELTATS
(SEQ ID NO: 6)
ATGGGCGCGGCCGCTTCTAGAAAAACAATAATCTTAAATGGCAATGAAGTGCA
TACAGATAAAGATATTACTATCCTTGAGCTAGCAAGAGAAAATAATGTAGATAT
CCCAACACTCTGCTTTTTAAAGGATTGTGGCAATTTTGGAAAATGCGGAGTCTG
TATGGTAGAGGTAGAAGGCAAGGGCTTTAGAGCTGCTTGTGTTGCCAAAGTTGA
AGATGGAATGGTAATAAACACAGAATCCGATGAAGTAAAAGAACGAATCAAA
AAAAGAGTTTCAATGCTTCTTGATAAGCATGAATTTAAATGIGGACAATGTTCT
AGAAGAGAAAATTGTGAATTCCTTAAACTTGTAATAAAGACAAAAGCAAAAGC
TTCAAAACCATTTTTACCAGAAGATAAGGATGCTCTAGTTGATAATAGAAGTAA
GGCTATTGTAATTGACAGATCAAAATGTGTACTATGCGGTAGATGCGTAGCTGC
ATGTAAACAGCACACAAGCACTTGCTCAATTCAATTTATTAAAAAAGATGGACA
AAGGGCTGTTGGAACTGTTGATGATGTTTGTCTTGATGACTCAACATGCTTATTA
TGCGGTCAGTGTGTAATCGCTTGTCCTGTTGCTGCTTTAAAAGAAAAATCCCAT
ATAGAAAAAGTTCAAGAAGCTCTTAATGACCCTAAAAAACATGTCATTGTTGCA
ATGGCTCCATCAGTAAGAACTGCTATGGGCGAATTATTCAAAATGGGATATGGA
AAAGATGTAACAGGAAAACTATATACTGCACTTAGAATGTTAGGCTTTGATAAA
GTATTTGATATAAACTTTGGTGCAGATATGACTATAATGGAAGAAGCTACTGAA
CTTTTAGGCAGAGTTAAAAATAATGGCCCATTCCCTATGTTTACATCTTGCTGTC
CTGCATGGGTAAGATTAGCTCAAAATTATCATCCTGAATTATTAGATAATCTTTC
ATCAGCAAAATCACCACAACAAATATTTGGTACTGCATCAAAAACTTACTATCC
TTCAATTTCAGGAATAGCTCCAGAAGATGTTTATACAGTTACTATCATGCCTTGT
AATGATAAAAAATATGAAGCAGATATTCCTTTCATGGAAACTAACAGCTTAAG
AGATATTGATGCATCCTTAACTACAAGAGAGCTTGCAAAAATGATTAAAGATGC
AAAAATTAAATTTGCAGATCTTGAAGATGGTGAAGTTGATCCTGCTATGGGTAC
TTACAGTGGTGCTGGAGCTATCTTTGGTGCAACCGGTGGCGTTATGGAAGCTGC
AATAAGATCAGCTAAAGACTTTGCTGAAAATAAAGAACTTGAAAATGTTGATT
ACACTGAAGTAAGAGGCTTTAAAGGCATAAAAGAAGCGGAAGTTGAAATTGCT
GGAAATAAACTAAACGTTGCTGTTATAAATGGTGCTTCTAACTTCTTCGAGTTT
ATGAAATCTGGAAAAATGAACGAAAAACAATATCACTTTATAGAAGTAATGGC
TTGCCCTGGTGGATGTATAAATGGTGGAGGTCAACCTCACGTAAATGCTCTTGA
TAGAGAAAATGTTGATTACAGAAAACTAAGAGCATCAGTATTATACAACCAAG
ATAAAAATGTTCTTTCAAAGAGAAAGTCACATGATAATCCAGCTATTATTAAAA
TGTATGATAGCTACTTTGGAAAACCAGGTGAAGGACTTGCTCACAAATTACTAC
ACGTAAAATACACAAAAGATAAAAATGTTTCAAAACATGAAACTAGAGCGGCC
GCTTCTAGAGCTGCATATAAAGTTACTTTGGTAACACCAACCGGTAATGTCGAA
TTTCAATGTCCTGATGACGTGTACATTTTAGACGCCGCTGAGGAAGAGGGAATA
GATCTACCATATTCTTGCAGAGCAGGCTCATGTTCCAGTTGCGCCGGTAAGCTT
AAAACTGGAAGCTTGAACCAGGATGACCAATCTTTCTTAGATGATGACCAGATC
GATGAAGGCTGGGTTCTAACATGTGCTGCATACCCTGTATCAGACGTCACCATT
GAAACTCATAAGGAGGAAGAACTTACAGCCACTAGTTAA

FHF protein and nucleic acid sequences using C. acetobutylicum hydrogenase

(SEQ ID NO: 7)
MGAAASRAAYKVTLVTPTGNVEFQCPDDVYILDAAEEEGIDLPYSCRAGSCSSCAG
KLKTGSLNQDDQSFLDDDQIDEGWVLTCAAYPVSDVTIETHKEEELTATRKTIILNG
NEVHTDKDITILELARENNVDIPTLCFLKDCGNFGKCGVCMVEVEGKGFRAACVA
KVEDGMVINTESDEVKERIKKRVSMLLDKHEFKCGQCSRRENCEFLKLVIKTKAKA
SKPFLPEDKDALVDNRSKAIVIDRSKCVLCGRCVAACKQHTSTCSIQFIKKDGQRAV
GTVDDVCLDDSTCLLCGQCVIACPVAALKEKSHIEKVQEALNDPKKHVIVAMAPS
VRTAMGELFKMGYGKDVTGKLYTALRMLGFDKVFDINFGADMTIMEEATELLGR
VKNNGPFPMFTSCCPAWVRLAQNYHPELLDNLSSAKSPQQIFGTASKTYYPSISGIA
PEDVYTVTIMPCNDKKYEADIPFMETNSLRDIDASLTTRELAKMIKDAKIKFADLED
GEVDPAMGTYSGAGAIFGATGGVMEAAIRSAKDFAENKELENVDYTEVRGFKGIK
EAEVEIAGNKLNVAVINGASNFFEFMKSGKMNEKQYHFIEVMACPGGCINGGGQP
HVNALDRENVDYRKLRASVLYNQDKNVLSKRKSHDNPAIIKMYDSYFGKPGEGLA
HKLLHVKYTKDKNVSKHETRAAYKVTLVTPTGNVEFQCPDDVYILDAAEEEGIDL
PYSCRAGSCSSCAGKLKTGSLNQDDQSFLDDDQIDEGWVLTCAAYPVSDVTIETHK
EEELTATS
(SEQ ID NO: 8)
ATGGGCGCGGCCGCTTCTAGAGCGGCCGCTTCTAGAGCTGCATATAAAGTTACT
TTGGTAACACCAACCGGTAATGTCGAATTTCAATGTCCTGATGACGTGTACATT
TTAGACGCCGCTGAGGAAGAGGGAATAGATCTACCATATTCTTGCAGAGCAGG
CTCATGTTCCAGTTGCGCCGGTAAGCTTAAAACTGGAAGCTTGAACCAGGATGA
CCAATCTTTCTTAGATGATGACCAGATCGATGAAGGCTGGGTTCTAACATGTGC
TGCATACCCTGTATCAGACGTCACCATTGAAACTCATAAGGAGGAAGAACTTAC
AGCCACTAGAAAAACAATAATCTTAAATGGCAATGAAGTGCATACAGATAAAG
ATATTACTATCCTTGAGCTAGCAAGAGAAAATAATGTAGATATCCCAACACTCT
GCTTTTTAAAGGATTGTGGCAATTTTGGAAAATGCGGAGTCTGTATGGTAGAGG
TAGAAGGCAAGGGCTTTAGAGCTGCTTGTGTTGCCAAAGTTGAAGATGGAATG
GTAATAAACACAGAATCCGATGAAGTAAAAGAACGAATCAAAAAAAGAGTTTC
AATGCTTCTTGATAAGCATGAATTTAAATGTGGACAATGTTCTAGAAGAGAAAA
TTGTGAATTCCTTAAACTTGTAATAAAGACAAAAGCAAAAGCTTCAAAACCATT
TTTACCAGAAGATAAGGATGCTCTAGTTGATAATAGAAGTAAGGCTATTGTAAT
TGACAGATCAAAATGTGTACTATGCGGTAGATGCGTAGCTGCATGTAAACAGC
ACACAAGCACTTGCTCAATTCAATTTATTAAAAAAGATGGACAAAGGGCTGTTG
GAACTGTTGATGATGTTTGTCTTGATGACTCAACATGCTTATTATGCGGTCAGTG
TGTAATCGCTTGTCCTGTTGCTGCTTTAAAAGAAAAATCCCATATAGAAAAAGT
TCAAGAAGCTCTTAATGACCCTAAAAAACATGTCATTGTTGCAATGGCTCCATC
AGTAAGAACTGCTATGGGCGAATTATTCAAAATGGGATATGGAAAAGATGTAA
CAGGAAAACTATATACTGCACTTAGAATGTTAGGCTTTGATAAAGTATTTGATA
TAAACTTTGGTGCAGATATGACTATAATGGAAGAAGCTACTGAACTTTTAGGCA
GAGTTAAAAATAATGGCCCATTCCCTATGTTTACATCTTGCTGTCCTGCATGGGT
AAGATTAGCTCAAAATTATCATCCTGAATTATTAGATAATCTTTCATCAGCAAA
ATCACCACAACAAATATTTGGTACTGCATCAAAAACTTACTATCCTTCAATTTC
AGGAATAGCTCCAGAAGATGTTTATACAGTTACTATCATGCCTTGTAATGATAA
AAAATATGAAGCAGATATTCCTTTCATGGAAACTAACAGCTTAAGAGATATTGA
TGCATCCTTAACTACAAGAGAGCTTGCAAAAATGATTAAAGATGCAAAAATTA
AATTTGCAGATCTTGAAGATGGTGAAGTTGATCCTGCTATGGGTACTTACAGTG
GTGCTGGAGCTATCTTTGGTGCAACCGGTGGCGTTATGGAAGCTGCAATAAGAT
CAGCTAAAGACTTTGCTGAAAATAAAGAACTTGAAAATGTTGATTACACTGAA
GTAAGAGGCTTTAAAGGCATAAAAGAAGCGGAAGTTGAAATTGCTGGAAATAA
ACTAAACGTTGCTGTTATAAATGGTGCTTCTAACTTCTTCGAGTTTATGAAATCT
GGAAAAATGAACGAAAAACAATATCACTTTATAGAAGTAATGGCTTGCCCTGG
TGGATGTATAAATGGTGGAGGTCAACCTCACGTAAATGCTCTTGATAGAGAAA
ATGTTGATTACAGAAAACTAAGAGCATCAGTATTATACAACCAAGATAAAAAT
GTTCTTTCAAAGAGAAAGTCACATGATAATCCAGCTATTATTAAAATGTATGAT
AGCTACTTTGGAAAACCAGGTGAAGGACTTGCTCACAAATTACTACACGTAAA
ATACACAAAAGATAAAAATGTTTCAAAACATGAAACTAGAGCGGCCGCTTCTA
GAGCTGCATATAAAGTTACTTTGGTAACACCAACCGGTAATGTCGAATTTCAAT
GTCCTGATGACGTGTACATTTTAGACGCCGCTGAGGAAGAGGGAATAGATCTAC
CATATTCTTGCAGAGCAGGCTCATGTTCCAGTTGCGCCGGTAAGCTTAAAACTG
GAAGCTTGAACCAGGATGACCAATCTTTCTTAGATGATGACCAGATCGATGAAG
GCTGGGTTCTAACATGTGCTGCATACCCTGTATCAGACGTCACCATTGAAACTC
ATAAGGAGGAAGAACTTACAGCCACTAGTTAA

FLH protein and nucleic acid sequences using C. acetobutylicum hydrogenase:

(SEQ ID NO: 9)
MGAAASRAAYKVTLVTPTGNVEFQCPDDVYILDAAEEEGIDLPYSCRAGSCSSCAG
KLKTGSLNQDDQSFLDDDQIDEGWVLTCAAYPVSDVTIETHKEEELTATRGGGGSG
GGGSGGGGSGGGGSKTIILNGNEVHTDKDITILELARENNVDIPTLCFLKDCGNFGK
CGVCMVEVEGKGFRAACVAKVEDGMVINTESDEVKERIKKRVSMLLDKHEFKCG
QCSRRENCEFLKLVIKTKAKASKPFLPEDKDALVDNRSKAIVIDRSKCVLCGRCVA
ACKQHTSTCSIQFIKKDGQRAVGTVDDVCLDDSTCLLCGQCVIACPVAALKEKSHI
EKVQEALNDPKKHVIVAMAPSVRTAMGELFKMGYGKDVTGKLYTALRMLGFDK
VFDINFGADMTIMEEATELLGRVKNNGPFPMFTSCCPAWVRLAQNYHPELLDNLSS
AKSPQQIFGTASKTYYPSISGIAPEDVYTVTIMPCNDKKYEADIPFMETNSLRDIDAS
LTTRELAKMIKDAKIKFADLEDGEVDPAMGTYSGAGAIFGATGGVMEAAIRSAKD
FAENKELENVDYTEVRGFKGIKEAEVEIAGNKLNVAVINGASNFFEFMKSGKMNE
KQYHFIEVMACPGGCINGGGQPHVNALDRENVDYRKLRASVLYNQDKNVLSKRK
SHDNPAIIKMYDSYFGKPGEGLAHKLLHVKYTKDKNVSKHETS
(SEQ ID NO: 10)
ATGGGCGCGGCCGCTTCTAGAGCGGCCGCTTCTAGAGCTGCATATAAAGTTACT
TTGGTAACACCAACCGGTAATGTCGAATTTCAATGTCCTGATGACGTGTACATT
TTAGACGCCGCTGAGGAAGAGGGAATAGATCTACCATATTCTTGCAGAGCAGG
CTCATGTTCCAGTTGCGCCGGTAAGCTTAAAACTGGAAGCTTGAACCAGGATGA
CCAATCTTTCTTAGATGATGACCAGATCGATGAAGGCTGGGTTCTAACATGTGC
TGCATACCCTGTATCAGACGTCACCATTGAAACTCATAAGGAGGAAGAACTTAC
AGCCACTAGAGGTGGTGGAGGATCAGGTGGTGGAGGATCAGGTGGTGGAGGAT
CAGGTGGTGGAGGATCAAAAACAATAATCTTAAATGGCAATGAAGTGCATACA
GATAAAGATATTACTATCCTTGAGCTAGCAAGAGAAAATAATGTAGATATCCCA
ACACTCTGCTTTTTAAAGGATTGTGGCAATTTTGGAAAATGCGGAGTCTGTATG
GTAGAGGTAGAAGGCAAGGGCTTTAGAGCTGCTTGTGTTGCCAAAGTTGAAGA
TGGAATGGTAATAAACACAGAATCCGATGAAGTAAAAGAACGAATCAAAAAA
AGAGTTTCAATGCTTCTTGATAAGCATGAATTTAAATGTGGACAATGTTCTAGA
AGAGAAAATTGTGAATTCCTTAAACTTGTAATAAAGACAAAAGCAAAAGCTTC
AAAACCATTTTTACCAGAAGATAAGGATGCTCTAGTTGATAATAGAAGTAAGG
CTATTGTAATTGACAGATCAAAATGTGTACTATGCGGTAGATGCGTAGCTGCAT
GTAAACAGCACACAAGCACTTGCTCAATTCAATTTATTAAAAAAGATGGACAA
AGGGCTGTTGGAACTGTTGATGATGTTTGTCTTGATGACTCAACATGCTTATTAT
GCGGTCAGTGTGTAATCGCTTGTCCTGTTGCTGCTTTAAAAGAAAAATCCCATA
TAGAAAAAGTTCAAGAAGCTCTTAATGACCCTAAAAAACATGTCATTGTTGCAA
TGGCTCCATCAGTAAGAACTGCTATGGGCGAATTATTCAAAATGGGATATGGAA
AAGATGTAACAGGAAAACTATATACTGCACTTAGAATGTTAGGCTTTGATAAAG
TATTTGATATAAACTTTGGTGCAGATATGACTATAATGGAAGAAGCTACTGAAC
TTTTAGGCAGAGTTAAAAATAATGGCCCATTCCCTATGTTTACATCTTGCTGTCC
TGCATGGGTAAGATTAGCTCAAAATTATCATCCTGAATTATTAGATAATCTTTC
ATCAGCAAAATCACCACAACAAATATTTGGTACTGCATCAAAAACTTACTATCC
TTCAATTTCAGGAATAGCTCCAGAAGATGTTTATACAGTTACTATCATGCCTTGT
AATGATAAAAAATATGAAGCAGATATTCCTTTCATGGAAACTAACAGCTTAAG
AGATATTGATGCATCCTTAACTACAAGAGAGCTTGCAAAAATGATTAAAGATGC
AAAAATTAAATTTGCAGATCTTGAAGATGGTGAAGTTGATCCTGCTATGGGTAC
TTACAGTGGTGCTGGAGCTATCTTTGGTGCAACCGGTGGCGTTATGGAAGCTGC
AATAAGATCAGCTAAAGACTTTGCTGAAAATAAAGAACTTGAAAATGTTGATT
ACACTGAAGTAAGAGGCTTTAAAGGCATAAAAGAAGCGGAAGTTGAAATTGCT
GGAAATAAACTAAACGTTGCTGTTATAAATGGTGCTTCTAACTTCTTCGAGTTT
ATGAAATCTGGAAAAATGAACGAAAAACAATATCACTTTATAGAAGTAATGGC
TTGCCCTGGTGGATGTATAAATGGTGGAGGTCAACCTCACGTAAATGCTCTTGA
TAGAGAAAATGTTGATTACAGAAAACTAAGAGCATCAGTATTATACAACCAAG
ATAAAAATGTTCTTTCAAAGAGAAAGTCACATGATAATCCAGCTATTATTAAAA
TGTATGATAGCTACTTTGGAAAACCAGGTGAAGGACTTGCTCACAAATTACTAC
ACGTAAAATACACAAAAGATAAAAATGTTTCAAAACATGAAACTAGTTAA

The FH, HF, FHF and FLH enzymes were expressed in active form essentially as described in Examples 1 and 2. Specifically, coding sequences were obtained from a contract DNA synthesis company essentially as described above, and placed into the pETDuet-1 vector from Example 2 that also contained an E. Coli codon-optimized coding sequence for HydG from Chlamydomonas as described above. Using standard molecular biology techniques, this plasmid was placed into E. Coli along with the pACYCDuet-1 plasmid encoding Chlamydomonas HydEF, to allow for maturation of the hydrogenase. Extracts of cells expressing the FH, HF, FHF and FLH enzymes were tested for hydrogen production as described in Example 3. Hydrogen production was observed from each fusion protein, with similar levels of hydrogen being produced in each case. These results indicate that ferredoxin can be fused to either the N- or C-terminus of this hydrogenase, with or without a linker, and hydrogenase activity is retained.

The expression of proteins of the correct molecular weight that included a hydrogenase, one or more ferredoxins, and a linker, was verified by Western blot. The FH, HF, FHF and FLH proteins, as well as the parental C. acetobutylicum hydrogenase were expressed as described above with and without the StrepII epitope tag from the pETDuet-1 vector. The following molecular weights for the various proteins were observed as follows: hydrogenase alone ˜68,000; FH protein ˜80,000; HF protein ˜80,000; FHF protein ˜91,000; and FLH protein ˜83,000.

### Example 6

### Comparative Analysis of [FeFe]-Hydrogenase Sequences to Identify Oxygen-Resistant Hydrogenases

All currently known [FeFe]-hydrogenases are irreversibly inhibited by oxygen. However, there is a large range of enzymatic half lives between different species. The hydrogenase from the unicellular green algae Chlamydomonas reinhardtii is inactivated in a matter of seconds in the presence of oxygen, while the anaerobic bacterium Clostridium pasteurianum possesses a hydrogenase with a 400-fold higher half-life, e.g. on the order of several minutes. Because Chlamydomonas is an aerobic organism while Clostridium is an obligate anaerobe, this pattern of oxygen sensitivity is surprising and indicates that the oxygen environment of an organism is not positively correlated with the oxygen-resistance of its hydrogenase.

Larger hydrophobic amino acids in the gas channels (such as tryptophan, methionine, phenylalanine) were predicted to be indicators of more oxygen resistant proteins, since they would block oxygen access to the channels but still allow hydrogen access to the active site. These larger amino acids cluster in organisms that live in oxygenated environments. This strategy is supported by the hydrogenases from Ralstonia eutropha, a strictly aerobic organism that lives at the surface of ponds. Selective pressure for oxygen tolerance led its hydrogenases to be entirely insensitive to oxygen. However, many of the hydrogenases with longer half-lives in oxygen are found in strict anaerobes from deep water or pond sediment.

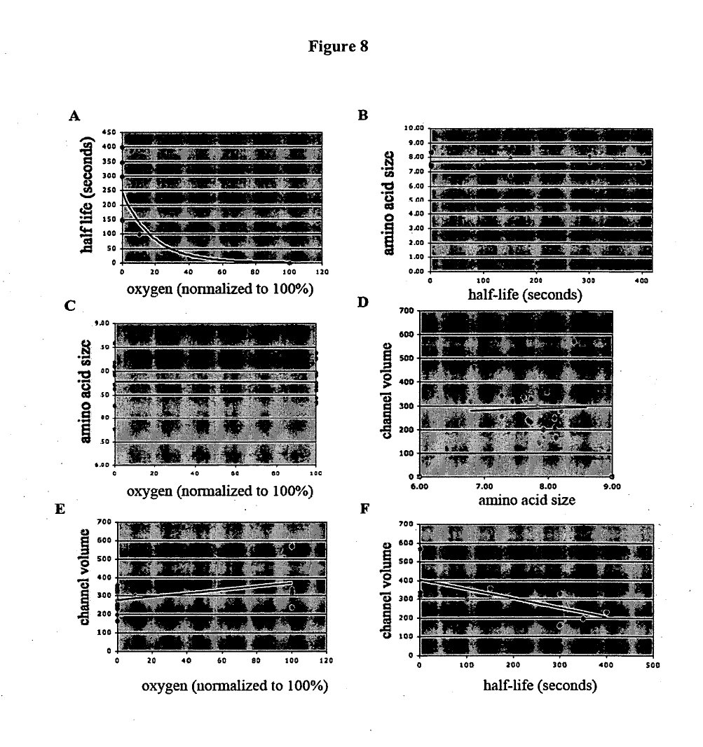

Twenty five [FeFe] hydrogenase sequences were compared. These sequences were found through a TBLASTN search of the NCBI nucleotide database against the protein sequence of the [FeFe]-hydrogenase from Chlamydomonas reinhardtii. The list includes all of the characterized [FeFe]-hydrogenases, as well as proteins annotated as hydrogenases based on sequence homology, from plants, algae, and bacteria. Five of the sequences come from the Sargasso Sea Database (SSDB), a metagenomics project from surface water near Bermuda, and four came from metagenomics of human gut microflora samples. Half-life information is available for a subset of these hydrogenases, including the Chlamydomonas reinhardtii hydrogenase with a half-life of a few seconds, and the Clostridium acetobutylicum hydrogenase with a half-life of several minutes in atmospheric oxygen levels. However, comparisons of the half-life and the amount of oxygen present in the organism's environment show that species that exist within environments with high oxygen concentrations possess hydrogenases whose half-lives in oxygen are significantly shorter than those from anaerobic organisms (FIG. 8A). This analysis indicates that there is a selective pressure for oxygen sensitivity in aerobic organisms. This sensitivity acts as a switch to turn off the hydrogenase when oxygen is present at high levels in order to save the reducing equivalents for aerobic metabolism. Conversely, the relative oxygen-resistance of hydrogenases from anaerobic organisms suggests that these enzymes are not designed to be turned off when oxygen is present, since the organism's metabolism is not designed to use oxygen for an alternative set of pathways.

The gas channel sequences were analyzed by first aligning the sequences using the CLUSTALW algorithm. The gas channel residues were found based on the alignment by identifying the residues that align to the gas channel residues discovered by molecular dynamics simulations of the C. pasteurianum structure (Cohen, J. et al. 2005. Biochemistry Society Transactions 33, 80-82). Each amino acid was then given a score from one to twenty based on its physical size including an estimate of hydration, and the scores were summed over all the residues in the gas channels for each organism and averaged over the number of residues. These numbers were then compared to half-life in the presence of oxygen (FIG. 8B) when such information was available, and oxygen present in the organism's natural environment (FIG. 8C). This analysis showed no correlation between average amino acid size and the oxygen present in the environment.

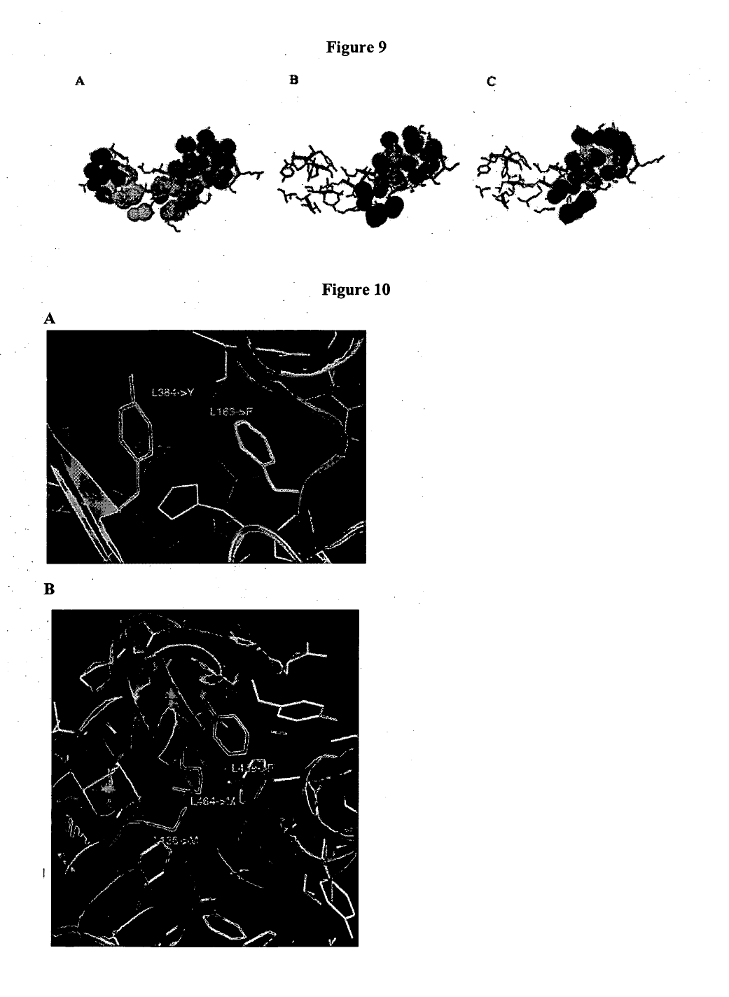

The size of the amino acids may not be the optimal indicator of the actual size of the gas channels. In order to measure the volume of the gas channels, homology models were developed based on alignment to the Clostridium pasteurianum hydrogenase using the SWISSMODEL server (Peters, J. W. 1998. Science 282, 1853-1858). The amino acids identified as gas channel residues by the alignment were separated from the homology model PDB file and used as input into the Computed Atlas of Surface Topography of proteins (CASTp) server (Dundas, J. et al. 2006. Nucleic Acids Research 34, W116-!118). The server uses the Delauny Triangulation to calculate the surface area and volume of voids within the protein structure. Given a PDB file input it returns a structure filled with spheres in the voids it finds (FIG. 9). The calculated volume of the gas channels did not correlate with the average amino acid size, indicating that the protein packing is more complicated than simply being a consequence of the relative sizes of amino acids (FIG. 8D). The gas channel volume, however, correlated slightly with the amount of oxygen present (FIG. 8E) and (more robustly) with the half life of the enzymes (FIG. 8F). To summarize, more oxygen in the environment led to the evolution of larger gas channels. Larger gas channels indicate a shorter half-life.

This analysis of the relationship between oxygen, half-life, volume, and sequence enables identification of better hydrogenases in other organisms and metagenomic datasets. One such metagenomic dataset is that of DeLong et. al., who have sequenced ocean water from different depths, each with well studied physical characteristics including temperature, oxygen concentration, and salinity (Delong, E. F. et al. 2006. Science 311, 496-503). DeLong et al. took samples of ocean water at depths of 10 and 70 (the upper euphotic zone), 130 (the base of the chlorophyll maximum), 200 (below the euphotic zone), 500 (below the upper mesopelagic zone), 700 (in the core of the dissolved oxygen minimum layer) and 4000 meters (in the deep abyss) from ocean water at the Hawaii Ocean Time-series station. By analyzing data from this project as well as comparing [FeFe]-hydrogenase sequences and homology models from environments with different amounts of oxygen, the nature of hydrogenase oxygen tolerance is determined and hydrogenases that are more resistant to oxygen are found. Another dataset useful is that of Warnecke et. al., who sequenced the microbiota of the termite hindgut, a dataset that includes over 100 [FeFe]-hydrogenase sequences separated into ten families, several of which had never before been identified (Warnecke, F. et al. 2007. Nature 450, 560-565).

The net result of these analyses is as follows. Many parameters that might be expected to correlate with oxygen-sensitivity of a hydrogenase do not in fact show such a correlation. A discovery of the invention is that oxygen-sensitivity of a hydrogenase is correlated with the overall volume of the gas channel that is thought to allow escape of hydrogen from the enzyme active site. Based on this discovery, the invention provides a method of enhancing oxygen-resistance of a hydrogenase, which is to decrease the volume of these gas channels. In the specific case of the [FeFe] hydrogenases, there are two channels defined by the following amino acids (Clostridium pasteurianum numbering): Channel A—Ala427, Ala280, Asn464, Phe493, Val284, Ala431, Thr275, Met295, Ala435, Ile461, Ile287, Tyr466, Val468; Channel B—Thr275, Glu278, Glu279, Ala 321, Ile327, Thr330, Ala331, Thr334, Met553, Tyr552, Tyr555, Phe556, Arg563, Ala564, Ile567, Leu568. Decreasing the volume of these channels in a given hydrogenase has the effect of increasing the oxygen resistance of that hydrogenase. This principle is illustrated further below.

### Example 7

### Mutagenesis of hydrogenases for Improved Oxygen Tolerance

Based on the above analysis and examination of the protein structure of hydrogenases, various mutant and fusion protein derivatives of natural hydrogenases were and are designed and constructed.

For testing purposes, a given hydrogenase gene is synthesized for expression in Escherichia coli, although the ultimate use of such a hydrogenase may be in a photosynthetic organism such as Synechococcus. Specifically, a heterologous expression system for hydrogenases, co-expressing a [FeFe] hydrogenase, along with the maturation factors HydEF and HydG is used (King et al. P. W. 2006. Journal of Bacteriology 188, 2163-2172). In general, genes from Chlamydomonas reinhardtii, including maturation factor genes, have a high G-C content and were unstable when expressed in E. coli. By one strategy, this instability was remedied by using the maturation factors from Clostridium acetobutylicum, which has a significantly lower G-C content. Incompatibility of heterologous expression is avoided by purchasing commercially synthesized genes that have been codon optimized to the organism they will be expressed in. This strategy was successfully demonstrated in E. Coli for expression of active C. acetobutylicum hydrogenase in E. Coli. However, this strategy is less convenient because C. acetobutylicum HydE and HydF activities are expressed as separate proteins, so an additional expression construction is necessary. By a second strategy, genes for heterologous expression of the Chlamydomonas reinhardtii hydrogenase in S. cerevisiae (i.e. codon-optimized for expression in yeast) were synthesized and found to be stable and functional in E. coli (see Examples 1 and 2 above). Alternatively, the best genes from the sequence analysis are synthesized with E. coli or Synechococcus codon usage in mind and co-expressed with the maturation factors HydEF and HydG using the Novagen Duet E. coli expression vectors, which allow high-level expression of up to eight proteins at once. Activity of the new [FeFe]-hydrogenase is compared to that of the wild type C. reinhardtii hydrogenase by measuring evolution of hydrogen gas from cell lysates using reduced methyl viologen as an electron carrier and measured using gas chromatography. Half-lives in the presence of oxygen are measured by continuous measurement of hydrogen evolution after oxygen exposure (Vincent, K. A. et al. 2005. Journal of the American Chemical Society 127, 18179-18189; Van der Linden, E. et al. 2004. Journal of Biological Inorganic Chemistry 9, 616-626; Buhrke, T. et al. 2005. Journal of Biological Chemistry 280, 23791-23796).

Molecular dynamics simulations of the [FeFe]-hydrogenase from Clostridium pasteurianum have identified transient hydrophobic channels through which both hydrogen and oxygen gas can penetrate to the active site. Due to its larger size (˜1.6 Å vs. ˜1.35 Å, for Oxygen versus Hydrogen, respectively), oxygen is restricted to only two paths through the protein while hydrogen will more readily diffuse (FIG. 9). The hydrogenase from Chlamydomonas reinhardtii is significantly more sensitive to oxygen than the clostridial hydrogenases, and it was first thought that this is likely because of differences in the gas channels. Sequence comparison and manipulation of the homology model of the Chlamydomonas reinhardtii hydrogenase identified three residues in one of the channels and two in the other that are significantly smaller in C. reinhardtii than in C. pasteurianum. In C. reinhardtii two leucines in gas channel pathway A, at positions 163 and 384, are phenylalanine and tyrosine, respectively (FIG. 10A), and three leucines in pathway B, 136, 464, and 469, are methionine, methionine, and phenylalanine, respectively, in C. pasteurianum (FIG. 10B). These residues are mutated to narrow the width of the gas channel, making it more difficult for oxygen to reach the active site, and increasing the half-life of the C. reinhardtii hydrogenase to be closer to the level of C. pasteurianum. As a result of these manipulations, the following sequence is generated, which is a variant of the C. reinhardtii hydrogenase but with enhanced oxygen resistance:

(SEQ ID NO: 11)
MSALVLKPCAAVSIRGSSCRARQVAPRAPLAASTVRVALATLEAPARRLGNVACAAAAPAAEAPLSHVQQALAELAKPKDDPT

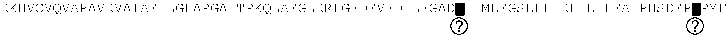

TSCCPGWIAMLEKSYPDLIPYVSSCKSPQMMLAAMVKSYLAEKKGIAPKDMVMVSIMPCTRKQSEADRDWFCVDADPTLRQLD
HVITTVELGNIFKERGINLAELPEGEWDNPMGVGSGAGVLFGTTGGVMEAALRTAYELFTGTPLPRLSLSEVRGMDGIKETNI

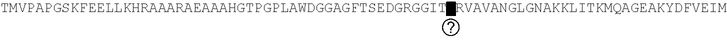

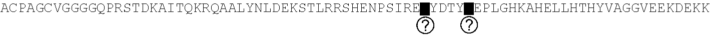

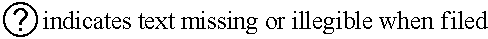

Hydrogenases based on the Chlamydomonas reinhardtii sequence with a subset of these alterations are also useful.

The hydrogenase with the proposed mutations was developed in silico and its gas channel volume was measured as described above. While the gas channel for Chlamydomonas reinhardtii (FIG. 9A) had voids open to oxygen in both channels, the gas channels for Clostridium pasteurianum (FIG. 9B) and the mutated Chlamydomonas reinhardtii (FIG. 9C) have one channel closed off in the static structure. This channel became apparent in molecular dynamics simulations of the protein's natural fluctuations.

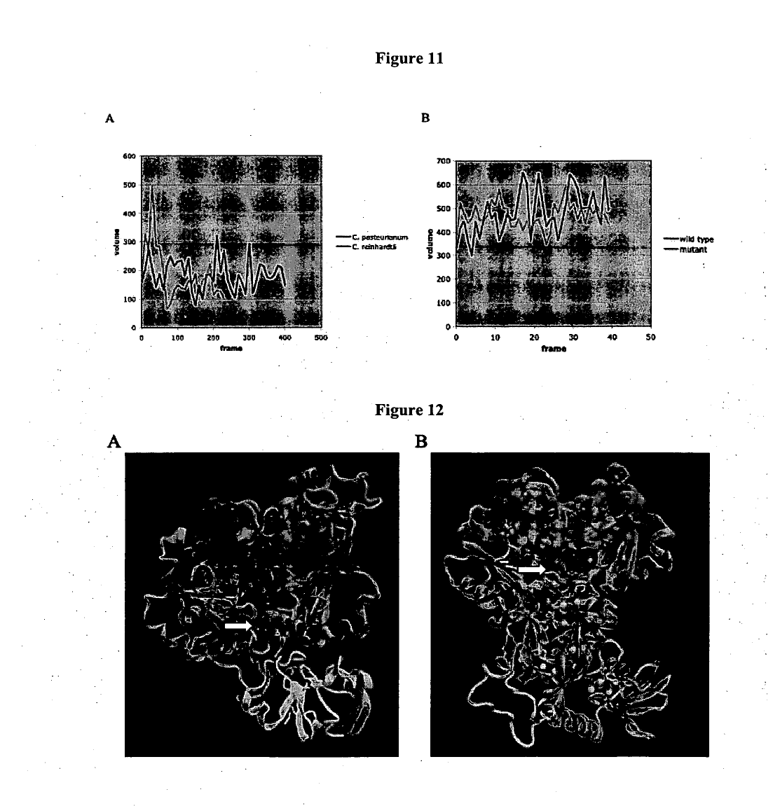

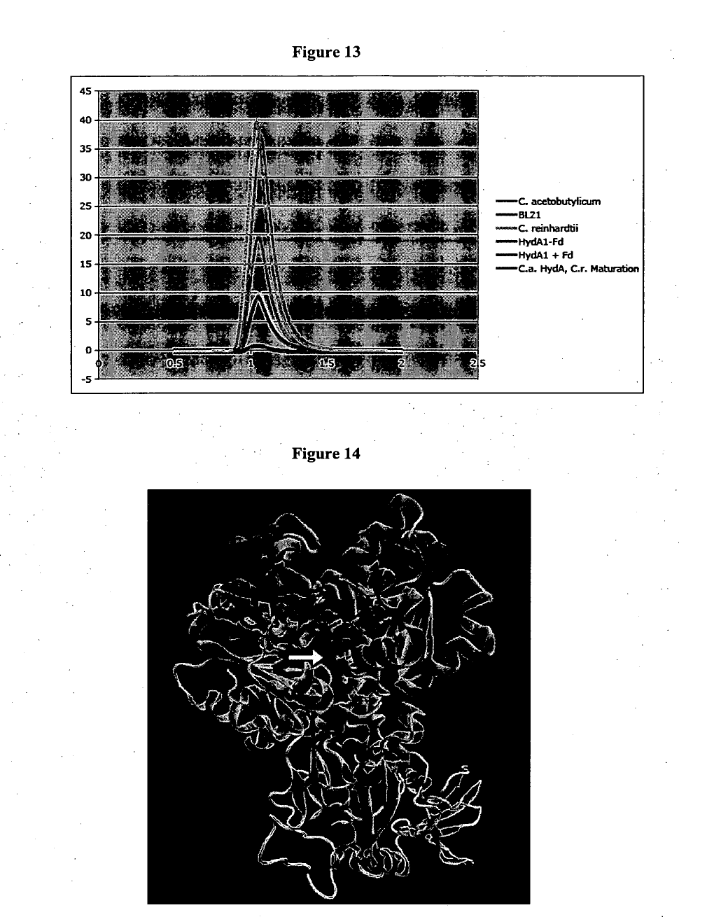

In order to compare the dynamic volume of the gas channels, molecular dynamics simulations of these three structures were performed and gas channel volumes were measured at regular intervals over many frames of the simulation on a femtosecond timescale. The simulations were performed using the NAMD parallel molecular dynamics package and visualized using the VIVID protein structure viewer (Phillips, J. C. et al. Journal of Computational Chemistry 26, 1781-1802, 2005). After a period of initial equilibration for the Chlamydomonas reinhardtii homology model, the volume of the gas channels from pentuply mutated Chlamydomonas hydrogenase and that from Clostridium pasteurianum were remarkably similar (FIG. 11A), with both structures fluctuating around a similar average volume. The same was true for the comparison between the wild type and mutated Chlamydomonas reinhardtii hydrogenases, albeit tested on a shorter time scale (FIG. 11B). Experiments were carried out to determine whether something else is causing the drastic difference in the half lives between these two hydrogenases besides the gas channel volume alone. The C. reinhardtii hydrogenase active site is not completely buried by the protein environment as it is in the C. pasteurianum structure, but is in fact quite close to the protein surface, where it is involved in a direct interaction with ferredoxin for transfer of electrons (FIG. 12). The C. pasteurianum hydrogenase has an extra domain sometimes termed the “ferredoxin-like domain” that electrically connects the active site to the surface through a series of iron-sulfur clusters. Thus, based in part on these in silico analyses and insights but without wishing to be bound by theory, fusing the ferredoxin to the C. reinhardtii hydrogenase at its N-terminus created a protein with blocked access to oxygen and thus enhanced oxygen resistance while still allowing transfer of electrons.

Using methods described in Examples above, expression and hydrogen production of the endogenous C. reinhardtii hydrogenase and spinach ferredoxin proteins, the hydrogenase-ferredoxin fusion protein, and the hydrogenase and ferredoxin proteins expressed separately, were compared to the hydrogenase protein from C. acetobutylicum, with its own maturation factors or with maturation factors of C. reinhardtii, the hydrogenase of C. saccharobutylicum, the hydrogenase of Thermotoga maritima, and the hydrogenase protein of C. reinhardtii, with the latter three using maturation factors from C. reinhardtii. BL21 cells with no hydrogenases expressed were used as a negative control.

The hydrogenase from C. acetobutylicum produced the most hydrogen, followed by C. saccharobutylicum, then C. reinhardtii, with T. maritima producing the least hydrogen. The fusion protein produced a hydrogen yield that was quantitatively between the values of hydrogen production observed for the C. acetobutylicum and C. reinhardtii hydrogenases. Expression of the hydrogenase and ferredoxin, but not fused, produced an amount of hydrogen that was indistinguishable from the amount of hydrogen produced by bacteria transformed by the hydrogenase alone. Moreover, the hydrogen yields of the C. acetobutylicum hydrogenase, expressed with its own maturation factors, and the C. reinhardtii hydrogenase were indistinguishable. These results are the inverse of the results of the King et. al. study (see above).

This assay is used to test other combinations of hydrogenases. Mutagenesis analysis is also performed on the hydrogenase, specifically, to experimentally prove the existence of the gas channels as well as the proton “channel” which have been identified only through computational means.

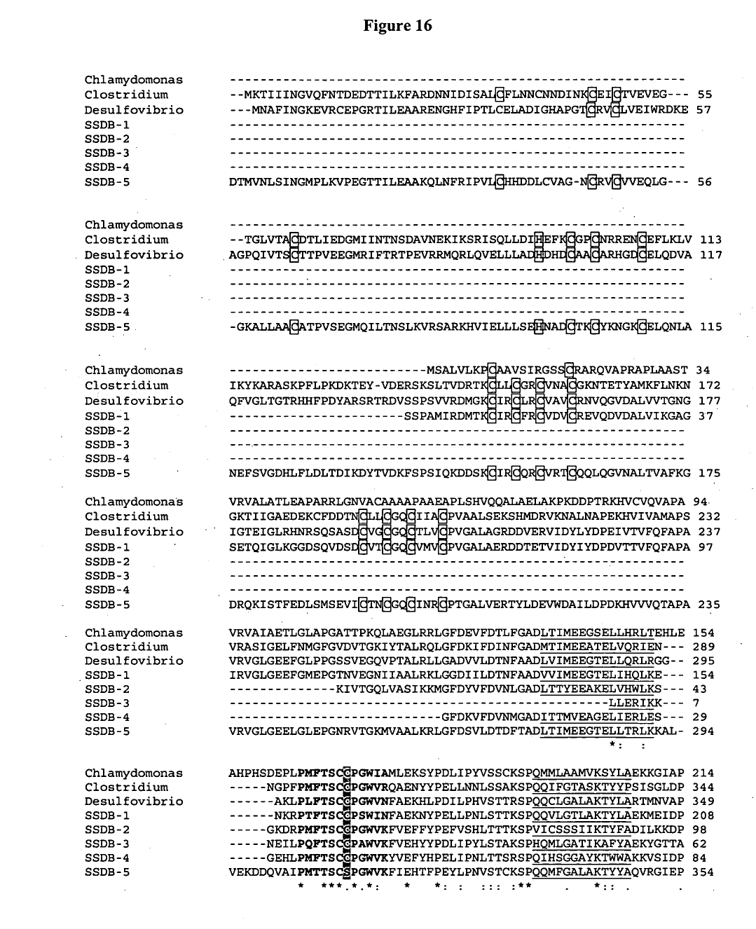

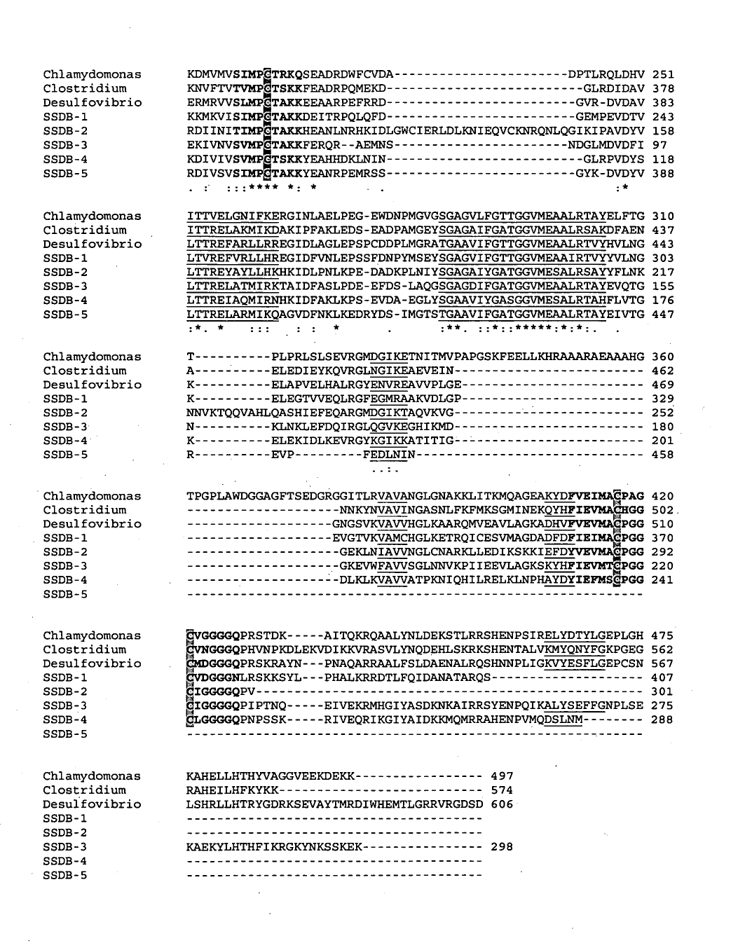

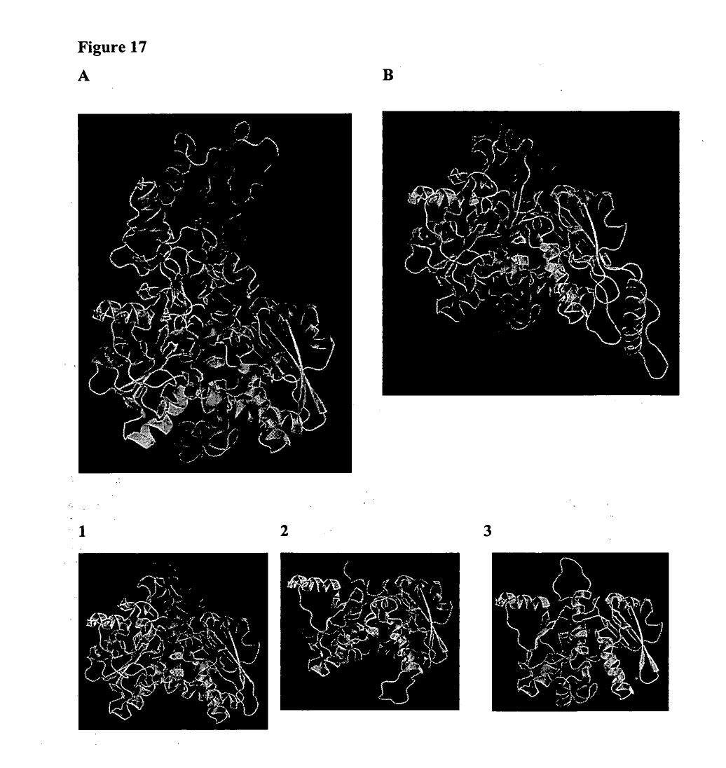

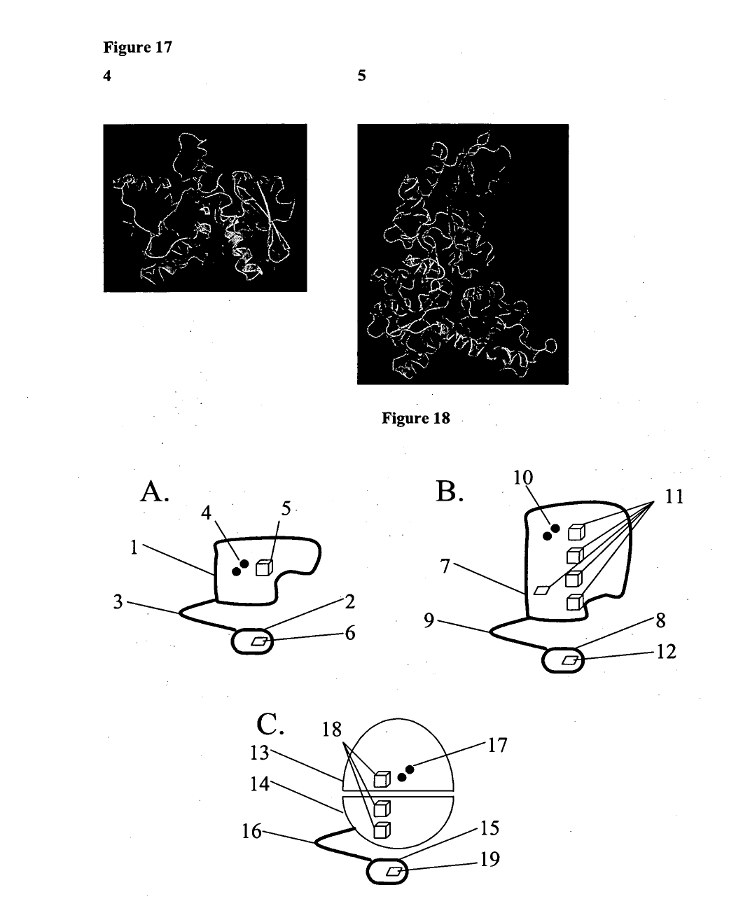

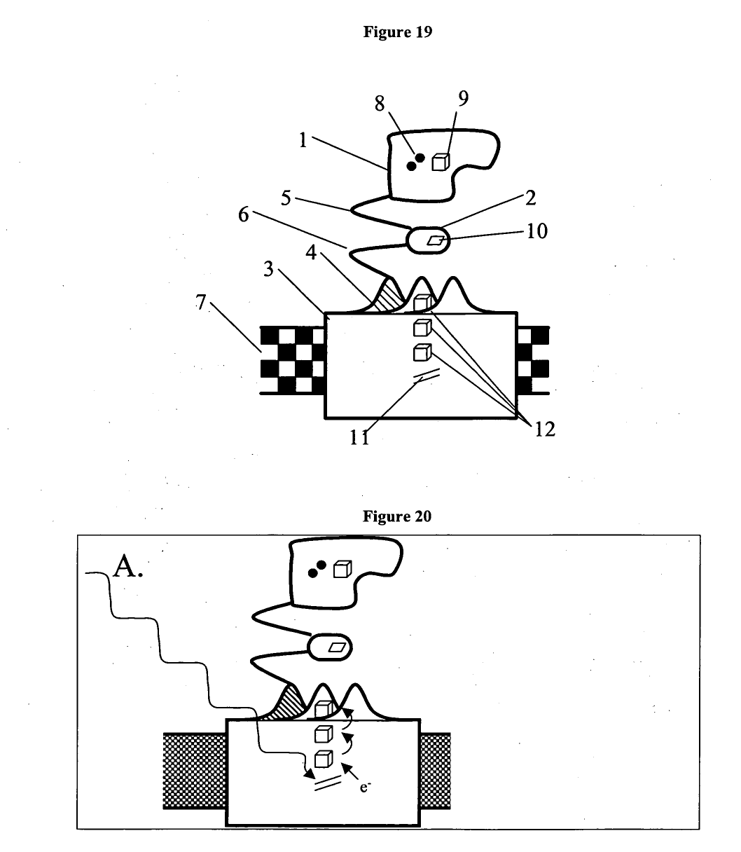

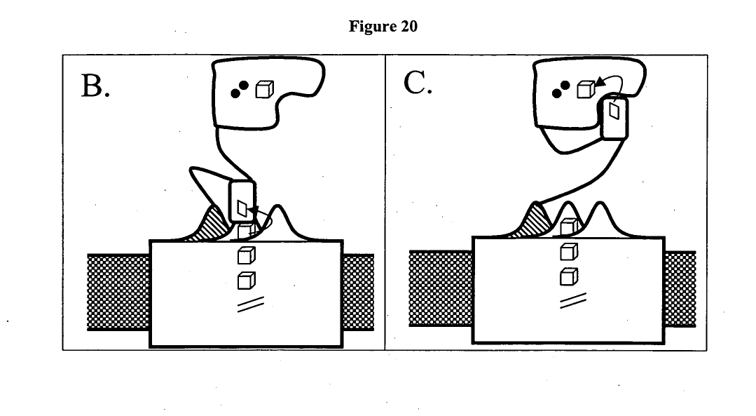

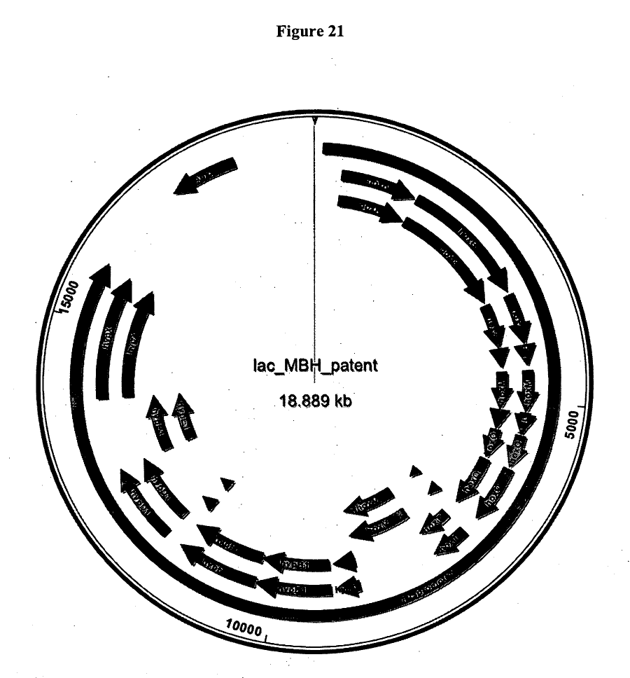

To verify the existence of the gas channels, these channels are blocked by mutagenizing gas channel residues that are invariant between many species, because these are likely to be required for the protein to function. Using the sequence alignment from FIG. 16, the positions were chosen that had the highest and lowest standard deviation in amino acid size. The positions that were the most variable were at the outer edges of the gas channels close to the surface of the protein, whereas the invariant positions were those that were closest to the active site (FIG. 14, FIG. 16). Studies of the gas channels in myoglobin showed that mutating invariant amino acids to larger hydrophobic amino acids blocked the channels and abrogated protein activity (Nagy, et al. 2007. Biotechnology Letters 29, 421-430). By mutating these invariant amino acids and testing for hydrogen production, as well as proper folding and iron cluster integration, it is determined whether or not these channels are required for function and/or how oxygen access is blocked to the active site.

For the proton channels, there are four residues that are believed to act as a chain of hydrogen bond acceptors for protons to pass between as they move from the surface to the active site (Nicolet, Y. et al. 2002. Journal of Inorganic Biochemistry 91, 1-8). These residues are mutagenized and tested for hydrogenase function and pH dependence of the defect. An increased influx of protons improves the catalytic rate of a hydrogenase.

This system is also used for experiments on the maturation of hydrogenases, as well as to analyze the fusion between the hydrogenase and ferredoxin, including overexpression for in vitro studies. This heterologous expression system is also ideal for directed evolution of the hydrogenase for improved oxygen tolerance.

Based on the principles and insights described above and further insights into hydrogenase structure and function, variants of the C. pasteurianum hydrogenase were designed.

Parental Clostridium pasteurianum hydrogenase = SEQ ID NO: 12.

| | |
|---|---|
| MKTIIINGVQFNTDEDTTILKFARDNNIDISALCFLNNCNNDINKCEICTVEVEGTGLVT | 60 |
| ACDTLIEDGMIINTNSDAVNEKIKSRISQLLDTHEFKCGPCNRRENCEFLKLVIKYKARA | 120 |
| SKPFLPKDKTEYVDERSKSLTVDRTKCLLCGRCVNACGKNTETYAMKFLNKNGKTIIGAE | 180 |
| DEKCFDDTNCLLCGQCIIACPVAALSEKSHMDRVKNALNAPEKHVIVAMAPSVRASIGEL | 240 |
| 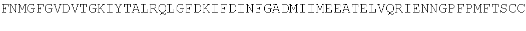 | 300 |
| PGWVRQAENYYPELLNNLSSAKSPQQIFGTASKTYYPSISGLDPKNVFTVTVMPCTSKKF | 360 |
| EADRPQMEKDGLRDIDAVITTRELAKMIKDAKIPFAKLEDSEADPAMGEYSGAGAIFGAT | 420 |
| 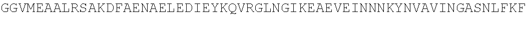 | 480 |
| 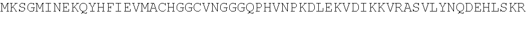 | 540 |
| KSHENTALVKMYQNYFGKPGEGRAHEILHFYKK | |

(SEQ ID NO: 13)
Clostridium pasteurianum hydrogenase with mutations at Ala431Val, Ala435Leu,
Val284Ile, Thr275Val, Phe493Tyr

| | |
|---|---|
| MKTIIINGVQFNTDEDTTILKFARDNNIDISALCFLNNCNNDINKCEICTVEVEGTGLVT | 60 |
| ACDTLIEDGMIINTNSDAVNEKIKSRISQLLDIHEFKCGPCNRRENCEFLKLVIKYKARA | 120 |
| SKPFLPKDKTEYVDERSKSLTVDRTKCLLCGRCVNACGKNTETYAMKFLNKNGKTIIGAE | 180 |
| DEKCFDDTNCLLCGQCIIACPVAALSEKSHMDRVKNALNAPEKHVIVAMAPSVRASIGEL | 240 |
| 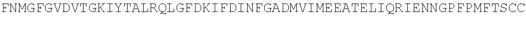 | 300 |
| PGWVRQAENYYPELLNNLSSAKSPQQIFGTASKTYYPSISGLDPKNVFTVTVMPCTSKKF | 360 |
| EADRPQMEKDGLRDIDAVITTRELAKMIKDAKIPFAKLEDSEADPAMGEYSGAGAIFGAT | 420 |
| 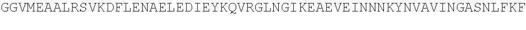 | 480 |
| 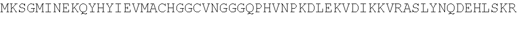 | 540 |
| KSHENTALVKMYQNYFGKPGEGRAHEILHFKYKK | |

(SEQ ID NO: 14)
Clostridium pasteurianum hydrogenase with mutations at Ala431Val, Ala435Leu,
Val284Ile, Thr275Val, Phe493Tyr AND Asn462Arg, Asn289Gly and also Val468Phe

| | |
|---|---|
| MKTIIINGVQFNTDEDTTILKFARDNNIDISALCFLNNCNNDINKCEICTVEVEGTGLVT | 60 |
| ACDTLIEDGMIINTNSDAVNEKIKSRISQLLDIHEFKCGPCNRRENCEFLKLVIKYKARA | 120 |
| SKPFLPKDKTEYVDERSKSLTVDRTKCLLCGRCVNACGKNTETYAMKFLNKNGKTIIGAE | 180 |
| DEKCFDDTNCLLCGQCIIACPVAALSEKSHMDRVKNALNAPEKHVIVAMAPSVRASIGEL | 240 |
| 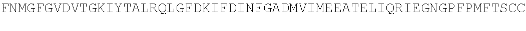 | 300 |
| PGWVRQAENYYPELLNNLSSAKSPQQIFGTASKTYYPSISGLDPKNVFTVTVMPCTSKKF | 360 |
| EADRPQMEKDGLRDIDAVITTRELAKMIKDAKIPFAKLEDSEADPAMGEYSGAGAIFGAT | 420 |
| 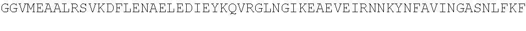 | 480 |
| 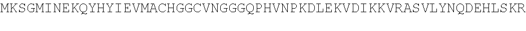 | 540 |
| KSHENTALVKMYQNYFGKPGEGRAHEILHFKYKK | |

Variants of the C. acetobutylicum hydrogenase with the following changes, alone or in combination, are also useful as variants with enhanced oxygen resistance: Thr274Val, Ala279Ser, Val286Leu, Ala426Ser, Ala430Val, Ala434Phe, Ile460Leu, Asn463Lys or Arg, Leu465Trp or Tyr, Val467Phe, Phe492Tyr. The mutation Asn463Lys or Arg is particularly useful if position 287 is glutamate.

### Example 8

### Directed Evolution of the [FeFe]-Hydrogenase from Chlamydomonas reinhardtii

Enzymes have been evolved to recognize different substrates, have improved thermal and oxidative stability, or increased enantioselectivity. It has even been shown that multiple enzyme characteristics can be changed at once (Ness, J. E. et al. 1999. Nature Biotechnology 17, 893-896). Iterative rounds of directed evolution of the hydrogenase enzyme from Clostridium acetobutylicum with increasing levels of oxygen present in the environment is expected to produce an enzyme that is significantly more oxygen tolerant than wild type.

Hydrogenases are reversible enzymes, able to both reduce and oxidize hydrogen and improved oxygen tolerance can be achieved through screens incorporating selective pressure for uptake and oxidation of hydrogen in Chlamyodomonas reinhardtii. However, previous investigators were unable to screen a large number of mutants and have not yielded any significant results. A selection strategy in Escherichia coli permits testing of millions of mutants in an efficient high-throughput manner.

The selection relies on the ferredoxin dependent iron-sulfur flavoprotein glutamate synthase (GlsF) from Synechococcus sp. PCC 7942. The homologous gene from the highly similar cyanobacterial species, Synechocystis sp. PCC 6803 has been shown to be functionally expressed in E. coli, although it does not complement the E. coli glutamate auxotrophy (Navarro, F. et al. 2000. Archives of Biochemistry and Biophysics 379, 267-276), because the endogenous E. coli ferredoxins cannot interact with the natural partners of the photosynthetic ferredoxins. A novel biochemical pathway is created, in which the GlsF gene product is reduced by ferredoxin, which is in turn reduced by the hydrogenase breaking down hydrogen from the environment. This pathway complements the E. coli glutamate auxotrophy (caused by knocking out the glutamate synthase and glutamate dehydrogenase genes) anaerobically and is used to select for oxygen tolerant hydrogenase mutants in the presence of increasing concentrations of oxygen.

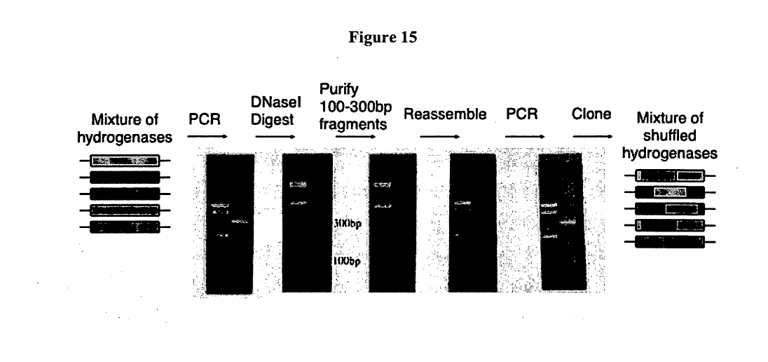

The mutagenesis of the hydrogenase gene employs the family shuffling technique common in directed evolution experiments. Family DNA shuffling is a method for in vitro homologous recombination that combinatorially reassembles Dnasel fragmented genes using error-prone PCR. It has been shown that this method of iterative homologous recombination between closely related genes is critical for sequence evolution (Farinas, et al. 2001. Current Opinion in Biotechnology 12, 545-551; Stemmer, W. P. 1994. Nature 370, 389-391). A library of hydrogenases has already been made this way from six different hydrogenases, although no selection was performed (Nagy, L. E. et al. 2007. Biotechnology Letters 29, 421-430). The C. reinhardtii hydrogenase, as well as the hydrogenases from C. acetobutylicum, Clostridium saccharobutylicum, and the hydrogenases synthesized for use in Example 4 are used. The genes are digested, reassembled with PCR, and cloned into a Novagen Duet vector for coexpression with the maturation factors from C. reinhardtii (FIG. 15).

In the event that the evolution of an entirely oxygen insensitive variant does not occur, the directed evolution method produces a hydrogenase with significantly improved oxygen tolerance than the C. reinhardtii enzyme. Looking at the sequences of the hydrogenases at each round of evolution provides insight into the nature of the oxygen insensitivity. Previous work on oxygen sensitivity has focused on the gas channels. The mutations that improve oxygen tolerance cluster in these regions and the more oxygen tolerant variants have the extra ferredoxin-like domain covering their active site.

### Example 9

### Expression and Function of a Ferredoxin-Hydrogenase Fusion Protein in Synechococcus

The experiments shown herein provide an example of how a ferredoxin-hydrogenase fusion protein is used to direct enhanced hydrogen production in a bacterium. Specifically, the bacterium Synechococcus is used, however, other organisms are also used. Other species include, but are not limited to, cyanobacteria, Clostrium species, and E. coli.

To express the ferredoxin-hydrogenase fusion protein in Synechococcus, an expression vector comprising a promoter, a coding sequence encoding the ferredoxin-hydrogenase fusion protein, a detectable or measurable marker for selection of Synechococcus transformants (such as an antibiotic resistance gene), and a sequence to direct homologous recombination of the plasmid into a ‘neutral site’ in Synechococcus. As used herein, the term “neutral site” is meant to describe a position within the genome of a host organism at which insertion of an exogenous sequence by standard means does not disrupt a required function of that host, e.g. does not compromise the ability of that host to survive or thrive.

A number of specific hydrogenase proteins are used, depending upon the application and conditions. [FeFe] hydrogenase proteins are preferred, however, [NiFe] hydrogenases are also used. Exemplary preferred hydrogenase proteins include the Chlamydomonas hydrogenase, as described above, or a relatively oxygen-resistant hydrogenase, such as the hydrogenase from either Clostridium africanus or from Thermotoga neapolitana, or a relatively oxygen-resistant hydrogenase that is isolated by engineering of a natural hydrogenase. Relevant maturation factors are expressed in the same organism regardless of the source of the hydrogenase used.

To verify expression of the transgene, the use of Synechococcus elongatus 7942, which lacks any endogenous hydrogenase, is preferred. Hydrogenase activity is detected in cell lysates by the methyl viologen assay, which is performed essentially as described above for an E. coli extract. Expression of the hydrogenase is verified by Western blot detection of the epitope tag that is placed at the N- or C-terminus of the fusion protein. Photosynthetically directed production of hydrogen is achieved by growing Synechococcus is grown under standard conditions.

### Example 10

### Construction of Synechococcus Strains with Reduced or Absent Plant-Type Ferredoxin Activity

To enhance production of hydrogen in a photosynthetic organism expressing a ferredoxin-hydrogenase fusion protein, the endogenous ferredoxin in the cell is reduced. For example, in Synechococcus elongatus 7942, there are three Fe₂S₂ ferredoxins encoded at positions 333517-333834, 1548631-1548930, and 2667018-2667386 in the sequenced genome. (See Genome ID 10645 of the NCBI Entrez Genome Project). Each of these genes are knocked out by standard techniques for engineering Synechococcus (Mackey S R, Ditty J L, Clerico E M, Golden S S. Methods Mol. Biol. 2007; 362:115-29). These knockouts are performed in a strain that already expresses a ferredoxin-hydrogenase fusion protein, so that there is always an active ferredoxin in the cell. The resulting cell produces hydrogen in a manner driven by sunlight under standard growth conditions, especially when oxygen is sparged from the medium.

Because the Synechococcus metabolism generally depends on photosynthesis, and Fe₂S₂ ferredoxins are the only means of obtaining electrons from Photosystem I for redox reactions, channeling all of the photosynthetically derived electrons into hydrogen production may be deleterious to cell growth. A linker is placed between the ferredoxin and the hydrogenase. The length of the linker is varied wherein lengthening of the linker region progressively leads to a reduction in the rate of interaction between the ferredoxin and the hydrogenase. Thus, lengthening the linker region allows more electrons to be diverted to other cellular purposes, such as NAD(P)⁺ reduction. Linker region lengths are increased or decreased dependent upon the metabolic needs of the photosynthetic organisms used. Linker region lengths range from 2 amino acids or 22.5 angstroms to 25 amino acids or 225 angstroms.

### Example 11

### Construction of a Photosystem-Ferredoxin-Hydrogenase Fusion Protein

A photosystem-ferredoxin-hydrogenase multiprotein complex is constructed as follows. By way of example, the cyanobacterium Synechococcus elongatus 7942 is used as a host. It is recognized by those skilled in the art that many other hosts can be used, including, but not limited to, other cyanobacteria such as Synechococcus elongatus 6803 or other Synechococcus species, Synechocystis species, various Prochlorococcus species, various Anabaena species such as Anabaena variabilis, various Nostoc species such as Nostoc sp. PCC7120, wild cyanobacteria isolated directly from fresh or salty bodies of water, as well as green algae such as Chlamydomonas or green plants such as Arabidopsis, and corn.

A photosystem-ferredoxin-hydrogenase multiprotein complex has properties that are distinct from individual hydrogenase or photosystem proteins, and which vary from complex to complex depending on the precise configuration. Therefore, in the illustrations below, various complexes are described with distinct names. There are multiple configurations for a photosystem-ferredoxin-hydrogenase multiprotein complex. First, either an [FeFe] hydrogenase or an [NiFe] hydrogenase is used. As a fusion junction within an [FeFe] hydrogenase, which generally has a single subunit, the N-terminus alone, the C-terminus alone, or both termini together are used. As a fusion junction within an [NiFe] hydrogenase, which generally has two subunits, either the N-terminus or C-terminus of either subunit is used. As a fusion junction of the ferredoxin moiety, either the N-terminus or C-terminus, or both termini are used.

Photosystems I and II each contain a large number of proteins, and in principle, an N-terminus or C-terminus of any of these proteins is used as a fusion junction. Within Photosystem I, the N- and C-termini of the proteins PsaC, PsaD, and PsaE are preferred junction sites. The N-terminus of PsaA and PsaB are used, as well as the C-terminus of PsaF and/or Psal, the N-terminus of PsaL, the C-terminus of PsaM, and/or the N-terminus of PsaX. The 1JB0 structure of Photosystem I from Synechocystis 6803 shows the above-mentioned termini and illustrates the spatial relationships of the multiple proteins involved in this complex, see Jordan, P., Fromme, P., Witt, H. T., Klukas, O., Saenger, W., Krauss, N. (2001) NATURE 411: 909-917.

In one particular configuration, a hybrid gene comprising, in an N-terminal to C-terminal direction: a hydrogenase, which may for example be from Ralstonia eutropha, Chlamydomonas, Clostridium, or any other species; a first linker, optionally consisting primarily of glycine and serine; a ‘plant-type’ ferredoxin which may be from a cyanobacterium, a green algae, or a green plant such as spinach, or any other photosynthetic organism; a second linker consisting primarily of glycine and serine; and a gene encoding a photosystem component such as the psaE gene of Synechococcus 7942 is constructed. This configuration is termed the HLFLP (ydrogenase-linker-ferredoxin-linker-photosystem) configuration. An active form of a protein complex including such a hybrid protein is termed a HLFLPase. The hydrogenase-ferredoxin-psaE gene is placed in an expression vector operably linked to a promoter and a marker for selection in Synechococcus, and optionally a region of genetic homology to a ‘neutral site’; namely a site in the Synechococcus genome that can tolerate insertions with no deleterious effects on growth (Mackey S R, Ditty J L, Clerico E M, Golden S S. Methods Mol. Biol. 2007; 362:115-29). The expression vector is placed into Synechococcus 7942 strain that may optionally contain a mutation such as a knockout in the endogenous psaE gene, as well as other mutations such as knockouts of various ferredoxin genes. Details of the vector construction are given below in Example 12.

### Example 12

### Construction of a HLFLPase Using an Oxygen-Resistant Hydrogenase

The HLFLP construction is formed using either an [FeFe] hydrogenase or an NiFe hydrogenase. Because these two classes of hydrogenases have evolved separately and show no sequence or structural similarity, the details of designing an HLFLPase are different for each type of hydrogenase.

In a particular version of an HLFLPase, a derivative of the membrane-bound hydrogenase (MBH) of Rastonia eutropha H16 is used. This hydrogenase has the advantage that it is resistant to atmospheric levels of oxygen. A number of maturation factors are required for this protein to fold and function in its active state. Genes encoding this hydrogenase and its maturation factors are found in the Ralstonia eutropha H16 plasmid pHG1 at coordinates 115 to 15474. To prepare a DNA segment suitable for expressing the Ralstonia membrane-bound hydrogenase in Synechococcus and for construction of an HLFLPase, the following procedures were followed.

First, genomic DNA from Ralstonia was prepared according to standard procedures using a Qiagen bacterial genomic isolation kit, and amplified by PCR using the following primers:

Forward primer:
(SEQ ID NO: 15)
5′ AT GGGCCC ACTAGT gtcgaaacattttatgaagtcatgcg 3′
Reverse primer:
(SEQ ID NO: 16)
5′ AT AAGCTT TCTAGA tcaagatcgtttccccgc 3′

Within these primers, the underlined sequences correspond to Ralstonia DNA, and the flanking 5′ sequences contain restriction enzyme sites ApaI-SpeI and XbaI-HindIII respectively. The resulting amplified product was inserted into the DSBB001 vector containing an E. coli lac promoter cut with XbaI/HindIII. The promoter-MBH synthetic operon was subcloned by excising with ApaI/XbaI and ligated into the Synechococcus integration vector DS1579.

Synechococcus elongatus 7942 was transformed with the YYY-ReMBH expression vector according to standard procedures (Mackey S R, Ditty J L, Clerico E M, Golden S S. Methods Mol. Biol. 2007; 362:115-29), selecting for kanamycin resistance. The structure and function of transformants were verified by Southern blot and tested for the presence of hydrogenase activity in a standard assay (see Example 13; essentially as described above in Example 2).

### Example 13

### Design and Construction of an HLFLPase Using the Ralstonia Membrane-Bound Hydrogenase

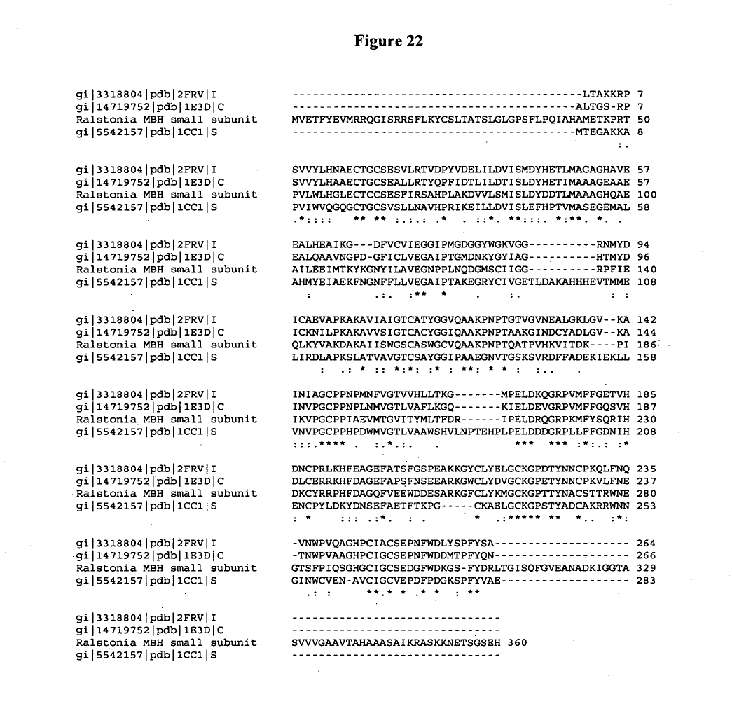

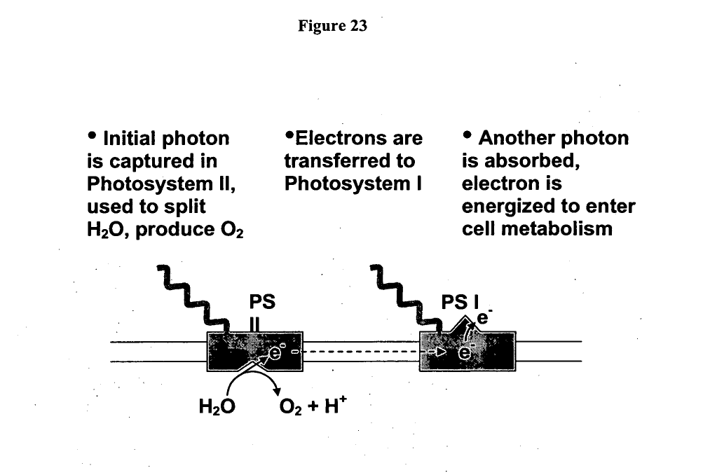

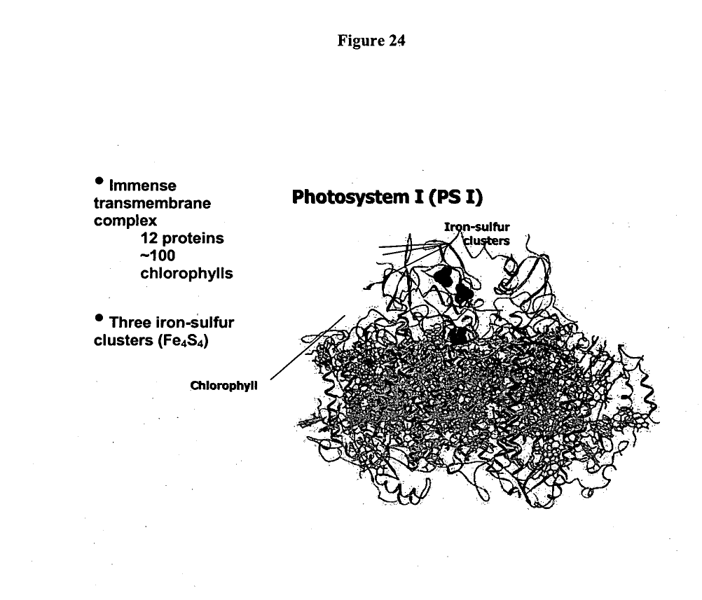

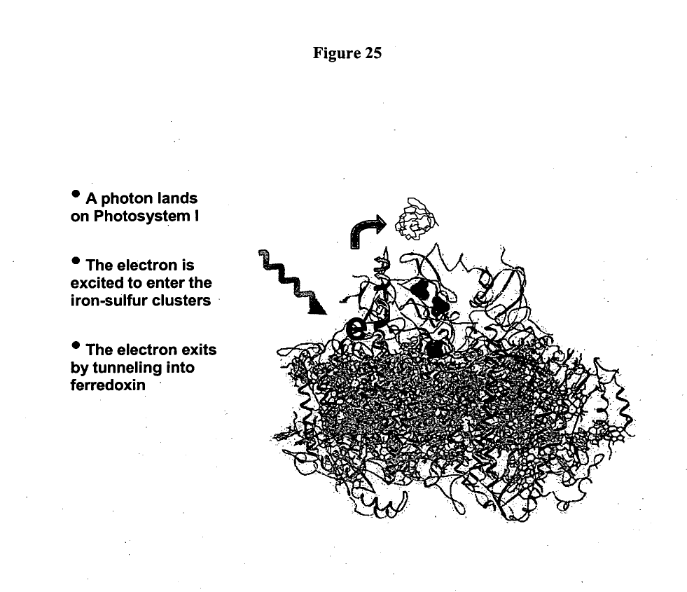

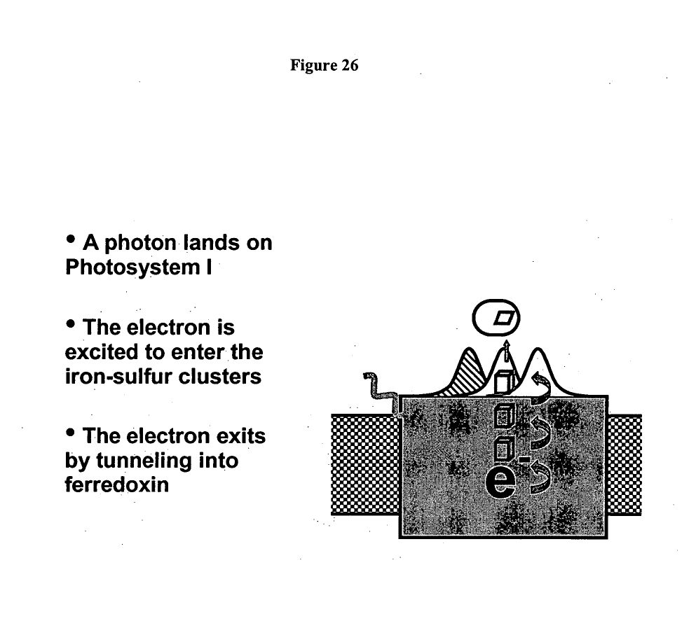

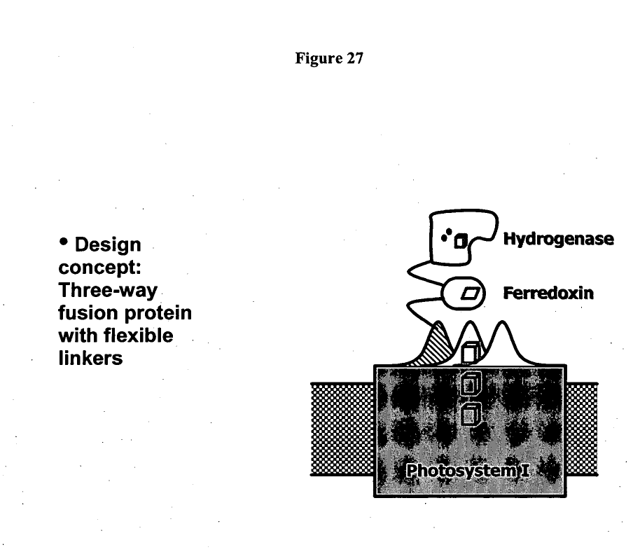

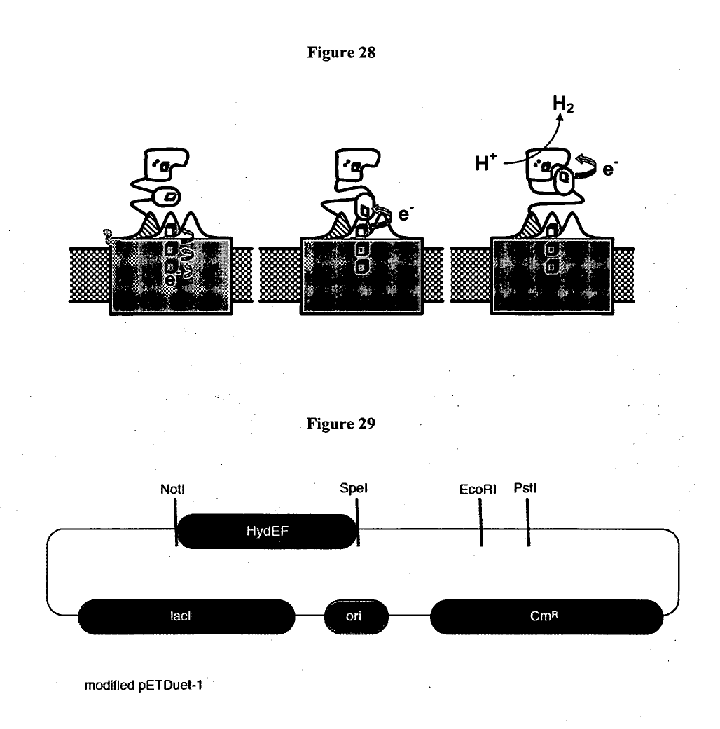

An HLFLPase containing a novel fusion protein comprising the Ralstonia membrane-bound hydrogenase, spinach ferredoxin, and the PsaE protein of S. elongatus 7942 was designed as follows. The Ralstonia MBH is similar in sequence and presumably in three-dimensional structure to the Desulfovibrio [NiFe] hydrogenase for which structures have been determined by X-ray crystallography (Volbeda, A., Charon, M. H., Piras, C., Hatchikian, E. C., Frey, M., Fontecilla-Camps, J. C. (1995) Nature 373: 580-587). Such structures include 2FRV from D. gigas, 1E3D from D. desulfuricans, and 1CC1 from D. baculatum. An alignment of the small subunits of these proteins is shown in FIG. 22 to illustrate the level of sequence similarity in this family.

These hydrogenase structures have the following general characteristics, which are explained here in terms of hydrogen production, although the reverse reaction, e.g. hydrogen consumption, also occurs. Each hydrogenase consists of a large subunit and a small subunit. The large subunit contains the nickel-iron [NiFe] active site that produces H₂. The small subunit contains three iron-sulfur clusters (two Fe₄S₄ and a Fe₃S₄) that are thought to transfer electrons toward the [NiFe] site by quantum-mechanical tunneling. The most NiFe-distal iron-sulfur cluster is nearest the surface of the protein and is thought to be the initial entry point of electrons; this cluster is coordinated by His185, Cys188, Cys213, and Cys219 in the 2FRV structure.

Based on inspection of the 2FRV structure from D. gigas, it is apparent that the C-terminus of the light chain of the hydrogenase is near the NiFe-distal iron-sulfur cluster. Therefore the C-terminus of the small subunit was chosen as a fusion junction point. A rough docking of the spinach Fe₂S₂ ferredoxin to the D. gigas was performed, in which the distal Fe₄S₄ cluster in the D. gigas enzyme was placed within about 11 Angstroms of the Fe₂S₂ cluster with no steric clashes of the other side chains. This docking indicated that the C-terminus of the hydrogenase small subunit was within less than 40 Angstroms of the N-terminus, but that the line connecting these termini ran through the ferredoxin. An effective linker connecting these termini lies around the ferredoxin during the docking between ferredoxin and the hydrogenase, and the linker should be long enough that numerous conformations of the linker are available in the docked state so that docking is entropically feasible. Therefore in designing a linker to connect the C-terminus of the small subunit to the N-terminus of the ferredoxin, linkers of the form (Gly₄Ser)_{N} were chosen, with N=3, 5, and 7. These linkers have maximal lengths of about 67.5, 112.5, and 157.5 Angstroms, respectively.

Another design consideration was that the Ralstonia MBH has a C-terminal extension that is not found in the Desulfovibrio enzymes. Therefore two versions of the MBH small subunit moiety were designed: one with the extra ‘tail’ and one in which the linker would be placed after the FYDR sequence as indicated in FIG. 22, effectively deleting the “tail”.

The next design element related to the ferredoxin-second linker-PsaE configuration. The proteins PsaC, PsaD, and PsaE are small proteins that sit on top of the larger transmembrane proteins PsaA and PsaB. Two of the three Photosystem I iron-sulfur clusters are within PsaD. Together with PsaA, PsaC, PsaD, and PsaE form a concave surface in which the ferredoxin docks to receive an electron. The geometry of the interaction between the plant-type ferredoxin and Photosystem I is unknown. Therefore, a model was created using the structures 1JB0 for Photosystem 1 and 1A70 for ferredoxin, in which the C-terminus of the ferredoxin was placed as far as possible from the N-terminus of PsaE, while still requiring close contact between the iron-sulfur cluster in ferredoxin and the photocenter-distal iron-sulfur cluster in PsaD. In this docking, the distance between the C-terminus of the ferredoxin and the N-terminus of PsaE was about 45 Angstroms, and the line connecting these termini ran through the ferredoxin. Therefore linkers of the form (Gly₄Ser)_{N} were chosen, with N=3, 5, 7, and 10 were chosen. These linkers have maximal lengths of about 67.5, 112.5, 157.5, and 225 Angstroms, respectively.

As a result of these efforts, several variant fusion proteins were designed. For example, the Ralstonia MBH(truncated)-(Gly₄Ser)⁷-ferredoxin-(Gly₄Ser)₁₀-PsaE protein had the following amino acid sequence:

(SEQ ID NO: 17)
MVETFYEVMRRQGISRRSFLKYCSLTATSLGLGPSFLPQIAHAMETKPRTPVLWLHGLECTCCSESFIR
SAHPLAKDVVLSMISLDYDDTLMAAAGHQAEAILEEIMTKYKGNYILAVEGNPPLNQDGMSCIIGGR
PFIEQLKYVAKDAKAIISWGSCASWGCVQAAKPNPTQATPVHKVITDKPIIKVPGCPPIAEVMTGVITY
MLTFDRIPELDRQGRPKMFYSQRIHDKCYRRPHFDAGQFVEEWDDESARKGFCLYKMGCKGPTTYN
ACSTTRWNEGTSFPIQSGHGCIGCSEDGFWDKGSFYDRGGGGSGGGGSGGGGSGGGGSGGGGSGGG
GSGGGGSAAYKVTLVTPTGNVEFQCPDDVYILDAAEEEGIDLPYSCRAGSCSSCAGKLKTGSLNQDD
QSFLDDDIDEGWVLTCAAYPVSDVTIETHKEEELTAGGGGSGGGGSGGGGSGGGGSGGGGSGGGG
SGGGGSGGGGSGGGGSGGGGSMAIARGDKVRILRPESYWFNEVGTVASVDQSGIKYPVVVRFEKVN
YNGFSGSDGGVNTNNFAEAELQVVAAAAKK

A DNA sequence encoding this protein is constructed by standard techniques; for example by total gene synthesis using a commercial supplier (e.g. DNA 2.0, Blue Heron Biotechnologies, Codon Devices Inc. or TopGene).

A DNA sequence encoding the above protein is used to replace the sequence that encodes the small subunit of the MBH (i.e. the hoxK gene) in the DNA segment encoding the MBH operon described above, within the vector for transformation of Synechococcus. This MBH(HLFLPase) vector is then used to transform a psaE mutant Synechococcus elongatus 7942. The resulting transformants are tested for hydrogen production.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications and patent documents cited herein are incorporated herein by reference as if each such publication or document was specifically and individually indicated to be incorporated herein by reference.

What is claimed is:

1. An isolated bacterial cell comprising a nucleic acid encoding a fusion protein comprising a subunit of photosystem I (PSI) coupled to a heterologous hydrogenase.

2. The bacterial cell of claim 1, wherein said PSI subunit is a PsaE subunit.

3. The bacterial cell of claim 1, wherein said PSI subunit is indirectly coupled to said hydrogenase.

4. The bacterial cell of claim 1, wherein the bacterial cell is a cyanobacterial cell.

5. The bacterial cell of claim 1, wherein the bacterial cell is selected from a Synechococcus elongatus cell, a Synechocystis cell, a Thermosynechococcus elongatus cell, an E. coli cell, a wild cyanobacteria cell, and a Prochloroccus cell.

6. The bacterial cell of claim 1, wherein the bacterial cell is a Synechococcus elongatus PCC7942 cell.

7. The bacterial cell of claim 1, wherein the heterologous hydrogenase is an O₂ tolerant hydrogenase.

8. The bacterial cell of claim 7, wherein the O₂ tolerant hydrogenase is an O₂ tolerant [NiFe] hydrogenase.

9. The bacterial cell of claim 1, wherein the heterologous hydrogenase is an [FeFe] hydrogenase.

10. The bacterial cell of claim 9, wherein said hydrogenase is derived from a Chlamydomonas species, a Clostridium species or a Ralstonia species.

11. The bacterial cell of claim 1, wherein the heterologous hydrogenase is a hoxK subunit of membrane bound hydrogenase (MBH).

12. The bacterial cell of claim 11, wherein the hoxK subunit of MBH is derived from Ralstonia eutropha.

13. The bacterial cell of claim 2, wherein the PsaE subunit is derived from a cyanobacterial PSI.

14. The bacterial cell of claim 1, wherein the PSI subunit is coupled to the heterologous hydrogenase via a linker.

15. The bacterial cell of claim 1, wherein the PSI subunit is linked to the c-terminus of the heterologous hydrogenase.

16. The bacterial cell of claim 14, wherein the heterologous hydrogenase is a hoxK subunit of MBH.

17. The bacterial cell of claim 14, wherein the linker comprises an amino acid sequence.

18. The bacterial cell of claim 1, wherein the nucleic acid is operably linked to a promoter.

19. The bacterial cell of claim 18, wherein the promoter is a photosynthesis-related promoter.

20. The bacterial cell of claim 19, wherein the promoter is psaAB.

21. The bacterial cell of claim 1, wherein the bacterial cell further comprises a nucleic acid encoding a maturation factor.

22. The bacterial cell of claim 1, wherein said hydrogenase comprises one or more mutations relative to the most closely related natural hydrogenase, wherein said mutation confers enhanced enzymatic activity in the presence of oxygen.

23. The bacterial cell of claim 9, wherein the [FeFe] hydrogenase comprises an amino acid alteration relative to the most closely related natural hydrogenase, wherein said alteration places an amino acid with a higher molecular weight than leucine at a position selected from the group 136, 163, 384, 464, and 469 numbered according the sequence of the [FeFe] hydrogenase from Chlamydomonas reinhardtii, wherein said most closely related natural hydrogenase has an amino acid with a molecular weight equal to or less than that of leucine at the corresponding position.

24. The bacterial cell of claim 9, wherein the [FeFe] hydrogenase comprises an amino acid alteration relative to the most closely related natural hydrogenase, wherein said alteration places an amino acid with a higher molecular weight at a position selected from the group 275, 284, 431, 435, 462, 468, and 493 numbered according the sequence of the [FeFe] hydrogenase from Clostridium pasteurianum, wherein said most closely related natural hydrogenase has an amino acid with a molecular weight equal to or less than that of substituted amino acid at the corresponding position.

25. A system for producing biological hydrogen, the system comprising the bacterial cell of claim 1.

26. A method of producing hydrogen, the method comprising:
(a) providing a light source; and
(b) using the isolated bacterial cell of claim 1 to drive the reaction:
6CO₂+12H₂O+photons→C₆H₁₂O₆+6O₂+6H₂O.

27. The method of claim 26, wherein said isolated bacterial cell is a cyanobacterial cell.
